SEQUENCE LISTING (99 sequences)

SEQ ID NO: 1 - DNA, 417 bp, Homo sapiens
gcggcggcggcggcggcctgcggcggcggcggcggcgcggtcggcgggcggcgggcggcggcggcggcgtcggcggcggcggcggctgcgggcggcggcggcggtgcggcgcggccggccgcggcggcggcggcgggcggcggcggcggcggcggcggccggcgggcggcggtcggccgcggcggcggcgcgggcggcggcggcggcggctggcgggcggcggggcggcggcggcggcggcggcggcggcggcggcgcgcgggcgaagcggcggcggcggcggtcggcggcgcggcggcggccggcggcggcggcggcgggcggcggcggcggcggctgcgagcggtggcggcgggcggcggcggcggccggcgcggcggcggcggcggctgcggcggggcggcggggggggcggcg

SEQ ID NO: 2 - DNA, 431 bp, Homo sapiens
ggcggcggcggcggcggcggccggcggcggcggcggcggcggcggcggcgggcggcgggcggcggcggcggcggcggcggcggcggcggcggcgggcggcggcggcgggcggcggcggcgcggcggcggcggcggcggcgggcggcggcggcggcggcggcggcggcgggcggcgggcggccgcggcggcggcgcgggcggcggcggcggcggcggcgggcggcggggcggcggcggcggcggcggcggcggcggcggcggcggcgcggcgcggcggcggcggcggcggcggcgcggcggcggccggcggcggcggcggcgggcggcggcggcggcggctgcggcggcggcggcgggcggcggcggcggccggcggcggcggcggcggcggcggcggcggggcggcggggggggcggcgcgggcggcggcg

SEQ ID NO: 3 - DNA, 426 bp, Homo sapiens
ggcggcgggcggcggccggcggcggcggcggcggcggcggcggcggcggcggcggccggcggcactggcggcggcggcggcggcggcggcgcggcgtgcggcgtcggcggcggcggcgggcggcggcggcggcggcggcggcggcggtcggcggcggcggcgggcggcggcgcgcggcggcgcggcggcggcggcggcggcggcgggctggcgcggcggcgcggatgcggcggcggcggcggcggcggcgggcggcggcggcggcggcggcggggcggcggcgcggcgcggcgggcggcggcggcggcggcgcggctgcggcggcggcggctgcggcggctgcggcggcggcggcggcgtctgcggcggcggcggcggcggcggtggcggcggcgcggcggctgcggcggcgcggcggcggcggcg

SEQ ID NO: 4 - DNA, 433 bp, Homo sapiens
gcggcggcggcgcggcggccggcggcggcggcggcggcggcggcggcggcggcggcggccggcggcggcgcggcggcggcggcggcggcggcggcgcggcggcggcggcggcggcggcggcgggcggcggcggcggcggcggcggcggcggcggcggcggcggcgggcggcggcgcgcggcggcgcggcggcggcggcggcggcggcgggcggcggcggcggcggcggcggcggcggcggcggcggcggcggcgggcggcggcggcggcggcggcggggcggcggcggcggcggcggcgggcggcggcggcggcggcgcggcggcggcggcggcggcggcggcggcggcggcggcggcggcggcggcggcggcggcggcggcggcggcggcggcggcggcgcggcggcggcggcggcggcggcggcggcggcg

SEQ ID NO: 5 - DNA, 428 bp, Homo sapiens
cggcggcggcggctgtgcggaggcggcgggcggcggcggggcggcggcgcggcggcggcggcggcggcggcgtcggcggcggcggcggcggcgcgccggcggcggcgcggcggcggcgggcgggcggcggcggcggcggcggcggcggcggcggcggcggcgggcggcggcggcggcggcggcgcggcggcggcgcggcggcggcaggcggcggcggcggaggcggctttggcttcggcggcatggcggcggcggcggcggcggatggcggcggcggcggcggcggcggcggcggcggcggcggcgcggcggaggcggcggcggggcgcggcggcggcggctgccggcggcggcgggcggcggggcggcggcggtccggccggcggcggcagagcggcggcaggcggcggccggcggcggcggcggcg

SEQ ID NO: 6 - DNA, 436 bp, Homo sapiens
ggcggcggcggcggtgcggcggcggcgggcggcggcggcggcggcggcggcggcggcggcggcggcggcggcggcggcggcggcggcggcggcgcgccggcggcggcggcggcggcggcgggcgggcggcggcggcggcggcggcggcggcggcggcggcggcgggcggcggcggcggcggcggcgcggcggcggcggcggcggcggcggcggcggcggcggcggcggcggcggcggcggcggcgtggcggcggcggcggcggcggcgggcggcggcggcggcggcggcggcggcggcggcggcggcggcggcggaggcggcggcggggcggcggcggcggcggctgccggcggcggcgggcggcggcggcggcggcggcgcggcggcggcggcggcggcggcggcggcgggcggcggccggcggcggcggcggcg

SEQ ID NO: 7 - DNA, 460 bp, Homo sapiens
ggcggcggcggcggcggcggcggcggcgcggcgcgcggcggcggcggcggcggcggcggctgcggtcgcggcggcggcgcggcgcggcggcgtcggccggcggcggcggcggcggcgggcggcggcggcggcgggcggcggcggcggcgggcggcggcggcggctgcgcggcggcggcggcggcggcgcggcgcggcgcggcggcggcggcgggcggcggcggcggccggcggcggcggcggcggggcggcggcggcgcgggggggcggcggcggcggcggcggcggcggcggcgcggcggcggcggcggcggcggcgggcggcggcggcggcgcggcggcgggccggcggcggcggcggcggcggcggcgcggcggcggcggcggcggcggccggcggcggcggcggcggcggcggcggcggcggggggggcgggcggggaggcgcggggcggcggcgg

SEQ ID NO: 8 - DNA, 455 bp, Homo sapiens
ggcggcggcggcggcggcggcggcggcgcggcggcggcggcggcggcggcggcggcggcggcggcgcggcggcggcgcggcgcggcggcggcggccggcggcggcggcggcggcgggcggcggcggcggcgggcggcggcggcggcgggcggcggcggcggcggcgcggcggcggcggcggcggcgcggcgcggcgcggcggcggcggcggcggcggcggcggccggcggcggcggcggcggggcggcggcggcggcggcggcggcggcggcggcggcggcggcggcggcgcggcggcggcggcggcggcggcgggcggcggcggcggcgcggcggcgggcggcggcggcggcggcggcggcggcgcggcggcggcggcggcggcggccggcggcggcggcggcggcggcggcggcggcggggggggcgggcggcggaggcgcggggcggcggcg

SEQ ID NO: 9 - DNA, 479 bp, Homo sapiens
cggcggcgcggcggcggcggcggcgcggcggcggcggcggcggcggcggcggccgtgcggcggcggctgcggcggcggcggcggcggcggcggggaccggcggcggcggcgggcggcggcggcggcggcggcggggcggcggcggcggcggcggcgggcggcggcggcggcggcggcgcggccggcggcgggcgcggcggcggcggcggctttggcggcggcggcggggaggcggcggcggcggcggcggcggcggcggcggcggcggcgtgcggcgggcggcggcggggcggcgggcggcggctggcggcggcggggcggcggcggcggccggcggagcggcccggcgcgcggcggcggcggcggcggcggcggcgggcggcggcggcggcggcggcggcggcaggcggcggcggcggcggcggcggcggccggcggggcggcgaggcggcggcgcggcggcgtggcggccggcggcg

SEQ ID NO: 10 - DNA, 461 bp, Homo sapiens
ggcggcggcggcggcggcggcggcggcgcggcggcggcggcggcggcggcggcggccggcggcggcggcggcggcggcggcggcggcggcggcggggcggcggcggcggcgggcggcggcggcggcggcggcgggcggcggcggcggcggcggcggcggcggcggcggcggcggcgcggcggcggcgggcgcggcggcggcggcggcggcggcggcggcggcgaggcggcggcggcggcggcggcggcggcggcggcggcggcggcggcggcggcggcggggcggcggcggcggcggcggcggcggcggcggcggcggcggcggcggagcggcggcggcggcggcggcggcggcggcggcggcgggcggcggcggcggcggcggcggcggcggcggcggcggcggcggcggcggcggcggcggggcggcgggcggcggcgcggcggcgggcggcggcggcg

SEQ ID NO: 11 - DNA, 50 bp, Homo sapiens
gtggtgactcctctatcgggacgcccctcccattgtatctggcccaggct

SEQ ID NO: 12 - DNA, 50 bp, Homo sapiens
gggagagtggggctcctctatcgggaccccctccccattggatctgccca

SEQ ID NO: 13 - DNA, 50 bp, Homo sapiens
gggctcctctatcggacccccttcgcccattcgtggatctgcccatcgcg

SEQ ID NO: 14 - DNA, 50 bp, Homo sapiens
agagtggggctcctctatcgggaccccctccccatgtggatctgcccatc

SEQ ID NO: 15 - DNA, 50 bp, Homo sapiens
agagagagtggggatactctaatcgggaccccctccccatgggatctgcc

SEQ ID NO: 16 - DNA, 50 bp, Homo sapiens
agagagtggggctcctctatcgggaccccctccccatgtggatctgccca

SEQ ID NO: 17 - DNA, 50 bp, Homo sapiens
gagagggggctcctctatcgtgaccccctccccatgttgtctgcccccca

SEQ ID NO: 18 - DNA, 50 bp, Homo sapiens; identical to SEQ ID NO: 16

SEQ ID NO: 19 - DNA, 50 bp, Homo sapiens
agagtggggctcctatatcgggaccccctccccatgtgatctgcccaggt

SEQ ID NO: 20 - DNA, 50 bp, Homo sapiens; identical to SEQ ID NO: 16

SEQ ID NO: 21 - DNA, 50 bp, Homo sapiens
cgggcgcggcgaaccgagaatatgcccgccctgcgcagctctgactgctg

SEQ ID NO: 22 - DNA, 50 bp, Homo sapiens
aaccgagaagatgcccgccctgcgccgctctctgctgtgggcgctgctgg

SEQ ID NO: 23 - DNA, 50 bp, Homo sapiens
accggagatggccccgccctgcgccgtgctctgctgtgggggctgctggc

SEQ ID NO: 24 - DNA, 50 bp, Homo sapiens
accgagaagatgcccgccctgcgccgctctgctgtgggcgctgctggcgc

SEQ ID NO: 25 - DNA, 50 bp, Homo sapiens
accgagaagatgccacgccatgcgccgctctgatgtgggcgatgctggcg

SEQ ID NO: 26 - DNA, 50 bp, Homo sapiens; identical to SEQ ID NO: 24

SEQ ID NO: 27 - DNA, 50 bp, Homo sapiens
accgagaagaatgcccgccctggcccgctctgctgtgggcgctgcttctg

SEQ ID NO: 28 - DNA, 50 bp, Homo sapiens; identical to SEQ ID NO: 24

SEQ ID NO: 29 - DNA, 50 bp, Homo sapiens
accgagaagatttgcccgccctgcgacgctctgatgtgggctgggctggc

SEQ ID NO: 30 - DNA, 50 bp, Homo sapiens
accgagaagatgcccgccctgcgccgctctgctgtgggctgctgctggcg

SEQ ID NO: 31 - DNA, 20 bp, Artificial Sequence
  Primer
agcgcccacagcagagcggc

SEQ ID NO: 32 - DNA, 38 bp, Artificial Sequence
  Primer
ccgggagctgcatgtgtcagaggcggcggcggcggcgg

SEQ ID NO: 33 - DNA, 23 bp, Artificial Sequence
  Primer
ccgggagctgcatgtgtcagagg

SEQ ID NO: 34 - DNA, 23 bp, Artificial Sequence
  Primer
cgctagaaggagtgtggtccacc

SEQ ID NO: 35 - DNA, 38 bp, Artificial Sequence; identical to SEQ ID NO: 32
  Primer

SEQ ID NO: 36 - DNA, 23 bp, Artificial Sequence
  Primer
ccgggagctgcatgtgtcagagg

SEQ ID NO: 37 - DNA, 27 bp, Artificial Sequence
  Primer
ggagggaggagaagctggaggtagacg

SEQ ID NO: 38 - DNA, 38 bp, Artificial Sequence; identical to SEQ ID NO: 32
  Primer

SEQ ID NO: 39 - DNA, 23 bp, Artificial Sequence
  Primer
ccgggagctgcatgtgtcagagg

SEQ ID NO: 40 - DNA, 28 bp, Artificial Sequence
  Primer
tcaggcgctcagctccgtttcggtttca

SEQ ID NO: 41 - DNA, 38 bp, Artificial Sequence
  Primer
ccgggagctgcatgtgtcagaggccgccgccgccgccg

SEQ ID NO: 42 - DNA, 23 bp, Artificial Sequence
  Primer
ccgggagctgcatgtgtcagagg

SEQ ID NO: 43 - DNA, 20 bp, Artificial Sequence
  Primer
gtgtgctgctcgcgtctttg

SEQ ID NO: 44 - DNA, 20 bp, Artificial Sequence
  Primer
ctacaattctctaaagcagg

SEQ ID NO: 45 - DNA, 20 bp, Artificial Sequence
  Primer
gtgtgctgctcgcgtctttg

SEQ ID NO: 46 - DNA, 20 bp, Artificial Sequence
  Primer
gtgtgggtgggatggggaag

SEQ ID NO: 47 - DNA, 20 bp, Artificial Sequence
  Primer
tattaaacggatgacactcc

SEQ ID NO: 48 - DNA, 20 bp, Artificial Sequence
  Primer
ctggtccacttctgaaattc

SEQ ID NO: 49 - DNA, 20 bp, Artificial Sequence
  Primer
gaatttcagaagtggaccag

SEQ ID NO: 50 - DNA, 20 bp, Artificial Sequence
  Primer
ctacaattctctaaagcagg

SEQ ID NO: 51 - DNA, 23 bp, Artificial Sequence
  Primer
ttggagtgtgcagagggataagg

SEQ ID NO: 52 - DNA, 19 bp, Artificial Sequence
  Primer
cgcaggccagcttctctcg

SEQ ID NO: 53 - DNA, 23 bp, Artificial Sequence
  Primer
ttggagtgtgcagagggataagg

SEQ ID NO: 54 - DNA, 21 bp, Artificial Sequence
  Primer
tggattccacccccgcggctc

SEQ ID NO: 55 - DNA, 23 bp, Artificial Sequence
  Primer
ggagtcaggacagatgtgtacac

SEQ ID NO: 56 - DNA, 21 bp, Artificial Sequence
  Primer
gtggttatggcctgtcgctgg

SEQ ID NO: 57 - DNA, 23 bp, Artificial Sequence
  Primer
ggagtcaggacagatgtgtacac

SEQ ID NO: 58 - DNA, 25 bp, Artificial Sequence
  Primer
gatgcttgactgtgagaaagcagag

SEQ ID NO: 59 - DNA, 25 bp, Artificial Sequence
  Primer
ctctgctttctcacagtcaagcatc

SEQ ID NO: 60 - DNA, 21 bp, Artificial Sequence
  Primer
gtggttatggcctgtcgctgg

SEQ ID NO: 61 - DNA, 20 bp, Artificial Sequence
  Primer
tactcaccatgcgcgggggt

SEQ ID NO: 62 - DNA, 38 bp, Artificial Sequence
  Primer
ccgggagctgcatgtgtcagagggcctgtgcttcggac

SEQ ID NO: 63 - DNA, 23 bp, Artificial Sequence
  Primer
ccgggagctgcatgtgtcagagg

SEQ ID NO: 64 - DNA, 25 bp, Artificial Sequence
  Primer
cgcagcccgagtttcccacctttta

SEQ ID NO: 65 - DNA, 48 bp, Artificial Sequence
  Primer
ccgggagctgcatgtgtcagaggctcgctagaaggagtgtggtccacc

SEQ ID NO: 66 - DNA, 23 bp, Artificial Sequence
  Primer
ccgggagctgcatgtgtcagagg

SEQ ID NO: 67 - DNA, 22 bp, Artificial Sequence
  Primer
gccaccctctcgtctcgcgctg

SEQ ID NO: 68 - DNA, 43 bp, Artificial Sequence
  Primer
ccgggagctgcatgtgtcagaggcgaggaaaagcaagagcaac

SEQ ID NO: 69 - DNA, 23 bp, Artificial Sequence
  Primer
ccgggagctgcatgtgtcagagg

SEQ ID NO: 70 - DNA, 19 bp, Artificial Sequence
  Primer
ttgcgcctgtgcttcggac

SEQ ID NO: 71 - DNA, 20 bp, Artificial Sequence
  Primer
tactcaccatgcgcgggggt

SEQ ID NO: 72 - DNA, 16 bp, Artificial Sequence
  Barcode
atgctgatgacgcgct

SEQ ID NO: 73 - DNA, 16 bp, Artificial Sequence
  Barcode
gacagcatctgcgctc

SEQ ID NO: 74 - DNA, 16 bp, Artificial Sequence
  Barcode
agcgtctgacgtgagt

SEQ ID NO: 75 - DNA, 16 bp, Artificial Sequence
  Barcode
tcgatatacgacgtgc

SEQ ID NO: 76 - DNA, 16 bp, Artificial Sequence
  Barcode
tcgtcatacgctctag

SEQ ID NO: 77 - DNA, 16 bp, Artificial Sequence
  Barcode
cgactacgtacagtag

SEQ ID NO: 78 - DNA, 16 bp, Artificial Sequence
  Barcode
gcgtagacagactaca

SEQ ID NO: 79 - DNA, 16 bp, Artificial Sequence
  Barcode
acagtatgatgtactc

SEQ ID NO: 80 - DNA, 16 bp, Artificial Sequence
  Barcode
gtctgatagatacaga

SEQ ID NO: 81 - DNA, 16 bp, Artificial Sequence
  Barcode
ctgcgcagtacgtgca

SEQ ID NO: 82 - DNA, 16 bp, Artificial Sequence
  Barcode
gtacatatgcgtctgt

SEQ ID NO: 83 - DNA, 16 bp, Artificial Sequence
  Barcode
gagactagagatagtg

SEQ ID NO: 84 - DNA, 16 bp, Artificial Sequence
  Barcode
tacgcgtgtacgcaga

SEQ ID NO: 85 - DNA, 16 bp, Artificial Sequence
  Barcode
tgtcactcatctgagt

SEQ ID NO: 86 - DNA, 16 bp, Artificial Sequence
  Barcode
gcacatacacgctcac

SEQ ID NO: 87 - DNA, 16 bp, Artificial Sequence
  Barcode
gctcgtcgcgcgcaca

SEQ ID NO: 88 - DNA, 16 bp, Artificial Sequence
  Barcode
acagtgcgctgtctat

SEQ ID NO: 89 - DNA, 16 bp, Artificial Sequence
  Barcode
tcacactctagagcga

SEQ ID NO: 90 - DNA, 16 bp, Artificial Sequence
  Barcode
tcacatatgtatacat

SEQ ID NO: 91 - DNA, 16 bp, Artificial Sequence
  Barcode
cgctgcgagagacagt

SEQ ID NO: 92 - DNA, 16 bp, Artificial Sequence
  Barcode
cgctgcgagagacagt

SEQ ID NO: 93 - DNA, 16 bp, Artificial Sequence
  Barcode
gcagactctcacacgc

SEQ ID NO: 94 - DNA, 22 bp, Artificial Sequence
  Primer
accgagaagatgcccgccctgc

SEQ ID NO: 95 - DNA, 22 bp, Artificial Sequence
  Primer
cgcgcctcggaaagaataacag

SEQ ID NO: 96 - DNA, 22 bp, Artificial Sequence
  Primer
accgagaagatgcccgccctgc

SEQ ID NO: 97 - DNA, 21 bp, Artificial Sequence
  Primer
aactgcccacctccctgcacc

SEQ ID NO: 98 - DNA, 21 bp, Artificial Sequence
  Primer
cggcagcaagtctcagaaact

SEQ ID NO: 99 - DNA, 22 bp, Artificial Sequence
  Primer
cgcgcctcggaaagaataacag

(19) United States
(12) Patent Application Publication
(10) Pub. No.: US 2022/0307085 A1
(43) Pub. Date: Sep. 29, 2022

(54) Method and Kit for Determining Neuromuscular Disease in Subject

(71) Applicant: The University of Tokyo, Tokyo (JP)

(72) Inventors: Shoji Tsuji, Tokyo (JP); Hiroyuki Ishiura, Tokyo (JP)

(21) Appl. No.: 17/607,979

(22) PCT Filed: May 1, 2020
(86) PCT No.: PCT/JP2020/018412

§ 371 Date: Nov. 1, 2021

Related U.S. Application Data
(60) Provisional application No. 62/842,110, filed on May 2, 2019.

(51) Int. Cl. C12Q 1/6883

(52) U.S. Cl. CPC C12Q 1/6883; C12Q 2600/156

(57) ABSTRACT

A method for determining a neuromuscular disease accompanied with a repeat expansion of CGG in a nucleic acid in a subject comprising detecting a repeat expansion of CGG or a complementary sequence thereof in a nucleic acid sample from the subject.

### TECHNICAL FIELD

A method and a kit for determining a neuromuscular disease in a subject are disclosed.

### BACKGROUND ART

Noncoding repeat expansions cause various neuromuscular diseases including myotonic dystrophies, fragile X tremor/ataxia syndrome (FXTAS), some spinocerebellar ataxias, amyotrophic lateral sclerosis, and benign adult familial myoclonic epilepsies (BAFME).

### CITATION LIST

### Non Patent Literature

[NPL 1]
Loureiro, J. R., Oliveira, C. L. & Silveira, I., “Unstable repeat expansions in neurodegenerative diseases: nucleocytoplasmic transport emerges on the scene,” Neurobiol. Aging 39, 174-183 (2016).
[NPL 2]
Vissers, L. E., et al., “A de novo paradigm for mental retardation,” Nat. Genet. 42, 1109-1112 (2010).
[NPL 3]
Lindenberg, R., Rubinstein, L. J., Herman, M. M. & Haydon, G. B., “A light and electron microscopy study of an unusual widespread nuclear inclusion body disease. A possible residuum of an old herpesvirus infection,” Acta Neuropathol. 10; 54-73 (1968).
[NPL 4]
Haltia, M., Somer, H., Palo, J. & Johnson, W. G., “Neuronal intranuclear inclusion disease in identical twins,” Ann. Neurol. 15; 316-321 (1984).
[NPL 5]
Sone, J. et al., “Clinicopathological features of adult-onset neuronal intranuclear inclusion disease,” Brain 139, 3170-3186 (2016).
[NPL 6]
Takahashi-Fujigasaki, J., Nakano, Y., Uchino, A. & Murayama, S., “Adult-onset neuronal intranuclear hyaline inclusion disease is not rare in older adults,” Geriatr. Gerontol. Int. 16 Suppl 1, 51-56 (2016).
[NPL 7]
Kimber, T. E. et al., «Familial neuronal intranuclear inclusion disease with ubiquitin positive inclusions,” J. Neurol. Sci. 160, 33-40 (1998).
[NPL 8]
Sone, J. et al., “Neuronal intranuclear hyaline inclusion disease showing motor-sensory and autonomic neuropathy,” Neurology 65, 1538-1543 (2005).
[NPL 9]
Yamaguchi, N. et al., “An autopsy case of familial neuronal intranuclear inclusion disease with dementia and neuropathy,” Intern. Med. in press (doi: 10.2169/internalmedicine.1141-18).
[NPL 10]
Sone, J. et al., “Neuronal intranuclear inclusion disease cases with leukoencephalopathy diagnosed via skin biopsy,” J. Neurol. Neurosurg. Psychiatry 85, 354-356 (2014).
[NPL 11]
Sone, J. et al., “Skin biopsy is useful for the antemortem diagnosis of neuronal intranuclear inclusion disease,” Neurology 76, 1372-1376 (2011).
[NPL 12]
Nakano, Y. et al., “PML nuclear bodies are altered in adult-onset neuronal intranuclear hyaline inclusion disease,” J. Neuropathol. Exp. Neurol. 76, 585-594 (2017).
[NPL 13]
Takumida, H. et al., “Case of a 78-year-old woman with a neuronal intranuclear inclusion disease,” Geriatr. Gerontol. Int. 17, 2623-2625 (2017).
[NPL 14]
Sugiyama, A. et al., “MR imaging features of the cerebellum in adult-onset neuronal intranuclear inclusion disease: 8 cases,” Am. J. Neuroradiol. 38, 2100-2104 (2017).
[NPL 15]
Hunsaker, M. R. et al., “Widespread non-central nervous system organ pathology in fragile X premutation carriers with fragile X-associated tremor/ataxia syndrome and CGG knock-in mice,” Acta Neuropathol. 122, 467-479 (2011).
[NPL 16]
Hagerman, R. J. et al., “Intention tremor, parkinsonism, and generalized brain atrophy in male carriers of fragile X,” Neurology 57, 299-301 (2001).
[NPL 17]
Doi, K. et al., “Rapid detection of expanded short tandem repeats in personal genomics using hybrid sequencing,” Bioinformatics 30, 815-822 (2014).
[NPL 18]
Ishiura, H. et al., “Expansions of intronic TTTCA and TTTTA repeats in benign adult familial myoclonic epilepsy,” Nat. Genet. 50, 581-590 (2018).
[NPL 19]
Vandepoele, K., Van Roy, N., Staes, K., Speleman, F. & van Roy, F., “A novel gene family NBPF: intricate structure generated by gene duplication during primate evolution,” Mol. Biol. Evol. 22; 2265-75 (2005).
[NPL 20]
Fiddes, I. T. et al., “Human-specific NOTCH2NL genes affect Notch signaling and cortical neurogenesis,” Cell 173, 1356-1369 (2018).
[NPL 21]
Suzuki, I. K. et al., “Human-specific NOTCH2NL genes expand cortical neurogenesis through Delta/Notch regulation,” Cell 173, 1370-1384 (2018).
[NPL 22]
Li, H., “Minimap2: pairwise alignment for nucleotide sequences,” Bioinformatics in press (doi: 10.1093/bioinformatics/bty191).
[NPL 23]
Koren, S. et al., “Canu: scalable and accurate long-read assembly via adaptive k-mer weighting and repeat separation,” Genome Res. 27, 722-736 (2017).
[NPL 24]
Flusberg, B. A., et al., “Direct detection of DNA methylation during single-molecule, real-time sequencing,” Nat. Methods 7, 461-465 (2010).
[NPL 25]
Suzuki, Y, et al., “Agln: measuring the landscape of CpG methylation of individual repetitive elements,” Bioinformatics 32, 2911-2919 (2016).
[NPL 26]
Schuffler, M. D., Bird, T. D., Sumi, S. M. & Cook, A., “A familial neuronal disease presenting as intestinal pseudoobstruction,” Gastroenterology 75, 889-898 (1978).
[NPL 27]
Satoyoshi, E. & Kinoshita, M., “Oculopharyngodistal myopathy,” Arch. Neurol. 34, 89-92 (1977).
[NPL 28]
Durmus, H. et al., “Oculopharyngodistal myopathy is a distinct entity: clinical and genetic features of 47 patients,” Neurology 76, 227-235 (2011).
[NPL 29]
Zhao, J. et al., “Clinical and muscle imaging findings in 14 mainland Chinese patients with oculopharyngodistal myopathy,” PLoS One 10, e0128629 (2015).
[NPL 30]
Satoyoshi, E., “Distal myopathy,” Tohoku J. Exp. Med. 161 Suppl, 1-19 (1990).
[NPL 31]
Brais, B. et al., “Short GCG expansions in the PABP2 gene cause oculopharyngeal muscular dystrophy,” Nat. Genet. 18, 164-167 (1998).
[NPL 32]
Seltzer, M. M., et al., “Prevalence of CGG expansions of the FMR1 gene in a US population-based sample,” Am. J. Med. Genet. B Neuropsychiatr. Genet. 159B, 589-597 (2012).
[NPL 33]
Beck, J. et al., “Large C9orf72 hexanucleotide repeat expansions are seen in multiple neurodegenerative syndromes and are more frequent than expected in the UK population,” Am. J. Hum. Genet. 92, 345-353 (2013).
[NPL 34]
Renton, A. E. et al., “A hexanucleotide repeat expansion in C9ORF72 is the cause of chromosome 9p21-linked ALS-FTD,” Neuron 72, 257-268.
[NPL 35]
Jacquemont, S. et al., “Penetrance of the fragile X-associated tremor/ataxia syndrome in a premutation carrier population,” JAMA 291, 460-469 (2004).
[NPL 36]
Coffey, S. M. et al., “Expanded clinical phenotype of women with the FMR1 premutation,” Am. J. Med. Genet. A 146A; 1009-1016 (2008).
[NPL 37]
DeJesus-Hernandez, M. et al., “Expanded GGGGCC hexanucleotide repeat in noncoding region of C9ORF72 causes chromosome 9p-linked FTD and ALS,” Neuron 72, 245-256 (2011).
[NPL 38]
Fratta, P. et al., “Screening a UK amyotrophic lateral sclerosis cohort provides evidence of multiple origins of the C9orf72 expansion,” Neurobiol. Aging 36, el-7 (2015).
[NPL 39]
Buxton, J. et al., “Detection of an unstable fragment of DNA specific to individuals with myotonic dystrophy,” Nature 355, 547-548 (1992).
[NPL 40]
Zu, T. et al., “Non-ATG-initiated translation directed by microsatellite expansions,” Proc. Natl. Acad. Sci. U.S.A 108, 260-265 (2011).
[NPL 41]
Todd, P. K. et al., “CGG repeat-associated translation mediates neurodegeneration in fragile X tremor ataxia syndrome,” Neuron 78; 440-455 (2013).
[NPL 42]
Uyama, E., Uchino, M., Chateau, D., & Tome, F. M., “Autosomal recessive oculopharyngodistal myopathy in light of distal myopathy with rimmed vacuoles and oculopharyngeal muscular dystrophy,” Neuromuscul. Disord. 8, 119-125 (1998).
[NPL 43]
Jin, P. et al., “Pur alpha binds to rCGG repeats and modulates repeat-mediated neurodegeneration in a Drosophila model of fragile X tremor/ataxia syndrome,” Neuron 55, 556-564 (2007).
[NPL 44]
Sofola, O. A. et al., “RNA-binding proteins hnRNP A2/B1 and CUGBP1 suppress fragile X CGG premutation repeat-induced neurodegeneration in a Drosophila model of FXTAS,” Neuron 55, 565-571 (2007).
[NPL 45]
Bahlo, M. et al., “Recent advances in the detection of repeat expansions with short-read next-generation sequencing,” F1000Res. 7 (F1000 Faculty Rev), 736 (2018).
[NPL 46]
Mitsuhashi, S. et al., “Tandem-genotypes: robust detection of tandem repeat expansions from long DNA reads,” Genome Biol. 20, 58 (2019).
[NPL 47]
Sznajder, L. J. et al., “Intron retension induced by microsatellite expansions as a disease biomarker,” Proc. Natl. Acad. Sci. U.S.A 115, 4234-4239 (2018).
[NPL 48]
Fukuda, Y. et al., “SNP HiTLink: a high-throughput linkage analysis system employing dense SNP data,” BMC Bioinformatics 10, 121 (2009).
[NPL 49]
Gudbjartsson, D. F., Thorvaldsson, T., Kong, A., Gunnarsson, G. & Ingolfsdottir, A. Allegro version 2, Nat. Genet. 37, 1015-1016 (2005).
[NPL 50]
Kent, W. J., “BLAT—the blast-like alignment tool,” Genome Res. 14, 656-664 (2002).
[NPL 51]
Larkin, M. A., et al., “Clustal W and Clustal X version 2.0,” Bioinformatics 23, 2947-2948 (2007).
[NPL 52]
Vaser, R., Sović, I., Nagarajan, N., and Šikić, M., “Fast and accurate de novo genome assembly from long uncorrected reads,” Genome Res. 27, 737-746 (2017).
[NPL 53]
Benson, G., “Tandem repeat finder: a program to analyze DNA sequences,” Nucleic Acids Res. 27, 573-580 (1999).
[NPL 54]
Frey, U. H., Bachmann, H. S., Peters, J., & Siffert, W, “PCR-amplification of GC-rich regions: ‘slowdown PCR’,” Nat. Protoc. 3; 1312-1317 (2008).
[NPL 55]
Su, J., et al., “CpG_MP2: identification of CpG methylation patterns of genomic regions from high-throughput bisulfite sequencing data,” Nucleic Acids Res. 41, e4 (2013).
[NPL 56]
Dobin, A. et al., “STAR: ultrafast universal RNA-seq aligner,” Bioinformatics 29, 15-21 (2013).
[NPL 57]
Li, H., et al., “The Sequence Alignment/Map format and SAMtools,” Bioinformatics 25, 2078-2079 (2009).
[NPL 58]
Robinson, J. T. et al., “Integrative Genomic Viewer,” Nat. Biotechnol. 29, 24-26 (2011).
[NPL 59]
Miyazawa, H., et al., “Homozygosity haplotype allows a genomewide search for the autosomal segments shared among patients,” Am. J. Hum. Genet. 80, 1090-1102 (2007).
[NPL 60]
Satoyoshi, E. & Kinoshita, M., “Oculopharyngodistal myopathy,” Arch. Neurol.34, 89-92 (1977).
[NPL 61]
Amato, A. A., Jackson, C. E., Ridings, L. W. & Barohn, R. J., “Childhood-onset oculopharyngodistal myopathy with chronic intestinal pseudo-obstruction,” Muscle Nerve 18, 842-847 (1995).
[NPL 62]
Thevathasan, W, et al., “Oculopharyngodistal myopathy—a possible association with cardiomyopathy,” Neuromuscul. Disord.21, 121-125 (2011).

### SUMMARY OF INVENTION

### Technical Problem

The aim of the present invention is to provide a new method for determining a neuromuscular disease in a subject are disclosed.

### Solution to Problem

Inspired by the striking similarities in the clinical and neuroimaging findings between neuronal intranuclear inclusion disease (NIID) and FXTAS caused by noncoding CGG repeat expansions in FMR1, the present inventors directly searched for repeat expansion mutations, and identified noncoding CGG repeat expansions in NBPF19 (NOTCH2NLC) as the causative mutations for NIID. Further prompted by the similarities in the clinical and neuroimaging findings with NIID, the present inventors identified similar noncoding CGG repeat expansions in two other diseases, oculopharyngeal myopathy with leukoencephalopa (OPML) and oculopharyngodistal myopathy (OPDM) in LOC642361/NUTM2B-AS1 and LRP12, respectively. These findings expand the present inventor's knowledge on the clinical spectra of diseases caused by expansions of the same repeat motif and further highlight the role of direct search for expanded repeats in identifying genes underlying diseases.

An aspect of the present disclosure relates to a method for determining, diagnosing, or aiding to diagnose a neuromuscular disease accompanied with a repeat expansion of CGG in a nucleic acid in a subject comprising detecting a repeat expansion of CGG or a complementary sequence thereof in a nucleic acid sample from the subject. The neuromuscular disease may be selected from the group consisting of neuronal intranuclear inclusion disease, oculopharyngodistal myopathy, and oculopharyngeal myopathy with leukoencephalopathy.

An aspect of the present disclosure relates to a method for treating a neuromuscular disease accompanied with a repeat expansion of CGG in a nucleic acid in a subject comprising detecting a repeat expansion of CGG or a complementary sequence thereof in a nucleic acid sample from the subject, and if the repeat expansion is detected, administering a pharmaceutical composition for treating the neuromuscular disease to the subject. The neuromuscular disease may be selected from the group consisting of neuronal intranuclear inclusion disease, oculopharyngodistal myopathy, and oculopharyngeal myopathy with leukoencephalopathy.

In the above method, the nucleic acid sample may be a chromosome DNA. In the above method, the repeat expansion of CGG may be in an intron of a gene from the subject.

In the above method, the neuromuscular disease may be neuronal intranuclear inclusion disease and the repeat expansion of CGG may be in 5′ untranslated region of NBPF19 gene. In the above method, the neuromuscular disease may be neuronal intranuclear inclusion disease and the repeat expansion may be greater than 70 repeats.

In the above method, the neuromuscular disease may be oculopharyngodistal myopathy and the repeat expansion of CGG may be in 5′ untranslated region of LRP12 gene. In the above method, the neuromuscular disease may be oculopharyngodistal myopathy and the repeat expansion is greater than 70 repeats.

In the above method, the neuromuscular disease may be oculopharyngeal myopathy with leukoencephalopathy and the repeat expansion of CGG may be in 5′ untranslated region of LOC642361 gene and/or NUTM2B-AS1 gene. In the above method, the neuromuscular disease may be oculopharyngeal myopathy with leukoencephalopathy and the repeat expansion may be greater than 70 repeats.

An aspect of the present disclosure relates to a kit for determining or diagnosing a neuromuscular disease accompanied with a repeat expansion of CGG in a nucleic acid in a subject comprising a nucleic acid reagent configured to detect a repeat expansion of CGG or a complementary sequence thereof in a nucleic acid sample from the subject. The neuromuscular disease may be selected from the group consisting of neuronal intranuclear inclusion disease, oculopharyngodistal myopathy, and oculopharyngeal myopathy with leukoencephalopathy.

In the above kit, the nucleic acid sample may be a chromosome DNA. In the above kit, the nucleic acid reagent may comprise a PCR primer configured to detect the repeat expansion of CGG or the complementary sequence thereof. In the above kit, the PCR primer may comprise a complementary sequence of CGG or a complementary sequence thereof. In the above kit, the nucleic acid reagent may comprise a probe configured to target a sequence flanking the repeat expansion of CGG or a complementary sequence thereof. In the above kit, the repeat expansion of CGG may be in an intron of a gene from the subject.

In the above kit, the neuromuscular disease may be neuronal intranuclear inclusion disease and the repeat expansion of CGG may be in 5′ untranslated region of NBPF19 gene. In the above kit, the neuromuscular disease may be neuronal intranuclear inclusion disease and the repeat expansion may be greater than 70 repeats.

In the above kit, the neuromuscular disease may be oculopharyngodistal myopathy and the repeat expansion of CGG may be in 5′ untranslated region of LRP12 gene. In the above kit, the neuromuscular disease may be oculopharyngodistal myopathy and the repeat expansion may be greater than 70 repeats.

In the above kit, the neuromuscular disease may be oculopharyngeal myopathy with leukoencephalopathy and the repeat expansion of CGG may be in 5′ untranslated region of LOC642361 gene and/or NUTM2B-AS1 gene. In the above kit, the neuromuscular disease may be oculopharyngeal myopathy with leukoencephalopathy and the repeat expansion is greater than 70 repeats.

### BRIEF DESCRIPTION OF DRAWINGS

FIG. 1 shows a brain MRI of patients with FXTAS, NIID, OPML, and OPDM. Representative brain T2-weighted images (T2WI) and diffusion-weighted images (DWI) of patients with FXTAS [fragile X tremor/ataxia syndrome, a 64-year-old male with mild expansion (premutation) of CGG repeats in FMR1], NIID (neuronal intranuclear inclusion disease, a 72-year-old female with expanded CGG repeats in NBPF19), OPML (oculopharyngeal myopathy with leukoencephalopathy, a 60-year-old female with CGG/CCG repeat expansion in LOC642361/NUTM2B-AS1), and OPDM (oculopharyngodistal myopathy, a 57-year-old male with CGG repeat expansion in LRP12) are shown. Widespread white matter changes with high T2-weighted signals associated with high-intensity signals in the corticomedullary junctions revealed by DWI are shown in the patients with FXTAS, NIID, and OPML. In the patient with FXTAS, cerebral white matter lesions are less prominent than in those with NIID and OPML. T2-weighted high intensity lesions in the middle cerebellar peduncles (MCP sign), a characteristic finding in FXTAS, are also observed in the patient with NIID, whereas slightly high intensity lesions in T2WI are observed in the cerebellar white matter surrounding the deep cerebellar nuclei in the patient with OPML. No abnormal signal intensities or atrophic changes are observed in the patient with OPDM.

FIG. 2 shows a direct identification of repeat expansion mutations by analysis of short reads of whole-genome sequence data. The flow chart shows the scheme for direct identification of repeat expansion mutations employing short reads of whole-genome sequencing data. Step 1: Using TRhist, the present inventors first extract short reads filled with tandem repeats that are overrepresented in patients. Step 2: In the short reads overrepresented in patients, the present inventors observe paired-end reads where both the short reads are filled with tandem repeats as indicated by two gray boxes and those where one of the paired short reads do not contain tandem repeats (nonrepeat reads) as indicated by black boxes. The present inventors then align the nonrepeat reads to the reference genome. As an optional step, the present inventors extract additional paired-end short reads partly filled with tandem repeats (composite boxes with gray and black) and further manually align these short reads and the paired nonrepeat reads (black boxes) to the reference genome. Step 3: The expanded repeats are confirmed by repeat-primed PCR analysis, Southern blot analysis, or long-read sequence analysis.

FIG. 3 shows a summary of the study and clinical overlaps in FXTAS, NIID1, OPML1, OPDM1, and OPMD.

FIG. 4 shows a haplotype analysis of three families with oculopharyngodistal myopathy type 1. Haplotypes were reconstructed using single nucleotide variants genotyped using Affymetrix Genome Wide SNP array 6.0 in three families (F3411, F7758, and F7967). In Families F7758 and F7967, multiple affected individuals were observed, whereas in family F3411onlyoneaffectedindividual (sporadiccase) was observed. In this analysis, the present inventors used hg19 as the reference sequence. First, homozygosity haplotypes were reconstructed (Miyazawa et al. Homozygosity haplotype allows a genome wide search for the autosomal segments shared among patients. Am J Hum Genet80; 1090-1102, (2007)) and shared regions among the three patients were visually confirmed (gray). In addition to SNP array analysis, the present inventors also utilized10X GemCode Technology and compared each haploblock from three families from chr8:105,384,931 to chr8:105,657,322, avoiding genotypes within 10 kb of the boundaries of the haploblock indicated by longranger software. The present inventors selected single nucleotide variants with equal or more than 10 coverages from phased genotypes generated by 10X GemCode Technology. All the phased variants of the three families were matched as indicated by dimgray. These analyses suggested a common founder chromosome among these OPDM1 families.

FIG. 5 shows homologous regions around the CGG repeats in NBPF19. FIG. 5A: Schematic representation of the four highly homologous genes (AC237572.1, NOTCH2, NOTCH2NL, and NBPF14) and NBPF19 are shown. Physical positions in hg38 are indicated. The five genes are located in the pericentric region of chromosome 1. The centromere and a long heterochromatin (1q12) exist between them. Parts of NBPF19, NBPF14, NOTCH2NL, and AC253572.1 have also been recently annotated as NOTCH2NLC, NOTCH2NLB, NOTCH2NLA, and NOTCH2NLR, respectively Widdes, I. T. et al. Cell173, 1356-1369.e22 (2018) and Suzuki, I. K. et al. Cell173, 1370-1384 (2018)]. FIG. 5B: To see sequences with high similarity in these regions, qs core and identity are calculated using BLAT [Kent, W. J. BLAT—the BLAST-like alignment tool. Genome Res.12:646-664 (2002)]. A portion of the NBPF19sequence (chr1:149,370,802-149,410,843 in hg38 that corresponds to 20 kb upstream and 20 kb downstream of the CGG repeats in 5′ UTR of NBPF19) is used as a query. Identities of 99.2%-99.5% are indicated.

FIG. 6 shows Japanese families with NIID enrolled in the present inventor's study.

FIG. 7 shows an identification of CGG repeat expansion mutations in NBPF19 in NIID. FIG. 7A: Number of short reads filled with CGG/CCG tandem repeats in patients with NIID and controls, which were revealed by TRhist using whole genome sequencing data obtained by HiSeq2500. Short reads filled with CGG or CCG repeats were identified in four patients with NIID, whereas no such reads were observed in seven control subjects. FIG. 7B: The CGG/CCG repeat expansions were determined to be located in the 5′ untranslated regions (5′ UTR) of NBPF19, as revealed by alignment of the nonrepeat reads paired with short reads filled with CGG/CCG repeats to the reference genome. Although some of the nonrepeat reads were also aligned to paralogous genes (NBPF14, NOTCH2NL, NOTCH2, and AC253572.1) with enormously high identities with NBPF19 (left and right frames of alignment), the present inventors identified six short reads strongly supporting the alignment to NBPF19 (alignment of one of the six reads is shown in the center frame of aligned nucleotide sequences).

FIG. 8 shows results from TRhist. Data from whole-genome sequence analysis of 150 bp(a) and 126 bp(b) paired-end reads. Only repeat motifs with 3-6 bases that any of the subjects showing more than 9 reads have been observed are shown. Reads filled with CCG(=CGG) repeats are observed in patients with NIID1, OPML1, and OPDM1. NIID1, neuronal intranuclearinclusion disease type 1; OPML1, oculopharyngealmyopathy with leukoencephalopathy type 1; OPDM1, oculopharyngodistal myopathy type 1.

FIG. 9 shows an identification of location of CGG/CCG repeats in families with MID. After short reads filled with CGG/CCG repeats were identified in four patients with NIID, reads paired with reads filled with CGG/CCG repeats were investigated. After trimming using quality score using sickle (version 1.33, https://github.com/najoshi/sickle), reads were visually investigated and mapped to hg38 using BLAT. In patients in F9193, F5804, F9468, and F9785, 6, 7, 13, and 7 reads were mapped to chromosome 1 (boxed with a blue line). In three patients, 3, 2, and 1 nonrepeat reads strongly supported the location of CGG/CCG repeats in NBPF19 (boxed with a red line). In patient 11-6 in F9193, another CGG/CCG repeat was suggested in AFF3 at the fragile site FRA2A located outside the candidate region determined by linkage analysis (data not shown). STR, short tandem repeat.

FIG. 10 shows a characterization of CGG repeat expansion mutations in 5′ UTR of NBPF19 in patients with NIID. FIG. 10A: Schematic representation of NBPF19 indicating the location of CGG repeat expansions. Recently, this region has also been annotated as NOTCH2NLC. The primer set used for repeat-primed PCR (RP-PCR) analysis was designed to detect the expanded CGG repeats on the basis of the unique sequences in NBPF19. FIG. 10B: Representative results of RP-PCR analysis demonstrating CGG repeat expansions in the patients in families F9193 and F6321 (upper and middle panels, respectively). In an unaffected married-in individual, no CGG repeat expansions were detected (lower panel). Experiments were conducted twice with reproducible results. FIG. 10C: CGG repeat expansions in NBPF19 were observed in 26 of the 28 Japanese index patients with NIID (12 probands of the 12 familial cases, 12 of the 14 sporadic cases, and both of the two cases with unavailable family histories). The repeat expansion mutations were also detected in two Malaysian patients. FIG. 10D: Pedigree chart of multiplex families with NIID. Squares and circles indicate males and females, respectively. A diagonal line through a symbol indicates a deceased individual. Affected individuals and those suspected of having the disease are indicated by filled and grey symbols, respectively. The pedigree charts are simplified and scrambled in part including those shown by diamond symbols for confidentiality reason. As shown in the mutation status below the symbols, 11 patients had repeat expansion mutations [exp(+)], whereas three asymptomatic individuals with normal nerve conduction study findings (F6321), three asymptomatic individuals aged >60 years with normal MRI findings (families F9193 and F11393), and two married-in healthy individuals did not [exp(−)]. FIG. 10E: Southern blot analysis revealed expanded alleles in patients with NIID. Probes 1 and 2 were used in the analysis (FIG. 15 and FIG. 16). The lengths of CGG repeat expansions were estimated to range from 270 to 550 bp. Note that lower bands with intense signals represent wild type alleles of NBPF19 and the restriction fragments with the same sizes derived from the other four paralogous genes (AC253572.1, NOTCH2, NOTCH2NL, and NBPF14). Experiments were conducted twice with reproducible results. PBL, genomic DNA extracted from peripheral blood leukocytes; LCL, genomic DNA extracted from lymphoblastoid cell line. FIG. 10F: Distribution of number of CGG repeats in the 5′ UTR of NBPF19. The genomic DNA regions_containing CGG repeats and the flanking sequences were amplified by PCR using an NBPF19-specific primer pair (FIG. 18). The number of CGG repeats were determined_from circular consensus sequencing (CCS) reads. CGG repeats ranged 7-39 repeats in 182 control subjects and there were considerable variations in the repeat configurations. In addition, three SNVs (rs1172135200, rs1258206224, and rs1436954367 designated as “3 SNVs”) were exclusively present in the allele with the repeat motif of (AGG)(CGG)₉(AGG)₃ in 14 control subjects. Another allele carrying rs1258206224 with a configuration of (AGG)(CGG)ₙ(AGG)₂(CGG) were observed in 3 control subjects. The repeat motif of (AGG)(CGG)ₙ(AGG)₂(CGG) was observed in the majority of the alleles and the CGG repeat lengths tended to be larger than those with the repeat motif of (AGG)(CGG)ₙ(AGG)₃.

FIG. 11 shows multiple sequence alignment of a long read, NBPF19, AC253572.1, NOTCH2NL, NBPF14, and NOTCH2. Multiple sequence alignment of a long-read sequence obtained by single-molecule, real-time sequencing, as well as the corresponding regions in NBPF19, AC253572.1, NOTCH2NL, NBPF14, and NOTCH2using ClustalW2 [Larkin, M. A., et al. ClustalW and ClustalX version 2.0. Bioinformatics23, 2947-2948 (2007)]. The five long reads spanning the CGG repeats in NBPF19were subjected to error-correction using Canu (version 1.7) [Koren, S., et al. Canu: scalable and accurate long-read assembly via adaptive k-mer weighting and repeat separation. Genome Res.27, 722-736 (2017)] and then assembled using racon (version 1.3.1) [Vaser, R., et al. Fast and accurate de novo genome assembly from long uncorrected reads. Genome Res.27, 737-746 (2017)]. CGG repeat expansions were shown by boxes in FIG. 11B and FIG. 11C. An NBPF19-specific insertion of Alu sequence was shown by boxes in FIG. 11K and FIG. 11L, which confirmed that the expanded CGG repeats were located in NBPF19. One of the primer sequence (NBPF19-R, FIG. 13) for repeat-primed PCR analysis (shown by a box in FIG. 11D) and a primer pair (pGEX3′-NBPF19-6F and NBPF19-5R2, FIG. 17) for fragment analysis (shown by boxes in FIG. 11A and FIG. 11E) were designed to avoid nonspecific amplification.

FIG. 12 shows raw and corrected long reads. Rows with white background and those with grey background show read names, properties of reads and nucleotide sequences before error correction and those after error correction by Canu, respectively.

FIG. 13 shows primer sequences used for repeat-primed PCR analysis

FIG. 14 shows primer sequences used for the repeat-primed PCR analysis of FMR1. The present inventors used deaza-dGTPin place of dGTP. PCR reaction was conducted as follows; initial denaturation at 94° C. for 1 min, followed by 30 cycles of 94° C. for 30 s, 60° C. for 30 s, and 72° C. for 80 s or slow down PCR protocol shown in present disclosure. GCII buffer was obtained from TaKaRa (TaKaRaLA taq with GC buffer).

FIG. 15 shows primer sequences used for preparation of template and probes for Southern blot analysis. Genomic DNA segments flanking the CGG repeats were amplified using the primer pairs (NBPF19_1/NBPF19_4, NBPF19_2 aF2/NBPF19_2cF3, 2107F/3052R, and 2243F/2995R) and subcloned into plasmids. Probes for Southern blot hybridization analysis were prepared by digoxigenin(DIG) labeling using primer pairs (NBPF19_1/NBPF19_1R for Probe 1 and NBPF19_4F/NBPF19_4 for Probe 2, NBPF19_2 aF2/NBPF19_2 aR2 for Probe 3, NBPF19_2 bF2/NBPF19_2 bR2 for Probe 4, and NBPF19_2cF2/NBPF19_2 cR2 for Probe 5 [NBPF19], 2107F/2531R for Probe 6 [LOC642361/NUTM2B-AS1], and 2243F/2562R for Probe 7 and 2538F/2995R for Probe 8 [LRP12]).

FIG. 16 shows an intergenerational instability of the CGG repeats in NBPF19. FIG. 16A: SacI/NheI digestion sites around the CGG repeats in the 5′ UTR of NBPF19 are shown. An Alu sequence (starred) on the downstream of the CGG repeats is absent in the other 4 highly homologous genes (AC253572.1, NOTCH2, NOTCH2NL, and NBPF14). This enabled the present inventors to distinguish the NBPF19 alleles from other highly homologous genes in Southern blot analysis using NheI-digested genomic DNA (gDNA). Restriction fragments generated from NOTCH2, AC253572.1, NBPF14, and NOTCH2NL are estimated to be 2,696 bp, 2,691 bp, 2,696 bp, and 2,707 bp, respectively, whereas that from NBPF19 is estimated to be 3,009 bp basedonhg38. FIGS. 16B and 16C: Southern blot analysis of parent-offspring pairs in the branches of F6321 using NheI-digested gDNA, where the present inventors use probes 1-5 to enhance the signal intensity of target bands. White arrows indicate fragments derived from the 4 genes (NOTCH2, AC253572.1, NBPF14, and NOTCH2NL) that do not carry the Alu sequence designated by a star in (a) and gray arrows indicate wild typeNBPF19 alleles that carry the Alu sequence. Black arrows indicate NBPF19 alleles with expanded CGG repeats. The results showed that the sizes of the CGG repeats in NBPF19 become larger in the successive generations. The parent indicated by a gray symbol in (b) only showed abnormalities in the nerve conduction study.

FIG. 17 shows primer sequences used for the fragment analysis in controls subjects. PCR reaction was conducted as follows; initial denaturation at 98° C. for 1 min, followed by 35 cycles of 98° C. for 10 sec, 58° C. for 30 sec, and 68° C. for 30 sec for NBPF19, initial denaturation at 95° C. for 1 min, followed by 30 cycles of 94° C. for 30 s, 50° C. for 30 s, and 72° C. for 60 s for LOC642361/NUTM2B-AS1, and .initial denaturation of 98° C. for 1 min, followed by 35 cycles of 98° C. for 10 sec, 60° C. for 30 sec, and 68° C. for 30 sec for LRP12. GCII buffer was obtained from TaKaRa (TaKaRaLA taq with GC buffer).

FIG. 18 shows primer sequences and barcode sequences used for the circular consensus sequencing (CCS) analysis using a SMRT sequencer. Each forward and reverse primers contained 16-mer barcodes as shown below. PCR reaction was conducted as follows; initial denaturation at 98° C. for 1 min, followed by 35 cycles of 98° C. for 10 sec, 58° C. for 30 sec, and 68° C. for 30 sec. GCII buffer was obtained from TaKaRa (TaKaRaLA taq with GC buffer).

FIG. 19 shows repeat configurations of CGG and flanking repeats in NBPF19 in control subjects as revealed by CCS analysis. FIG. 19A: The CGG and flanking repeats in the 5′ UTR of NBPF19 is (AGG)(CGG)₉(AGG)₂(CGG) in the reference sequence (hg38). To determine the number of repeat units, repeat configurations and single nucleotide variants in the flanking sequences, circular consensus sequencing (CCS) analysis was performed for pooled barcoded PCR products from 182 control subjects. CCS reads were confirmed to have NBPF19-specific sequence shown by a underline. FIG. 19B: The present inventors observed 11 repeat configurations and single nucleotide variants (SNVs) in the flanking sequences in NBPF19. One allele carrying three SNVs (rs1172135200, rs1436954367, and rs1376391857) in the flanking sequences, all of which carried a configuration (AGG)(CGG)₉(AGG)₃, and another allele carrying rs1258206224 with a configuration of (AGG)(CGG)ₙ(AGG)₂(CGG) were observed in 14 and 3 controls, respectively. On the basis of these observations, distribution of number of the CGG repeat unit (shown by “n”) was determined (FIG. 30).

FIG. 20 shows a frequency distribution of repeat sizes in NBPF19 in 1,000 control subjects as revealed by fragment analysis. FIG. 20A: Frequency distribution of repeat sizes of the CGG repeats and the flanking variable repeat sequences in NBPF19 of 1,000 control subjects was determined by fragment analysis of PCR products obtained using NBPF19-specific primer pair (pGEX3′-NBPF19-6F and NBPF19-5R2). In the reference sequence (hg38), the repeat size is 13 repeat units, namely, (AGG)(CGG)₉(AGG)₂(CGG). FIG. 20B: Multiple sequence alignment of the five homologous sequences (NBPF19, AC253572.1, NOTCH2NL, NBPF14, and NOTCH2) using Clustal W2 is shown. Variable repeat sequences including CGG repeats are shown below a line. In the fragment analysis, repeat sizes were determined as the lengths in repeat units between the flanking non-variable sequences (shown below dotted lines). Primers used in the analysis are shown by arrows (pGEX3′-NBPF19-6F and NBPF19-5R2). Numbers shown in the figures indicate relative distances from 149,390,308 (NBPF19), 120,723,618 (AC253572.1), 146,229,332 (NOTCH2NL), 148,680,074 (NBPF14), and 120,069,958 (NOTCH2).

FIG. 21 shows inter-pulse durations (IPDs) in CGG sites examined by SMRT sequencing. The present inventors first created a reference IPD set for the hypomethylated CGGs and hypermethylated CGGs using whole-genome bisulfite sequencing data and PacBio Sequel sequencing data (both obtained from the same individual). The reference benchmark set had 303 hypomethylated CGG repeat regions with 1,220 Cp Gs and 14 hypermethylated regions with 59 CpGs. The present inventors observed a significant difference in IPD statistics (on cytosine sites of CGG) between the methylated (n=59) and unmethylated (n=1,220) CpG sites (*p=3.3×10⁻¹⁶, one-sided) using Mann-Whitney U test, demonstrating that IPD is informative in inferring CpG methylation status of CGG repeats. The present inventors next examined whether the expanded CGG repeat in the 5′ UTR of NBPF19 was similar to hypomethylated CGG repeats or hypermethylated CGG repeats in terms of IPD statistics of CpG sites, and the present inventors checked the null hypothesis of independence of IPD statistics using Mann-Whitney U test. The present inventors found that the IPD distribution on cytosine sites of the expanded CGG repeat in the 5′ UTR of NBPF19 (n=60) was similar to that of hypermethylated CGG repeats (n=59) (***p=0.35, two-sided test) but was significantly dissimilar to that of hypomethylated CGG repeats (n=1,220) (**p=1.6×10⁻⁴, one-sidedtest), showing that the expanded CGG repeat in the 5′ UTR of NBPF19 was regionally hypermethylated as a whole.

FIG. 22 shows an expression levels of NBPF19 in brains examined by RNA-seq. FIG. 22A: There are 4 positions in noncoding exon 1 of NBPF19 whose sequences are unique to NBPF19 among the five homologous sequences in AC253572.1, NOTCH2, NOTCH2NL, NBPF19, and NBPF14. Physical positions in hg38 are shown. From RNA-seq data from 3 patients with NIID and 8 control subjects (occipital lobe), read per million mapped reads of the positions were calculated. Because one of the position is just downstream of the CGG repeats (chr1:149,390,838 in hg38), which made precise alignment difficult, the present inventors did not calculate coverages of the position. FIG. 22B: Expression levels of NBPF19 the present inventors reassessed using read per million mapped reads in the three positions as described above. The present inventors did not see any statistically significant differences between NIID (n=3) and control subjects (n=8, Wilcoxson rank sum tests, two-sided). The data are shown as means and standard errors of means.

FIG. 23 shows an identification of CGG repeat expansions in LOC642361/NUTM2B-AS1 in a family with oculopharyngeal myopathy with leukoencephalopathy (OPML). FIG. 23A: Schematic representation of exons of LOC642361 and NUTM2B-AS1, both of which encode noncoding RNA. The directions of the transcription are indicated by arrows. The primer set used for repeat-primed PCR (RP-PCR) analysis is designed to detect expanded CGG repeats (a line and arrows). FIG. 23B: Representative results of RP-PCR analysis showing CGG repeat expansions in patients in the family F5305 (upper and middle panels). In an unaffected married-in individual, no CGG repeat expansions were detected (lower panel). Experiments were conducted twice with reproducible results. FIG. 23C: Pedigree chart of the family with OPML. Squares and circles indicate males and females, respectively. A diagonal line through a symbol indicates a deceased individual. Affected individuals are indicated by filled symbols. The pedigree charts are simplified for confidentiality reason. As shown in the mutation status below the symbols, four patients had repeat expansion mutations [exp(+)], whereas seven unaffected individuals including three married-in individuals did not [exp(−)]. FIG. 23D: Frequency distribution of repeat units of CGG repeats of 1,000 control subjects in LOC642361/NUTM2B-AS1 as revealed by fragment analysis is shown. LOC642361/NUTM2B-AS1-specific primers were used for amplification. In the reference sequence (hg38), (CGG)₆ is registered.

FIG. 24 shows short reads indicating CGG repeat expansion in LOC642361/NUTM2B-AS1. FIG. 24A: Nine nonrepeat reads paired with reads filled with CGG/CCG repeats were identified in patient 111-5 in F5305. Seven of the nine reads were mapped to the LOC642361/NUTM2B-AS1 region best by BLAT. STR, short tandem repeat. FIG. 24B: Alignment of nonrepeat reads paired with reads filled with CGG/CCG repeats indicates that CGG repeat expansion is located in LOC642361/NUTM2B-AS1. Reads are shown in the same strand as the direction of transcription of LOC642361. Homologous sequences of LOC642361/NUTM2B-AS land mismatches among them are shown in red squares.

FIG. 25 shows a linkage analysis of family (F5305) with OPML. Parametric linkage analysis results of family with OPML (F5305, FIG. 23) for all chromosomes (a) and candidate regions (b) are shown. Chromosome 10 is the only chromosome that shows LOD score of above 1. Boundary markers with physical positions in hg38 are indicated below. The locus of LOC642361/NUTM2B-AS1 is indicated by an arrow.

FIG. 26 shows a bidirectional transcription of CGG/CCG repeats in LOC642361/NUTM2B-AS1. Stranded RNA-seq data of a control brain and two control muscles using random primers in reverse transcription reactions are shown. Short reads are aligned to the reference sequence (hg38) using STAR [Dobin, A., et al. STAR: ultrafast universal RNA-seq aligner. Bioinformatics29, 15-21 (2013)]. Reads are divided into two files according to the direction of transcription. Only reads with mapping quality equal or more than 5 are shown using the Integrative Genomic Viewer [Robinson, J. T., et al. Integrative Genomic Viewer. Nat. Biotechnol. 29, 24-26 (2011)]. FIG. 26A: The CGG/CCG repeats in LOC642361/NUTM2B-AS1 were bidirectionally transcribed, although coverages at the CGG/CCG repeat were underrepresented presumably owing to its high GC content. FIG. 26B: No signals suggesting bidirectional transcription were observed in the CGG repeats in 5′ UTR of NBPF19, although a mapping problem remains in the locus considering other highly homologous sequences. FIG. 26C: Most of the reads in exon 1 of LRP12 were sense reads, whereas only trivial antisense reads were observed.

FIG. 27 shows a homologous regions of CGG repeats in LOC642361/NUTM2B-AS1. The regions of CGG repeats in LOC642361/NUTM2B-AS1 have two homologous sequences with high similarity in the reference genome (hg38). Identity and qs core are calculated using BLAT. The sequence (chr10:79,825,306-79,827,410) that corresponds to 1 kb upstream and downstream of the CGG repeat in LOC642361/NUTM2B-AS1 is used as a query.

FIG. 28 shows multiple sequence alignments of homologous genes of LOC642361/NUTM2B-AS1. Multiple sequence alignment of sequence around the CGG/CCG repeats in LOC642361/NUTM2B-AS1 with homologous sequences of LINC00863/NUTM2A-AS1 (chromosome 10) and FLJ22063/AMMECR1L (chromosome 2) using ClustalW2. Sequences are derived from hg38. The position of CGG repeat expansion mutations is shown in a box. The primer sequence (L00642361-R2, FIG. 13) for repeat-primed PCR analysis (shown by a lower arrow in FIG. 28B) and a primer pair (LOC642361_PCR-F3 and pGEX3′-LOC642361_PCR-R, FIG. 17) for fragment analysis (shown by an arrow in FIG. 28A and shown by a upper arrow in FIG. 28B) were designed to avoid nonspecific amplification.

FIG. 29 shows a southern blot analysis of LOC642361/NUTM2B-AS1. FIG. 29A: Southern blot analysis was performed using probes targeting flanking regions of the CGG repeats in LOC642361/NUTM2B-AS1in chromosome 10. The probes were also predicted to hybridize to the other two similar sequences (LINC00863/NUTM2A-AS1in chromosome 10 and FLJ22063/AMMECR1Lin chromosome 2). Predicted fragment sizes based on hg38 are 1.4 kb (LOC642361/NUTM2B-AS1), 1.4 kb (LINC0863/NUTM2A-AS1), and 1.1 kb (FLJ22063/AMMECR1L). Strong somatic instability of the CGG repeats was observed in genomic DNAs from peripheral blood leukocytes (PBL). The experiment was conducted once. FIG. 29B: An expanded allele of 2.1 kb (corresponding to 700 repeat units) was observed in genomic DNA from lymphoblastoid cell line of patient 111-3 of family F5305. NC: normal control. The experiments were conducted twice with similar results.

FIG. 30 shows an identification of CGG repeat expansions in LRP12 in families with oculopharyngodistal myopathy (OPDM). FIG. 30A: Schematic representation of exons of LRP12. The CGG repeat expansion is located in the 5′ untranslated region (5′ UTR). The primer set used for repeat-primed PCR (RP-PCR) analysis is designed to detect expanded CGG repeats (a line and arrows). FIG. 30B: Representative results of RP-PCR analysis indicating CGG repeat expansions in patients in the families F7967 and F3411 (upper and middle panes). In an unaffected control, no CGG repeat expansions were detected (lower panel). Experiments were conducted twice with reproducible results. FIG. 30C: Pedigree charts of families with OPDM. Squares and circles indicate males and females, respectively. A diagonal line through a symbol indicates a deceased individual. Affected individuals are indicated by filled symbols. The pedigree charts are simplified for confidentiality reason. As shown in the mutation status below the symbols, three affected individuals had repeat expansion mutations [exp(+)], whereas the unaffected individual did not [exp(−)]. FIG. 30D: The CGG repeat expansions in LRP12 were identified in 38.2% of patients with supporting histopathological findings of rimmed vacuoles (RVs) and 16.7% of patients with unavailable histopathological findings. No CGG repeat expansions in LRP12 were found in patients with similar clinical presentations but without RVs in biopsied muscle specimens. FIG. 30E: Frequency distribution of repeat units of CGG repeats of 1,000 control subjects in LRP12 as revealed by fragment analysis is shown. The repeat configuration in the reference sequence (hg38) is (CGG)₉(CGT)(CGG)(CGT)₂. The number of repeat units for this allele was defined as 13 in this analysis.

FIG. 31 shows short reads indicating CGG repeat expansion in LRP12. FIG. 31A: Three nonrepeat reads paired with reads filled with CGG/CCG repeats were identified in patient III-1 in F7967. All the three reads were mapped to the LRP12 region by BLAT. STR, short tandem repeat. FIG. 31B: Alignment of nonrepeat reads paired with reads filled with CGG/CCG repeats indicates that CGG repeat expansion is located in 5′ UTR of LRP12. Reads are shown in the same strand as the direction of transcription of LRP12.

FIG. 32 shows a southern blot analysis of patients with oculopharyngodistal myopathy and controls. FIG. 32A: Southern blot analysis of patients with OPDM1. In genomic DNAs from lymphoblastoid cell lines (LCLs), multiple bands presumably derived from somatic instabilities (gray arrows) were observed, whereas single expanded bands (230 and 380 bp, black arrows) were observed in genomic DNAs from peripheral blood leukocytes (PBL). This experiment was conducted once. FIG. 32B: In the two controls who had the longest repeats as suggested by repeat-primed PCR analysis, whose ages at blood sampling were 63 years and 25 years, the expanded CGG repeat sizes exceeded 300 bp (black and gray arrows) and multiple bands were observed in genomic DNA from LCL (gray arrows). This experiment was conducted once. Exp+, carrier of expansion; exp−, noncarrier of expansions.

FIG. 33 shows clinical characteristics of the family (F5305) with oculopharyngeal myopathy with leukoencephalopathy (OPML). Abbreviation: y/o, years old; ND, not described; N/A: not applicable; MMSE, Mini Mental State Examination; HDS R, The Revised Hasegawa dementia scale; WAIS R, Wechsler Adult Intelligence Scale revised; PIQ, performance intelligence Quotient; VIQ, verbal intelligence quotient; TIQ, total intelligence quotient.

### DESCRIPTION OF EMBODIMENTS

Unstable tandem repeat expansions have been shown to be involved in a wide variety of neurological diseases. Given a rapidly increasing number of diseases belonging to this group, it is expected that many more diseases await identification of causative genes. Availability of massively parallel short-read sequencers has dramatically accelerated the search for causative genes including the de novo sequencing research paradigm. Since there remain difficulties in the detection of expanded tandem repeats with short-read sequencers, development of straightforward and efficient strategies for directly identifying expanded tandem repeats is expected to dramatically accelerate gene discoveries.

As the first candidate disease for direct search for expanded tandem repeat mutations, the present inventors selected neuronal intranuclear inclusion disease (NIID, MIM603472, https://omim.org/) in the present inventor's study. NIID is a neurodegenerative disease characterized clinically by various combinations of cognitive decline, parkinsonism, cerebellar ataxia and peripheral neuropathy, and neuropathologically by eosinophilic hyaline intranuclear inclusions in the central and peripheral nervous systems as well as in other tissues including cardiovascular, digestive, and urogenital organs. The age at onset ranges from infancy to late adulthood. Although an autosomal dominant mode of inheritance has been assumed, about two-thirds of cases have been reported to be sporadic. Recently, characteristic magnetic resonance imaging (MRI) findings including high-intensity signals in diffusion-weighted imaging (DWI) in the corticomedullary junction and eosinophilic intranuclear inclusions observed in skin biopsy have been described as useful diagnostic hallmarks for NIID. Following these reports, a rapidly increasing number of MID cases, particularly those with late adult onset, have recently been reported.

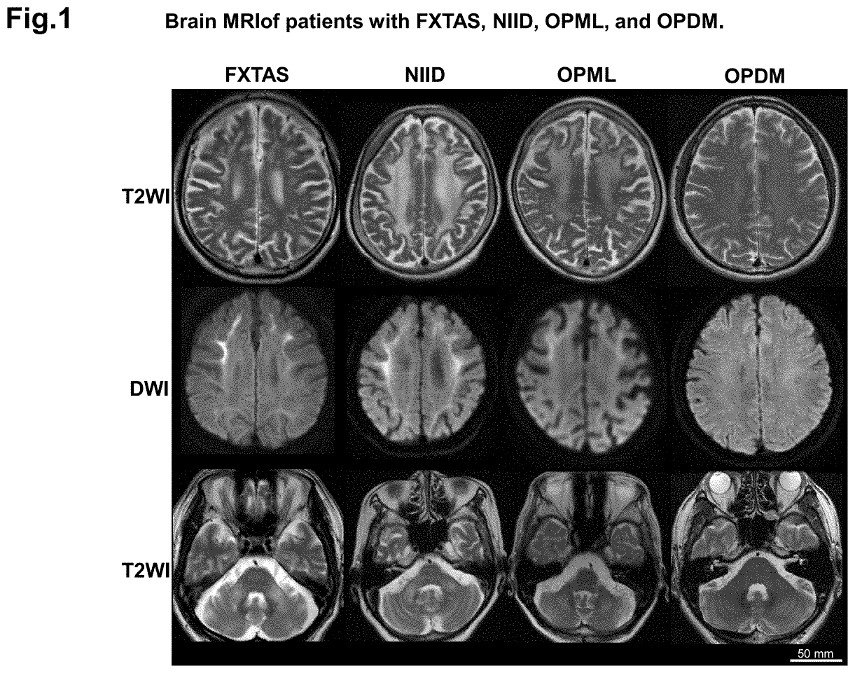

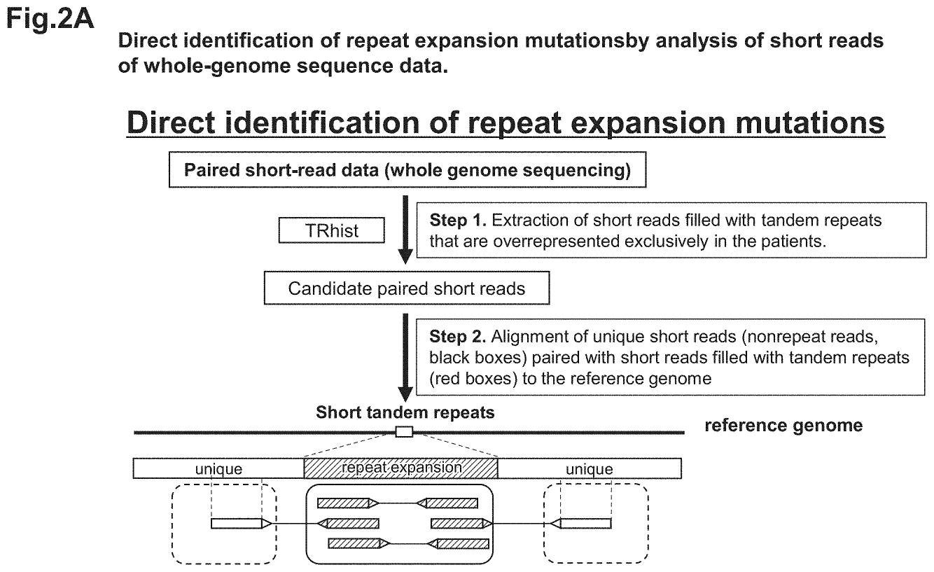

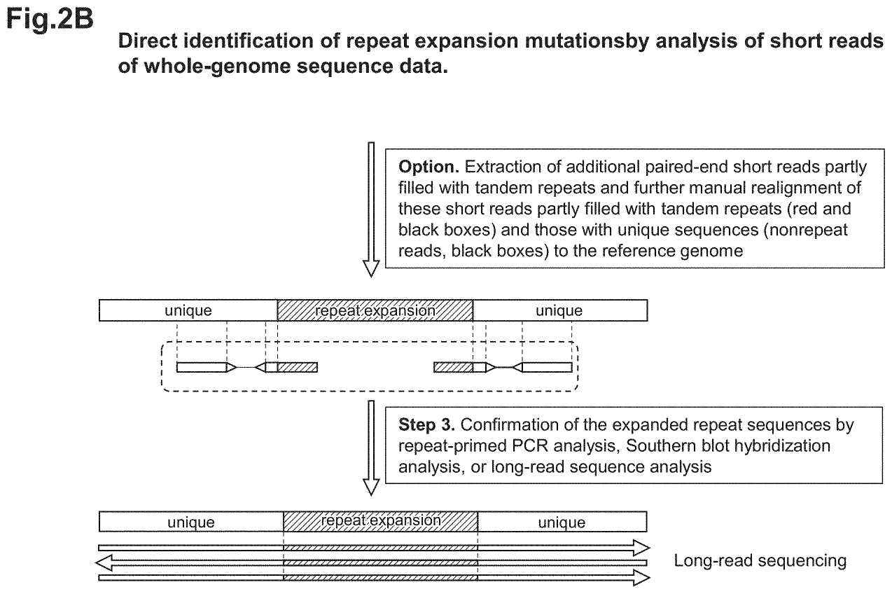

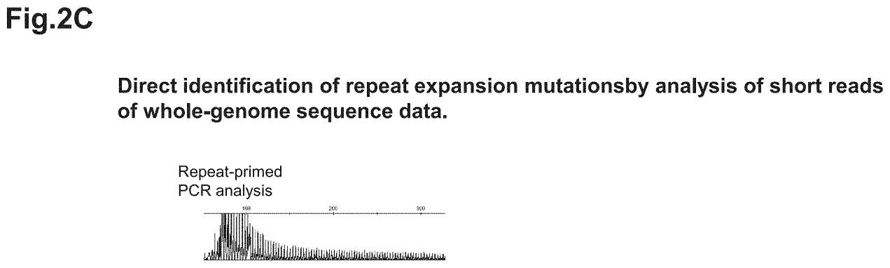

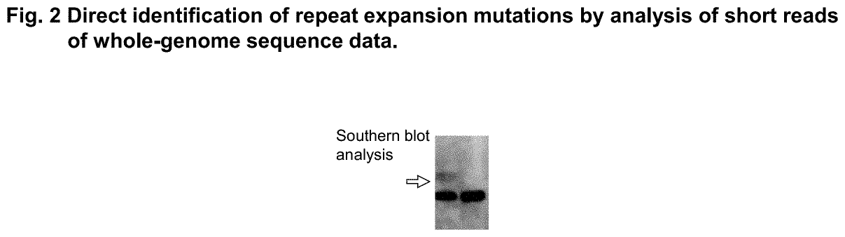

Inspired by the striking similarity of MRI findings between NIID and fragile X tremor/ataxia syndrome (FXTAS, MIM300623), including T2-hyperintensity areas in the middle cerebellar peduncles (MCP sign) and high-intensity signals on DWI in the corticomedullary junction that are also occasionally observed in FXTAS (FIG. 1), and the presence of eosinophilic intranuclear inclusions observed in the two diseases, the present inventors hypothesized that NIID shares a common molecular basis with FXTAS, a disease caused by mildly expanded CGG repeats (premutation) in the 5′ untranslated region (UTR) of FMR1 with repeat units of 55-200. To explore the possibility of expanded CGG repeats in NIID, the present inventors devised the direct search strategy (FIG. 2) to efficiently identify expanded repeats in the human genome using TRhist, which produces histograms of short reads filled with tandem repeats. Employing TRhist, the present inventors indeed identified accumulation of short reads filled with CGG repeats in the 5′ UTR of NBPF19 in NIID in this present inventor's study.

Prompted by the similarity in the clinical and neuroimaging findings with NIID, the present inventors further identified similar noncoding CGG repeat expansions in two other diseases, oculopharyngeal myopathy with leukoencephalopathy (OPML) and oculopharyngodistal myopathy (OPDM, MIM164310), in LOC642361/NUTM2B-AS1 and LRP12, respectively. Taken together with the present inventor's previous findings, this present study further expands the concept that noncoding repeat expansion mutations involving the same repeat motifs, along with tissues where the genes are transcribed, lead to diseases with similar or overlapping clinical presentations, and provides a new straightforward approach to discover repeat expansion mutations underlying a wide variety of diseases.

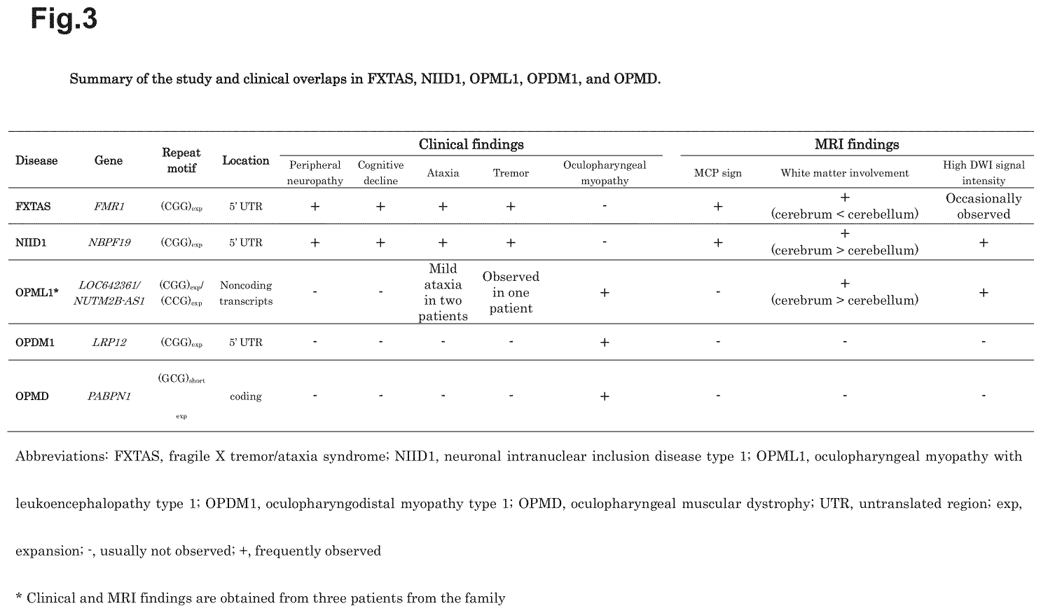

Here, the present inventors identified noncoding CGG repeat expansions in the three genes, NBPF19, L00642361, and LRP12, as the disease-causing mutations for NIID, OPML and OPDM, respectively (FIG. 3). The present inventors herein designate the diseases with the repeat expansions in NBPF19, L00642361, and LRP12 as NIID1, OPML1, and OPDM1, respectively.

Including FXTAS and OPMD, these five diseases are caused by expansions involving the same repeat motif. Although the clinical presentations of FXTAS, NIID, OPML, OPDM, and OPMD are distinct, there are considerable overlaps among these diseases (FIG. 3), suggesting that transcribed expanded CGG repeats are commonly involved in the development of these diseases, irrespective of the genes where the expanded repeats are located. The present inventors have recently discovered that noncoding TTTCA repeat expansions in three genes cause benign adult familial myoclonic epilepsies (BAFME1 [MIM601068], BAFME6 [MIM618074], and BAFME7 [MIM618075]). Thus, the findings that the same expanded repeat motifs located in different genes lead to overlapping clinical spectra of diseases further expand the knowledge on the noncoding repeat expansion diseases. Although the tissue expression patterns of causative genes may modify their clinical presentations, what factors determine the distinct clinical characteristics among FXTAS, NIID1, OPML1, and OPDM1 remain to be further explored.

Although the frequency is very low, CGG repeat expansions in LRP12 were observed in a limited number of control subjects (0.2%). Regarding CGG repeat expansions in FMR1, 0.21% of males in controls had expansions (55-200 repeat units) in the United States. In frontotemporal lobar degeneration/amyotrophic lateral sclerosis (FTLD/ALS) caused by GGGGCC repeat expansions in C9orf72 [MIM105550], 0.15% of controls in the United Kingdom and 0.4% of controls in Finland have repeat expansions. Thus, rare occurrence of repeat expansions in controls seems to be common findings in noncoding repeat expansion diseases. Detailed investigations of the structures of expanded repeats and the haplotypes flanking the expanded repeats of the patients and controls may provide an insight into the mechanisms underlying the phenomenon.

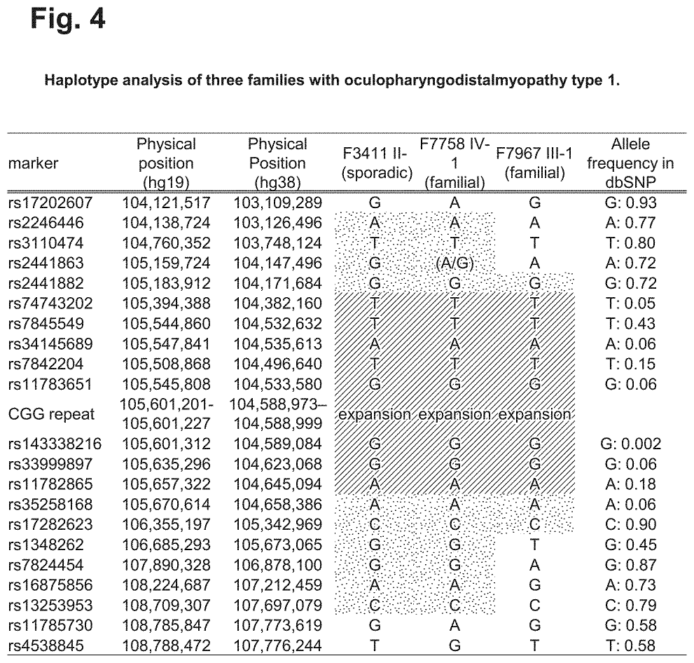

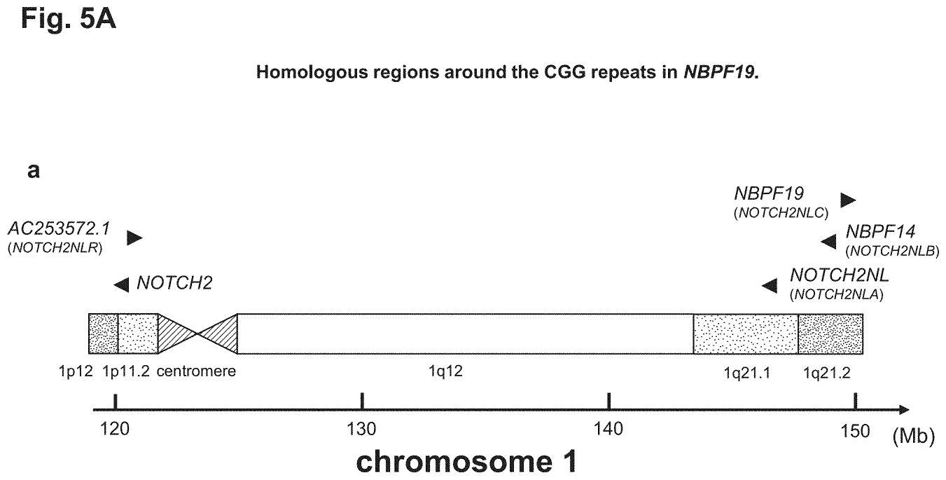

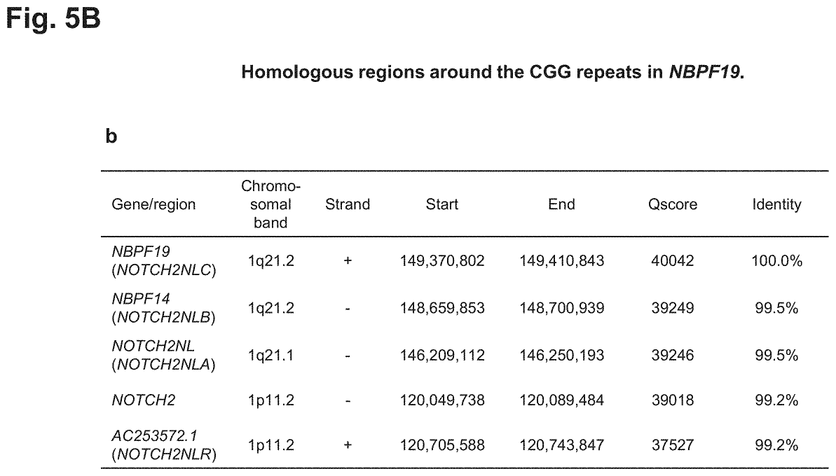

Founder haplotypes have been identified in many repeat expansion diseases. Haplotype analysis in families with OPDM revealed a shared haplotype, suggesting a founder effect (FIG. 4). Because of the sequences with enormously high identities in the NBPF19 locus to the paralogous genes and the long heterochromatin (1q12) next to the locus (FIG. 5), the present inventors were unable to unambiguously determine the haplotypes of families with NIID.

Of note, both FXTAS and C9ORF72-linked FTLD/ALS are well documented in sporadic cases. Family histories were documented only in 50% of Japanese families with NIID1 and 41% of patients with OPDM1 in the present case series, suggesting that the present inventors need to pay attention not only to familial cases but also to sporadic cases presenting with similar clinical features. Furthermore, diversities in clinical presentations and ages at onset have also been observed in these diseases. Although the mechanisms are as yet unknown, dynamic instability of noncoding repeat expansions among tissues as well as in germlines may underlie these phenomena.

In the present inventor's case series, 7.1% of Japanese NIID patients and 61.8% of OPDM patients with supporting pathological findings of biopsied tissues did not have CGG repeat expansion mutations in NBPF19 and LRP12, respectively. Thus, there remains a possibility of genetic heterogeneity in these diseases. Further search for CGG repeat expansions located in other loci or repeat expansions involving similar repeat motifs will be a feasible approach.

Analysis of methylation status of expanded CGG repeats in a patient with NIID using SMRT sequence reads showed a tendency of hypermethylation of CGG repeats. The present inventors did not, however, detect statistically significant decrease of NBPF19 transcripts, indicating that expanded alleles are not fully silenced. In addition, Fiddes et al. reported that NBPF19/NOTCH2NLC (which they call NOTCH2NLC-like paratype) had variable copy numbers with the frequency of 0, 1, and 2 copies being 0.4%, 6%, and 92%, respectively, indicating that haploinsufficiency of NBPF19 unlikely causes NIID.

In FXTAS, ubiquitinated inclusions have been shown in brains and non-neuronal tissues. After the discovery of repeat-associated non-ATG-initiated (RAN) translation, RAN proteins have been revealed to be a component of the ubiquitinated inclusions in FXTAS. NIID and OPDM are pathologically characterized by intranuclear inclusions and tubulofilamentous inclusions, respectively. Thus, it is conceivable to postulate that these inclusions observed in NIID and OPDM contain RAN proteins, although it awaits confirmation. In contrast, routine histopathological examinations of biopsied muscle from the two patients (I11-3 and 111-5 in F5305) did not reveal inclusions in OMPL1. RNA-mediated toxicity through the sequestration of RNA-binding proteins that recognize expanded CGG repeats may also be variably involved in these diseases.

Identification of disease-causing repeat expansions has been accomplished usually by laborious classical positional cloning approaches. As shown in the present disclosure, the present inventors used TRhist to directly detect repeat expansions from short-read next-generation sequencing data and discovered the causative genes by alignment of nonrepeat reads of the paired short reads to the reference genome. Among the recently developed programs targeting repeat expansions from the short-read data, an advantage of TRhist is its ability to detect insertions of any kind of expanded repeats including those containing novel repeat motifs that are not present in the reference genome. Since the present inventor's strategy (FIG. 2) does not require prior linkage analysis, it can be applicable to families with variable penetrances and even to sporadic patients without family histories. Availability of single-molecule long-read sequencers should further complement the search for disease-causing repeat expansions employing currently standard short-read next-generation sequencers. Considering that there are ˜80,000 microsatellites with 3-6 bases in introns of the human genome that could potentially undergo expansion, which by far exceed the number of 20,000 protein-coding and 22,000 noncoding genes (Ensembl, https://www.ensembl.org/), the search for noncoding repeat expansions is expected to further expand the present inventor's knowledge regarding the genetic architecture of a wide variety of diseases or traits.

In conclusion, the present inventors identified noncoding CGG repeat expansions as the causes of NIID1, OPML1, and OPDM1. These findings expand the present inventor7s insights into the molecular basis of these diseases and further emphasize the importance of noncoding repeat expansions in a wide variety of neurological diseases.

Based on the above findings by the present inventors, a method for determining, diagnosing, or aiding to diagnose a neuromuscular disease accompanied with a repeat expansion of CGG in a nucleic acid in a subject according to the embodiment of the present invention comprises detecting a repeat expansion of CGG or a complementary sequence thereof in a nucleic acid sample from the subject. Examples of the neuromuscular disease accompanied with the repeat expansion of CGG are neuronal intranuclear inclusion disease, (NIID) oculopharyngodistal myopathy (OPDM), and oculopharyngeal myopathy with leukoencephalopathy (OPML). Clinically, most cases of MID present as a multisystem neurodegenerative process beginning in the second decade and progressing to death in 10 to 20 years. Neurological signs and symptoms vary widely, but usually include ataxia, extra-pyramidal signs such as tremor, lower motor neuron findings such as absent deep tendon reflexes, weakness, muscle wasting, foot deformities and less apparent behavioral or cognitive difficulties. Reported adult-onset cases are characterized by dementia and may represent different clinical presentations. In the present disclosure, the neuromuscular disease excludes fragile X syndrome, fragile X tremor ataxia syndrome (FXTAS), and oculopharyngeal muscular dystrophy.

The presence of the repeat expansion in the nucleic acid sample indicates that the subject has the neuromuscular disease or is at risk of having the neuromuscular disease. The method can be used for determining whether the subject has or is at risk of having the neuromuscular disease.

The subject is a human being or a non-human animal. The subject may be a patient who may have the neuromuscular disease. The nucleic acid sample may be collected from the subject prior to the detection of the repeat expansion. The nucleic acid sample may be collected from a cell from the subject. The cell may be leukocyte, lymphocyte, monocyte, erythroblast, hematopoietic stem cell, or hematopoietic progenitor cell. The method may be carried out in vivo. The nucleic acid sample may be DNA, such as chromosome DNA, or alternatively, the nucleic acid sample may be RNA. The repeat expansion of CGG may be in an intron of any gene from the subject.

In the case where the neuromuscular disease is neuronal intranuclear inclusion disease, the repeat expansion of CGG may be in 5′ untranslated region of NBPF19 gene. In the case where the neuromuscular disease is neuronal intranuclear inclusion disease, the repeat expansion may be greater than 70 repeats, greater than 75 repeats, greater than 80 repeats, greater than 85 repeats, or greater than 90 repeats. In the case where the neuromuscular disease is neuronal intranuclear inclusion disease, the size of the expanded CGG may be greater than 210 base pairs, greater than 225 base pairs, greater than 240 base pairs, greater than 255 base pairs, or 270 base pairs.

In the case where the neuromuscular disease is oculopharyngodistal myopathy, the repeat expansion of CGG may be in 5′ untranslated region of LRP12 gene. In the case where the neuromuscular disease is oculopharyngodistal myopathy, the repeat expansion may be greater than 70 repeats, greater than 75 repeats, greater than 80 repeats, greater than 85 repeats, or greater than 90 repeats. In the case where the neuromuscular disease is oculopharyngodistal myopathy, the size of the expanded CGG may be greater than may be greater than 210 base pairs, greater than 225 base pairs, greater than 240 base pairs, greater than 255 base pairs, or 270 base pairs.

In the case where the neuromuscular disease is oculopharyngeal myopathy with leukoencephalopathy, the repeat expansion of CGG may be in 5′ untranslated region of LOC642361 gene and/or NUTM2B-AS1 gene. In the case where the neuromuscular disease is oculopharyngeal myopathy with leukoencephalopathy, the repeat expansion may be greater than 70 repeats, greater than 75 repeats, greater than 80 repeats, greater than 85 repeats, or greater than 90 repeats. In the case where the neuromuscular disease is oculopharyngeal myopathy with leukoencephalopathy, the size of the expanded CGG may be greater than 210 base pairs, greater than 225 base pairs, greater than 240 base pairs, greater than 255 base pairs, or 270 base pairs.

A kit for determining or diagnosing a neuromuscular disease accompanied with a repeat expansion of CGG in a nucleic acid in a subject according to the embodiment of the present invention comprises a nucleic acid reagent configured to detect a repeat expansion of CGG or a complementary sequence thereof in a nucleic acid sample from the subject. Examples of the neuromuscular disease are neuronal intranuclear inclusion disease, oculopharyngodistal myopathy, and oculopharyngeal myopathy with leukoencephalopathy.

The kit can be used for the method for determining or diagnosing the neuromuscular disease in the subject according to the embodiment of the present invention. The kit may be used in vivo.

The nucleic acid reagent may comprise a PCR primer configured to detect the repeat expansion of CGG or the complementary sequence thereof. The PCR primer may comprise a complementary sequence of CGG or a complementary sequence thereof.

The PCR may be a repeat-primed PCR and a long-range PCR. The repeat-primed PCR and the long-range PCR can detect the repeat expansion. An application on the repeat-primed PCR is described in Neuron 72, 257-268, Oct. 20, 2011. In the repeat-primed PCR, nucleic acids are amplified between a forward primer and a reverse primer at an initial stage. Since the concentration of the forward primer is low, the forward primer is wasted. Thereafter, the nucleic acids are amplified between an anchor primer and the reverse primer. If the anchor primer does not present, a repeat sequence is randomly annealed. In such case, only short PCR products are produced, and it is difficult to detect a repeat expansion. If the anchor primer presents, PCR products are produced between the anchor primer and the reverse primer so that they reflect the distribution of PCR products produced at the initial stage by the annealing of the forward primer. A comb-like distribution of the PCR product can be obtained. It should be noted that the anchor primer is not limited to any specific sequence.

Alternatively, the nucleic acid reagent in the kit may comprise a hybridization probe configured to detect the repeat expansion of CGG, or the complementary sequence thereof. The hybridization probe can be used for a southern blotting, for example. The southern blotting can detect the repeat expansion. The hybridization probe is configured to detect fragmented nucleic acids that contain the expanded repeat sequence. The fragmented nucleic acids are prepared by using a restriction enzyme. The restriction enzyme is appropriately selected. A restriction site neighboring the expanded repeat sequence is preferably selected. The size of the fragmented nucleic acids prepared by the restriction enzyme may be less than 20 kb, less than 10 kb, or less than 5 kb.

The hybridization probe may comprise a complementary sequence of CGG, or a complementary sequence thereof. The hybridization probe may comprise a complementary sequence of a genome sequence around the expanded repeat sequence. The hybridization probe may comprise a complementary sequence of a sequence flanking the repeat expansion of CGG, or a complementary sequence thereof. The size of the sequence flanking the repeat expansion of CGG may be below 20 kb, below 10 kb, or below 5 kb. The hybridization probe may comprise a complementary sequence of a genome sequence of a partial sequence of the fragmented nucleic acids that contain the expanded repeat sequence.

### Example 1: Identification of CGG Repeat Expansions in Patients with NIID

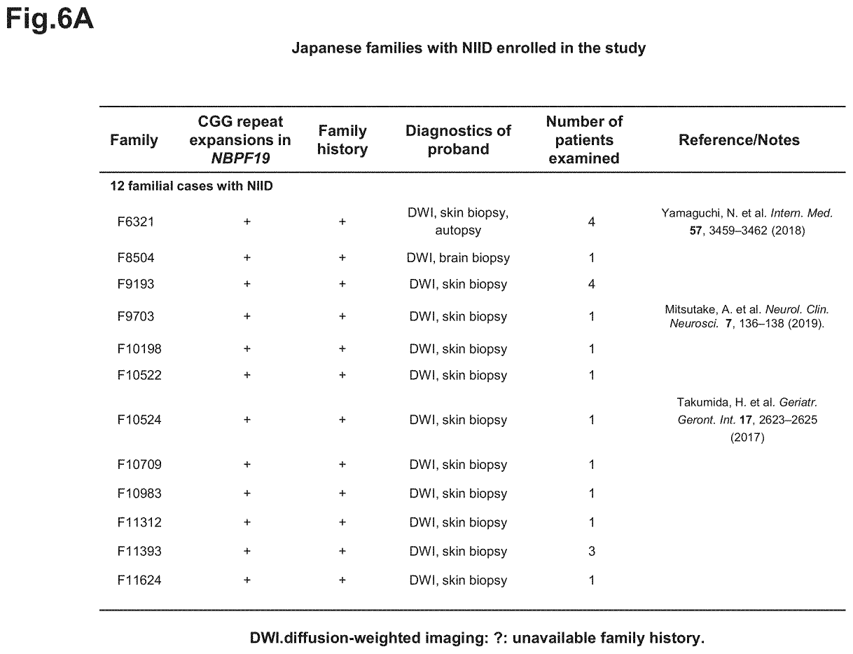

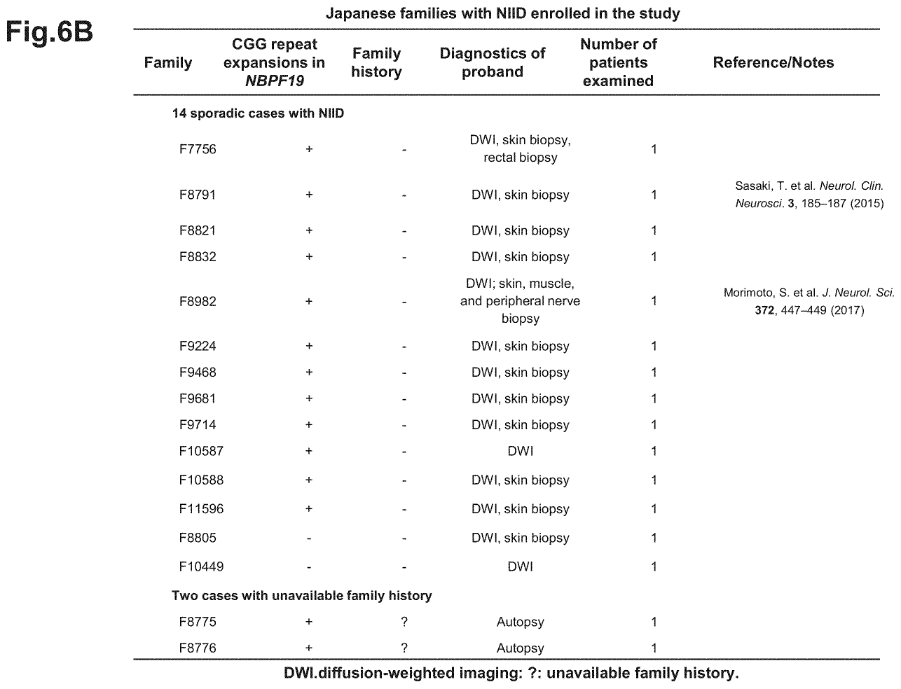

The present inventors first enrolled 12 families with neuronal intranuclear inclusion disease (MID), 14 patients with sporadic NIID, and 2 patients with unavailable family history of NIID, for whom the diagnosis was made on the basis of characteristic MRI findings (MCP sign and high-intensity signals on diffusion-weighted imaging (DWI) in the corticomedullary junction, FIG. 1) and/or intranuclear inclusions in skin or brain tissues (FIG. 6).

The strategy for identification of expanded repeat expansions in the short reads obtained by massively parallel sequencers is shown in FIG. 2. Using TRhist, which extracts short reads filled with tandem repeats and provides histograms classified on the basis of the repeat motifs, short reads overrepresented exclusively in the patients are identified (Step 1). The location of the short reads filled with tandem repeats is determined by alignment of the paired short reads that do not contain repeat motifs (nonrepeat reads) to the reference human genome sequence (Step 2). The expanded repeat sequences are confirmed by repeat-primed PCR analysis, Southern blot analysis, or long-read sequence analysis (Step 3).

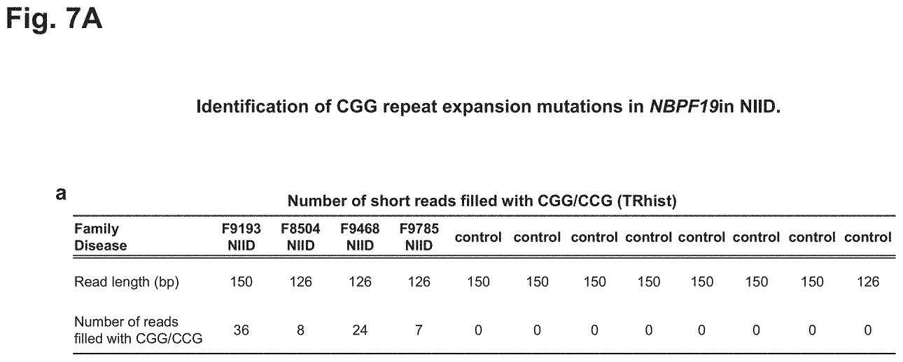

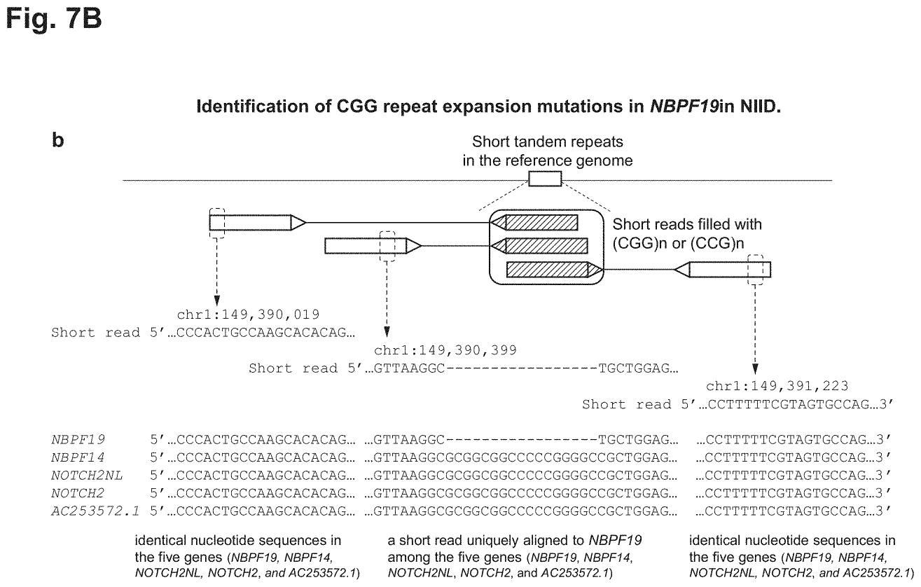

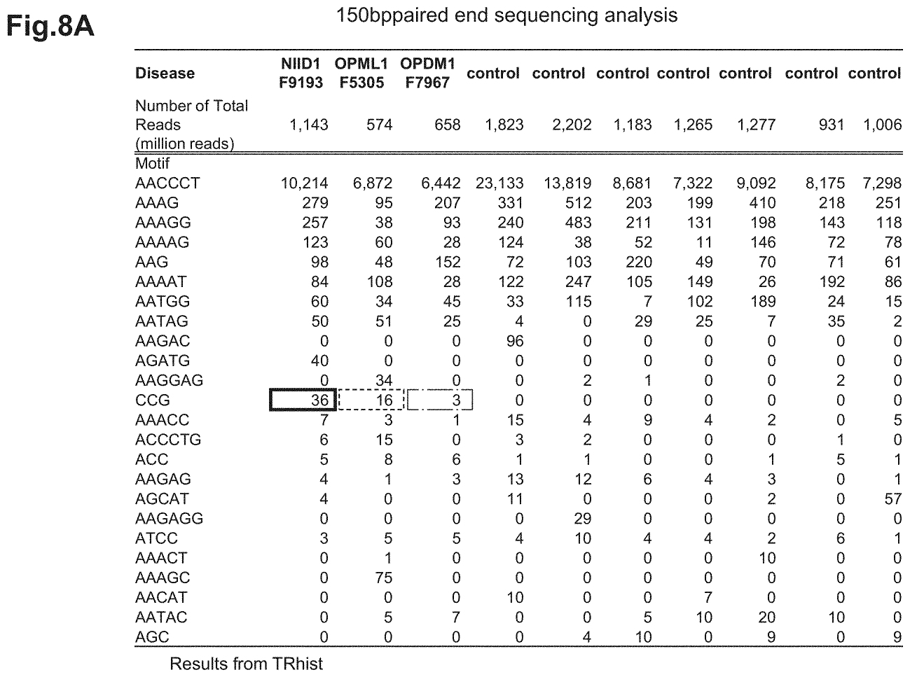

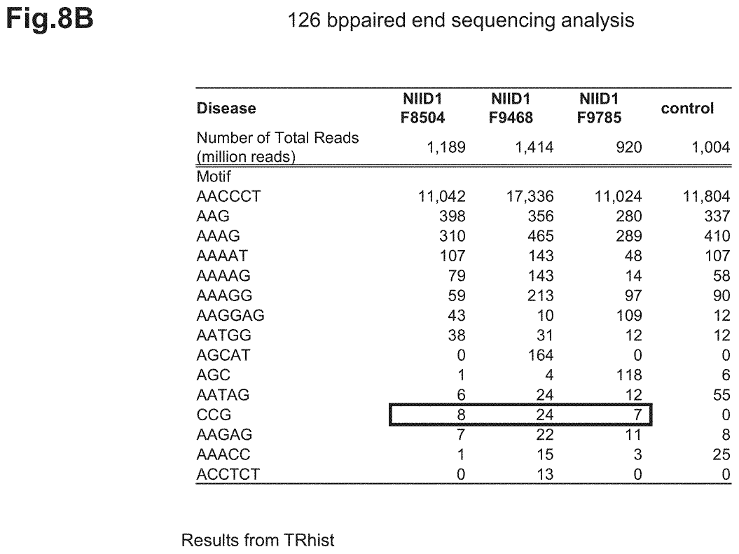

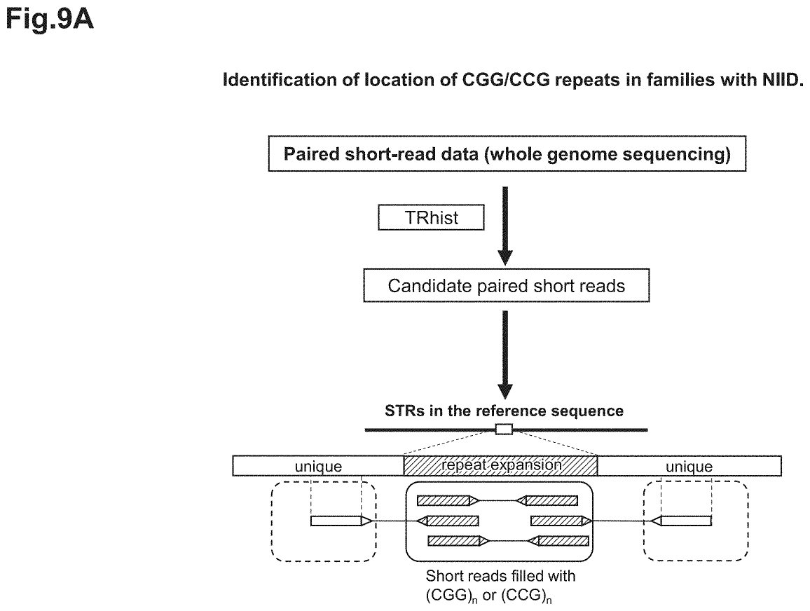

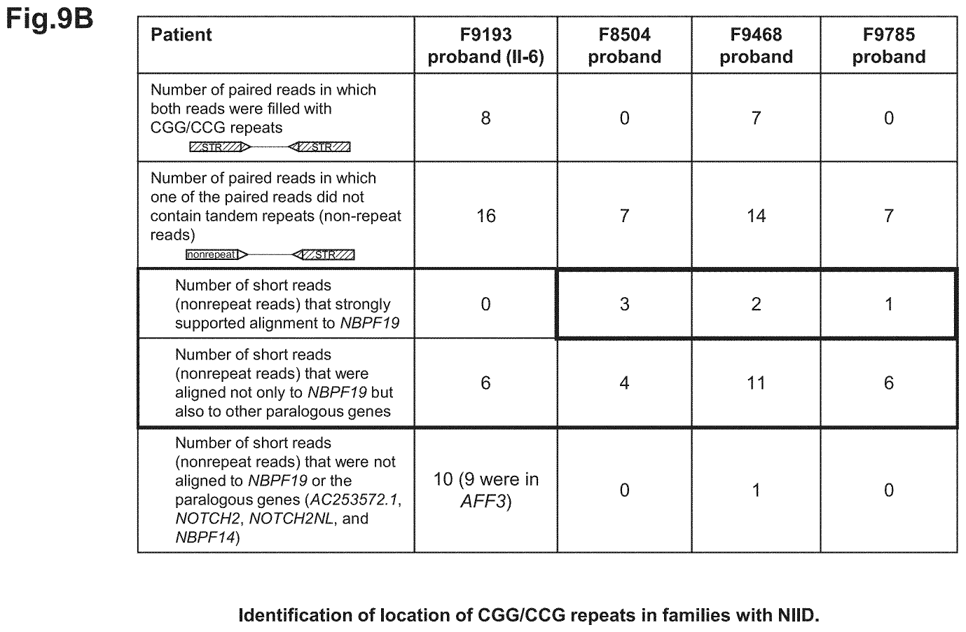

Initially, the present inventors directly searched for paired-end short reads in the whole-genome sequence data of four affected individuals from families F9193, F8504, F9468, and F9785 using TRhist. The present inventors detected short reads filled with CGG repeats that were exclusively observed in the four patients (FIG. 7 and FIG. 8). The alignment of the nonrepeat reads paired with short reads filled with CGG/CCG repeats to the reference genome (hg38) revealed that the CGG repeat expansion was located in the peri-centromeric region of chromosome 1 (FIG. 7). There are five paralogs that have sequences with enormously high identities (>99%) in hg38 derived from the human-, Denisovan-, and Neanderthal-specific multiplication of NBPF gene families in chromosome 1, namely, AC253572.1, NOTCH2, NOTCH2NL, NBPF14, and NBPF19 (FIG. 5). Despite the enormously high identities among these paralogous genes, with careful inspections of the reads, the present inventors identified six nonrepeat reads from three patients strongly supporting the location of the CGG repeats in the 5′ UTR of NBPF19 (ENST00000621744.4 encoding neuroblastoma breakpoint family, member 19), which has also been recently annotated as NOTCH2NLC (NM_001364013.1 or NM_001364012.1 encoding notch homolog 2 N-terminal-like protein C, FIG. 7 and FIG. 9).

### Example 2: Long-Read Sequencing Determined the Position of CGG Repeat Expansions Located in NBPF19

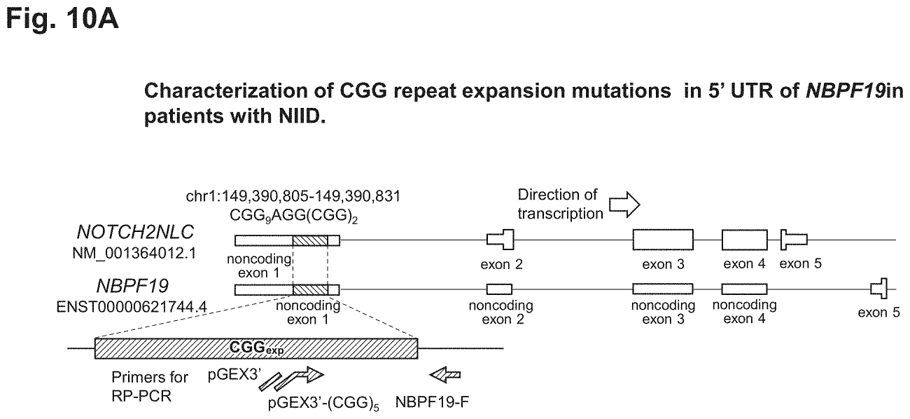

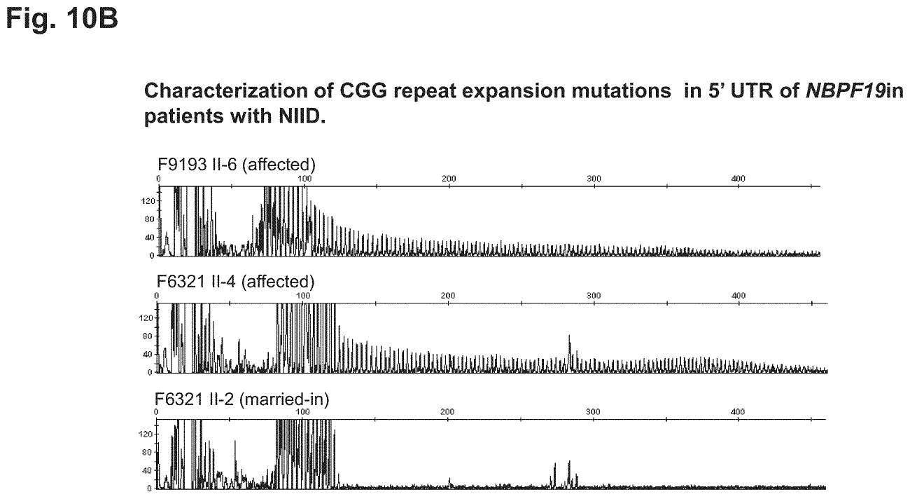

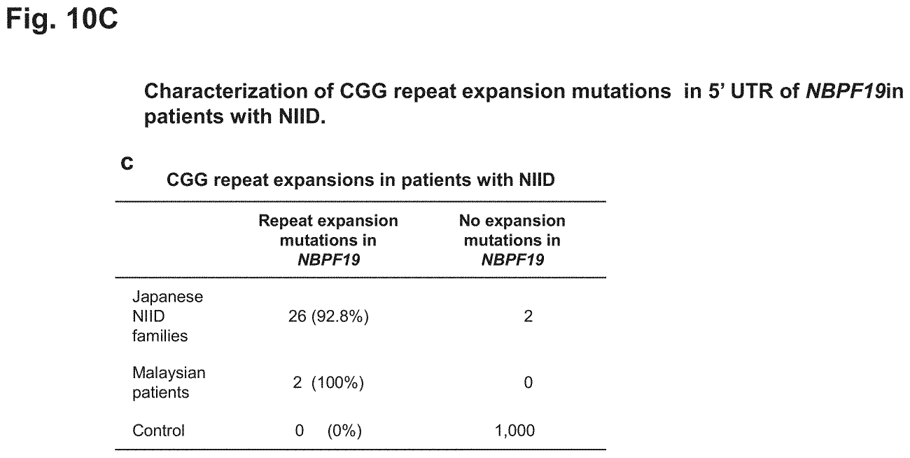

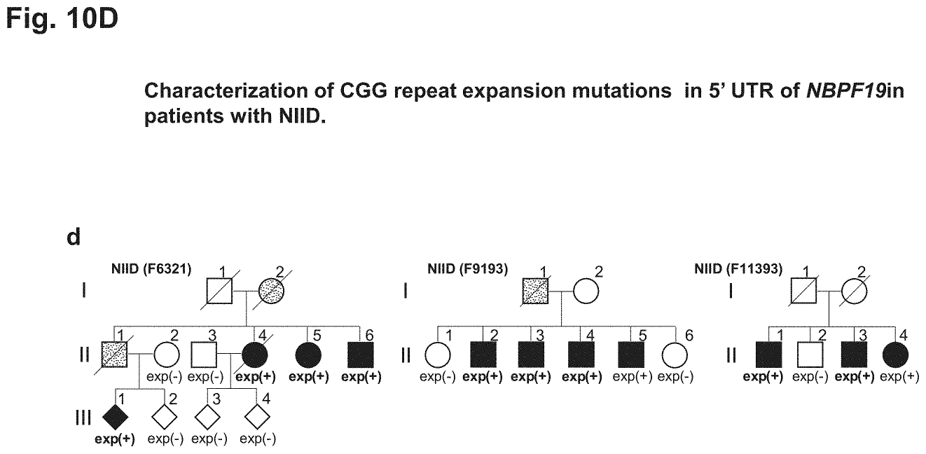

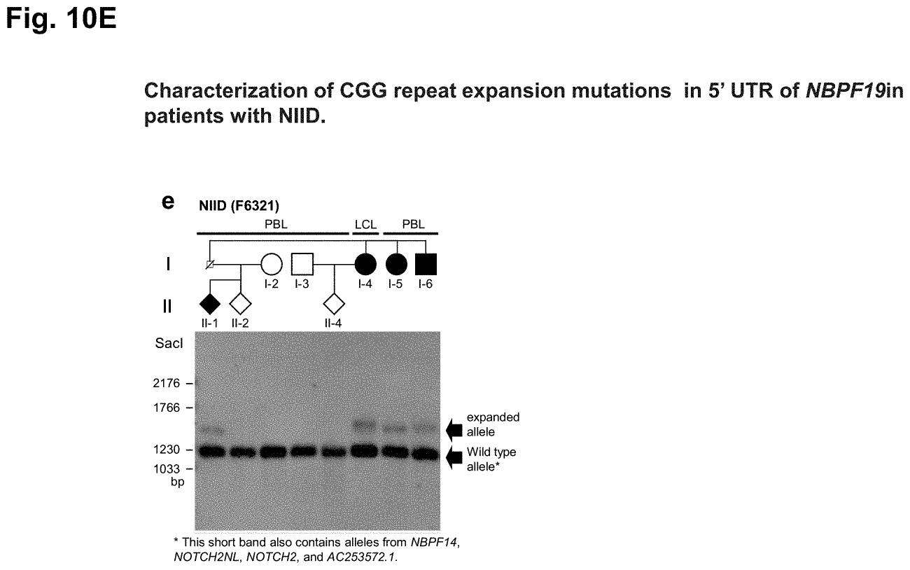

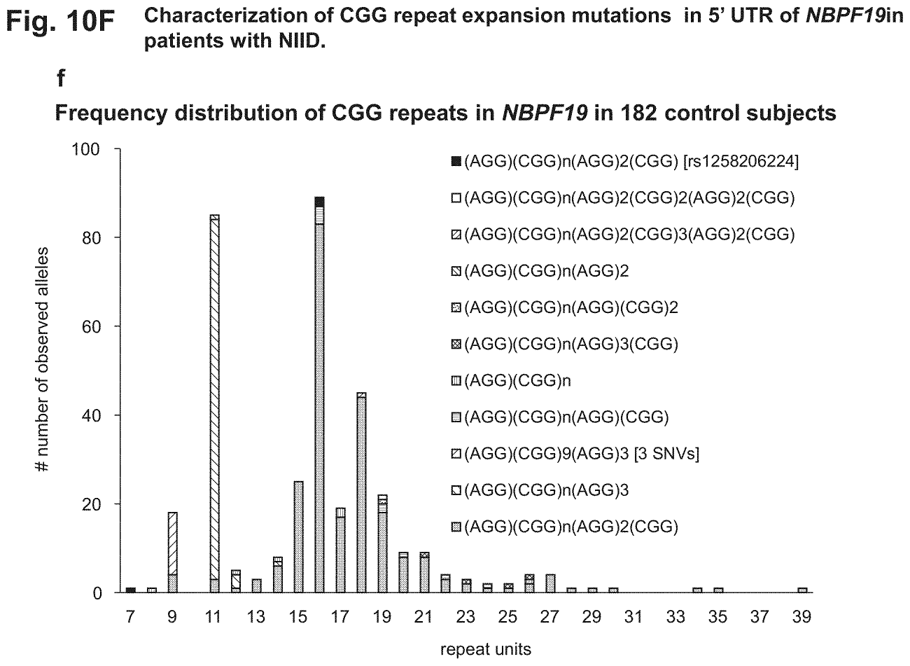

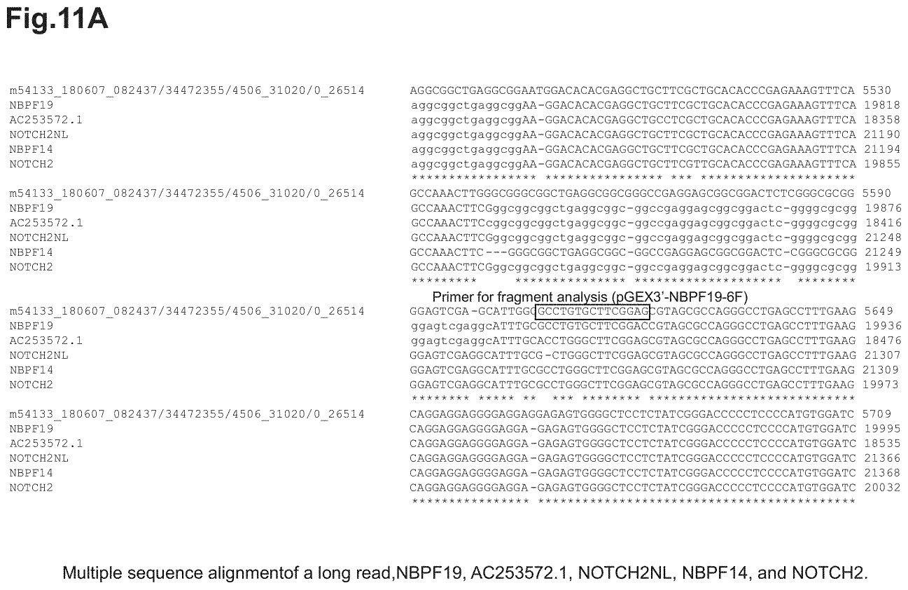

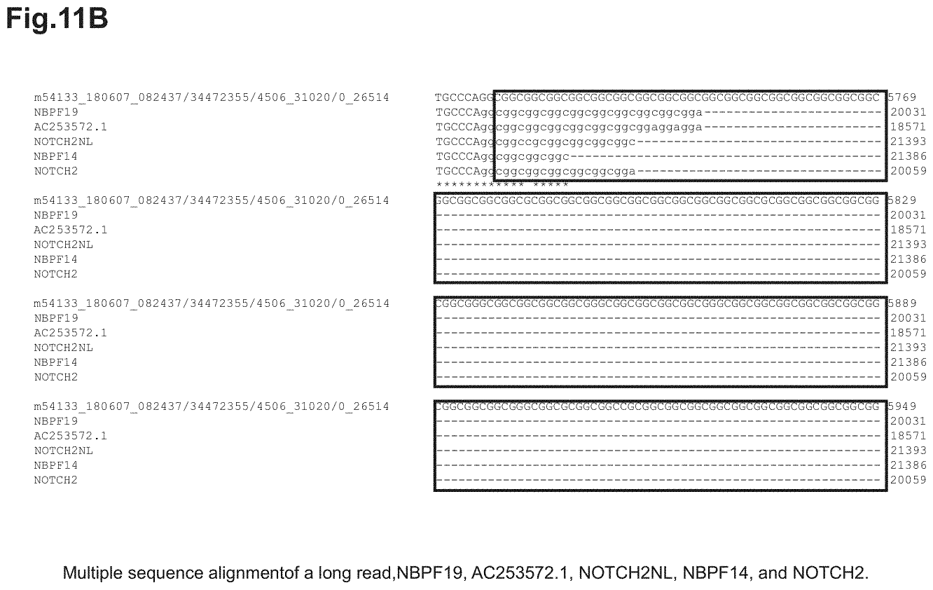

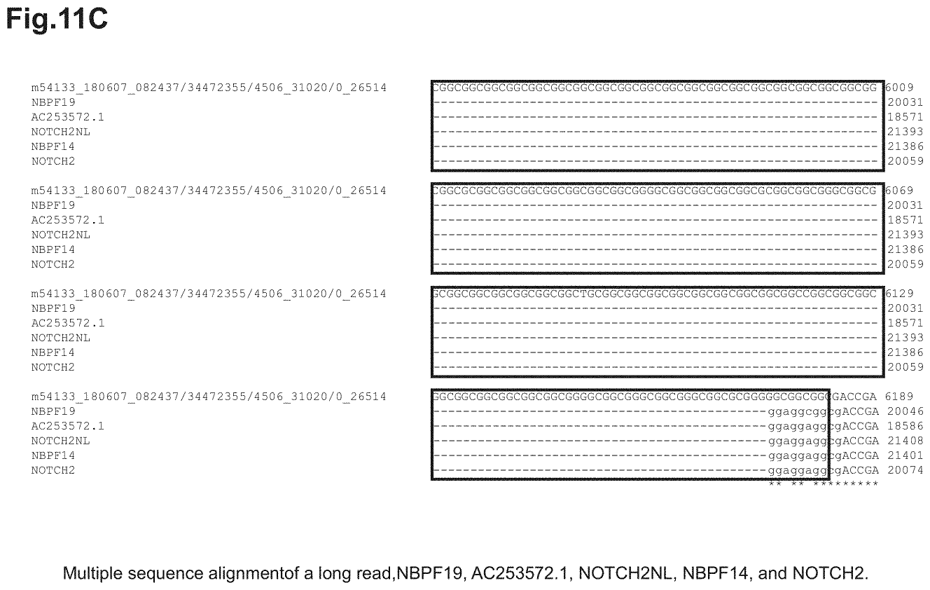

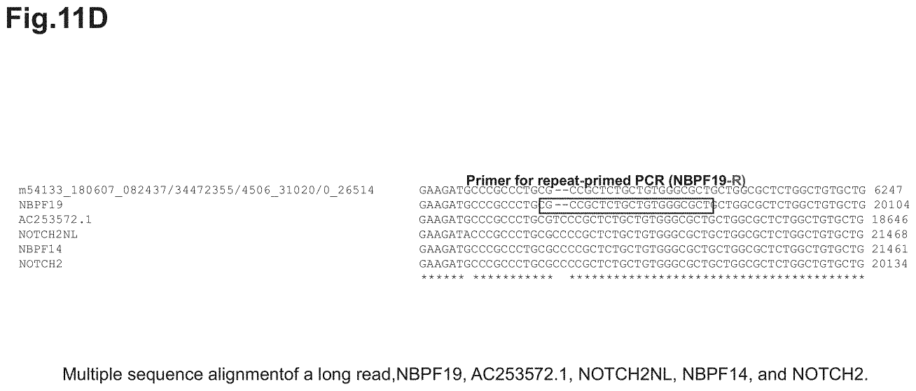

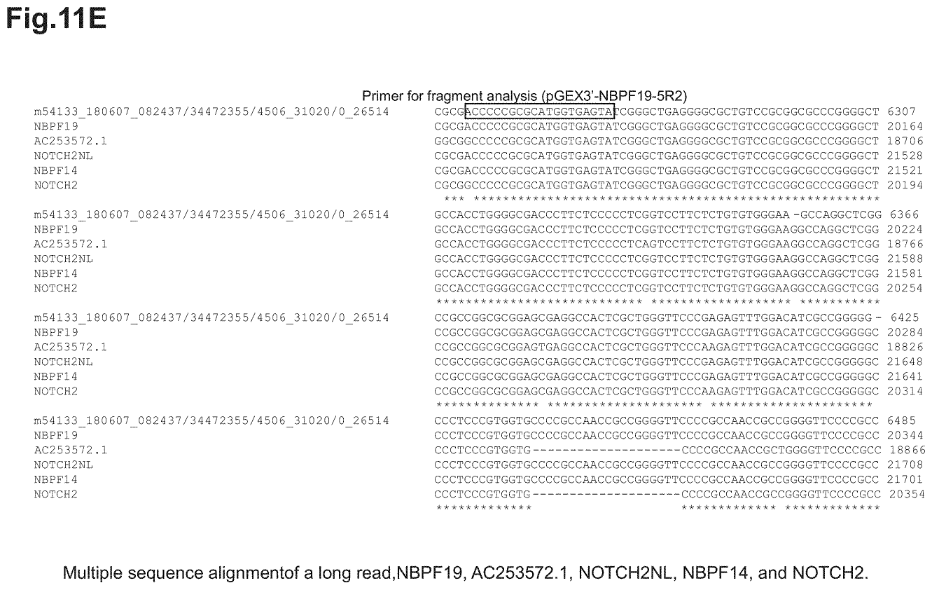

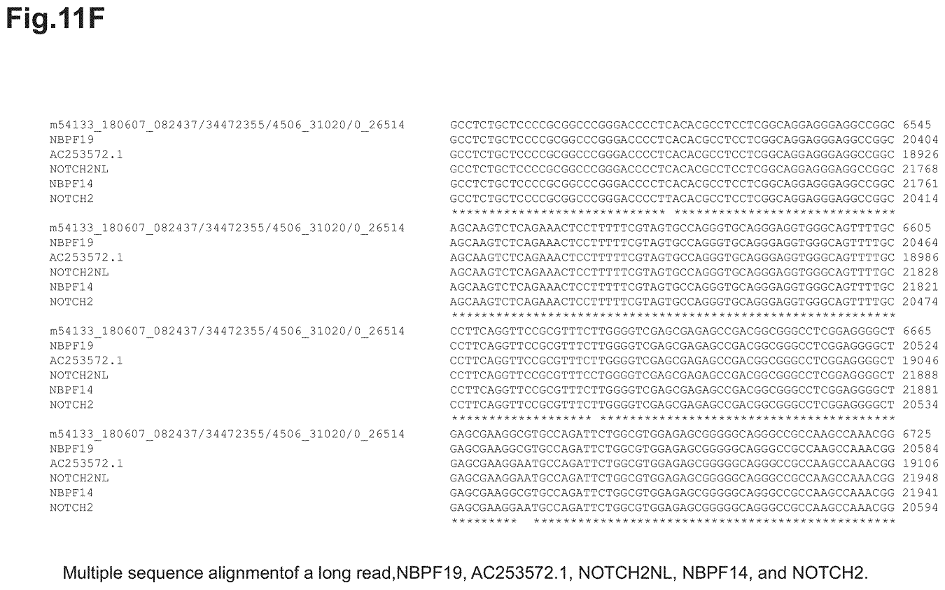

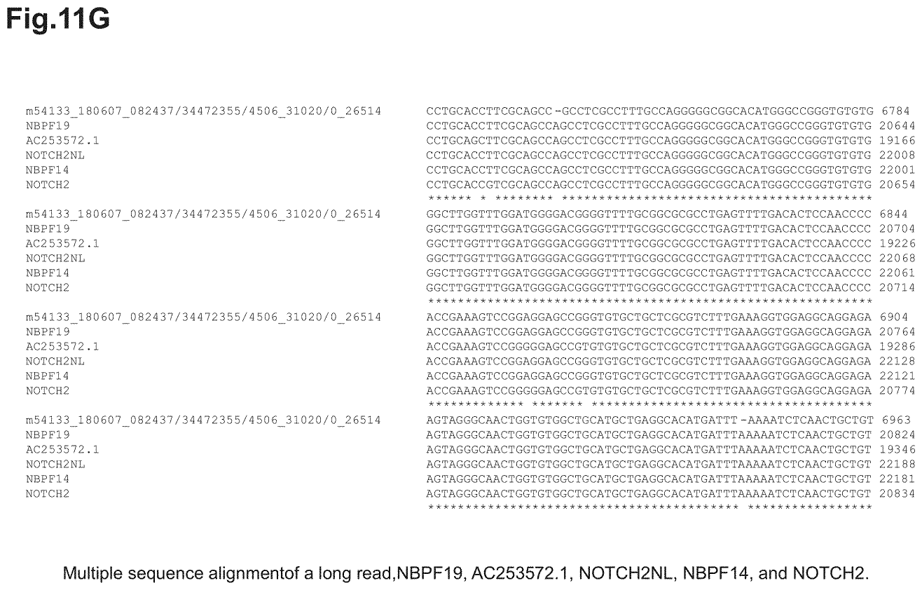

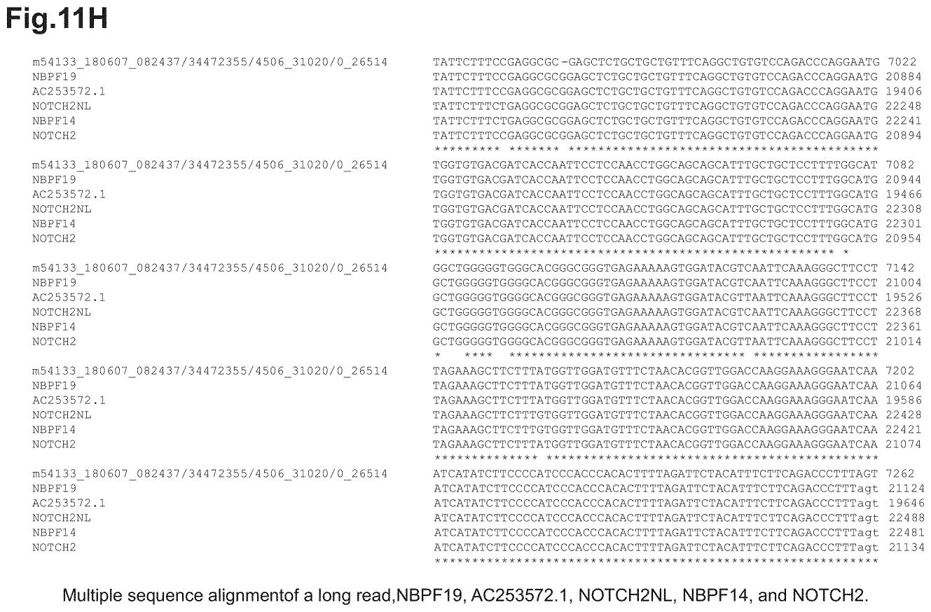

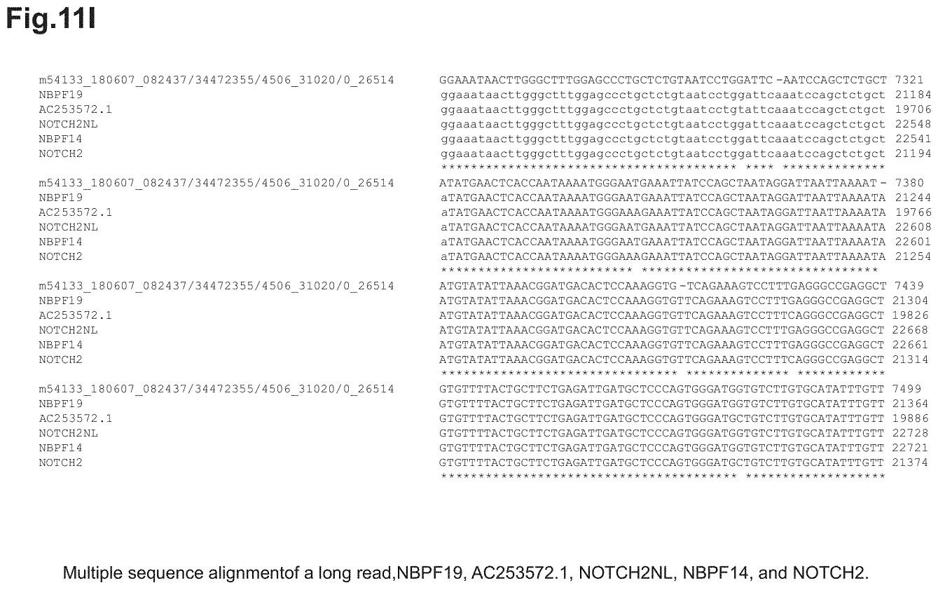

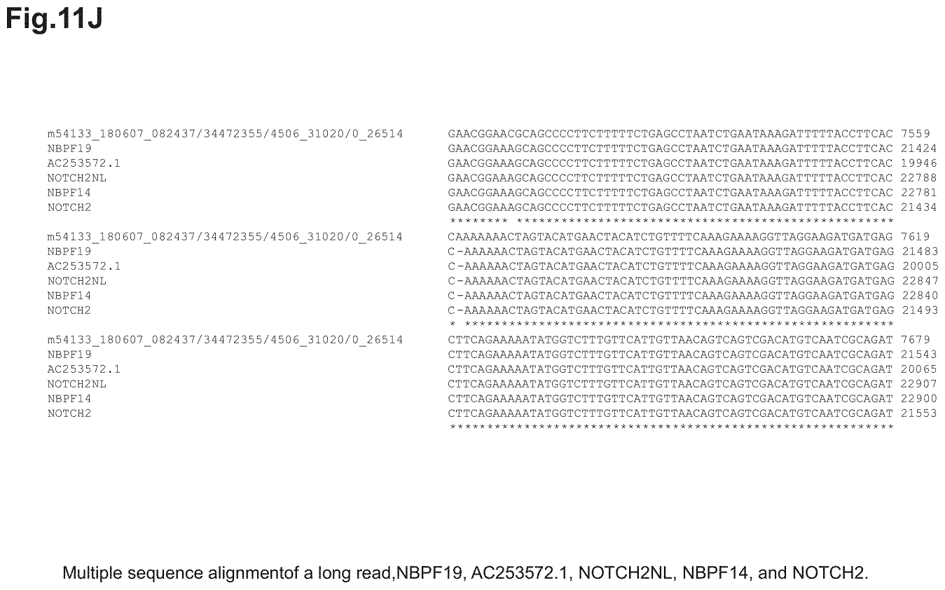

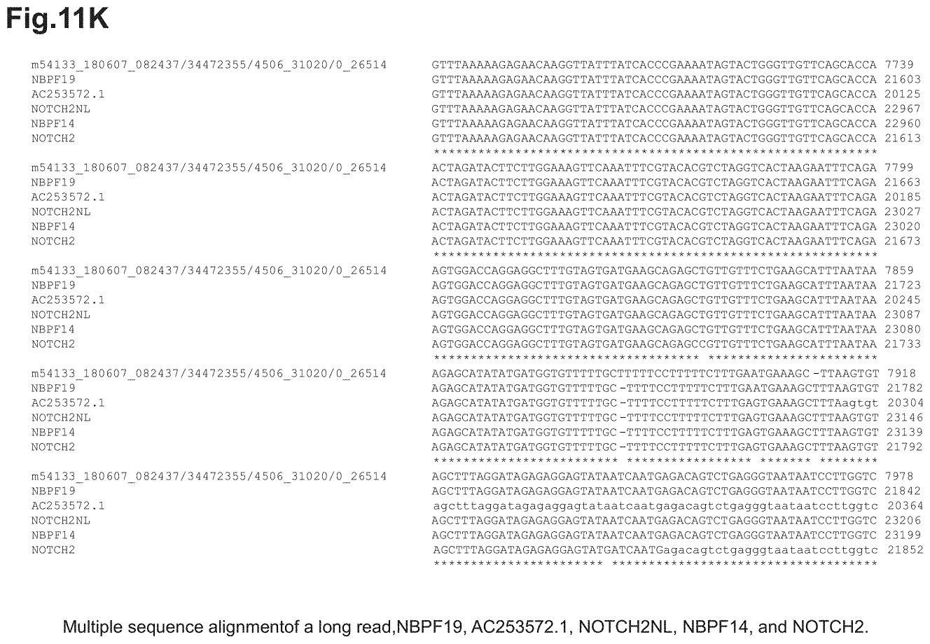

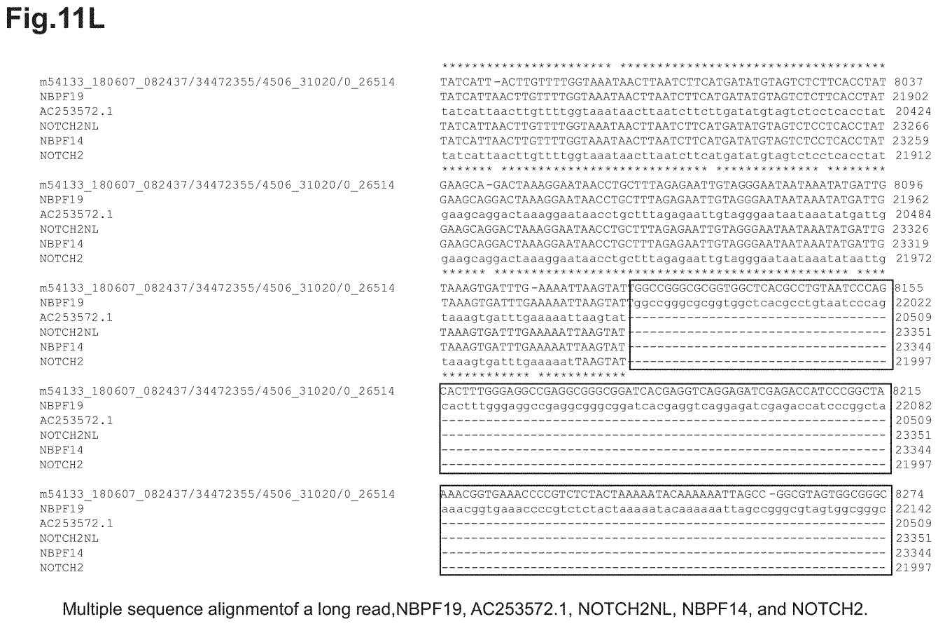

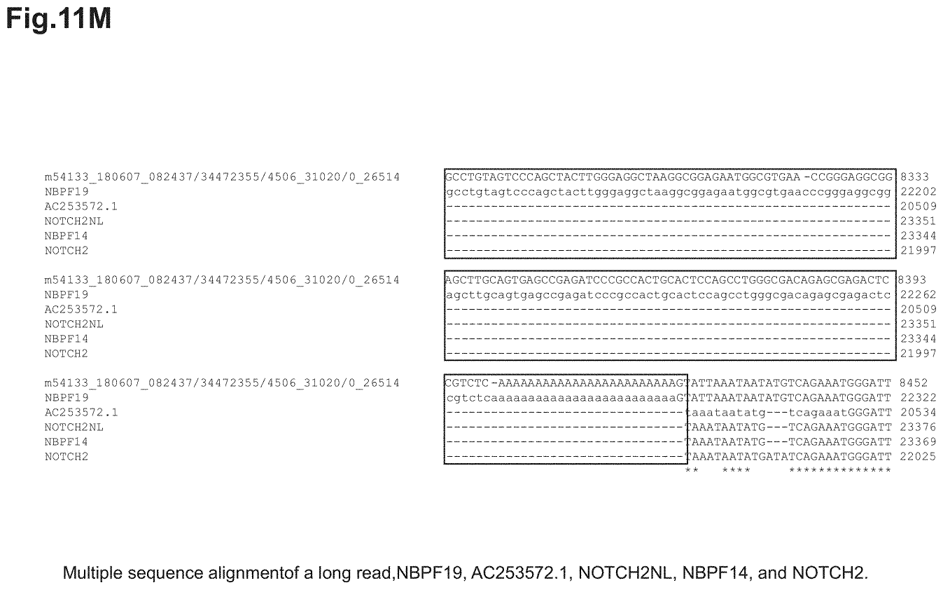

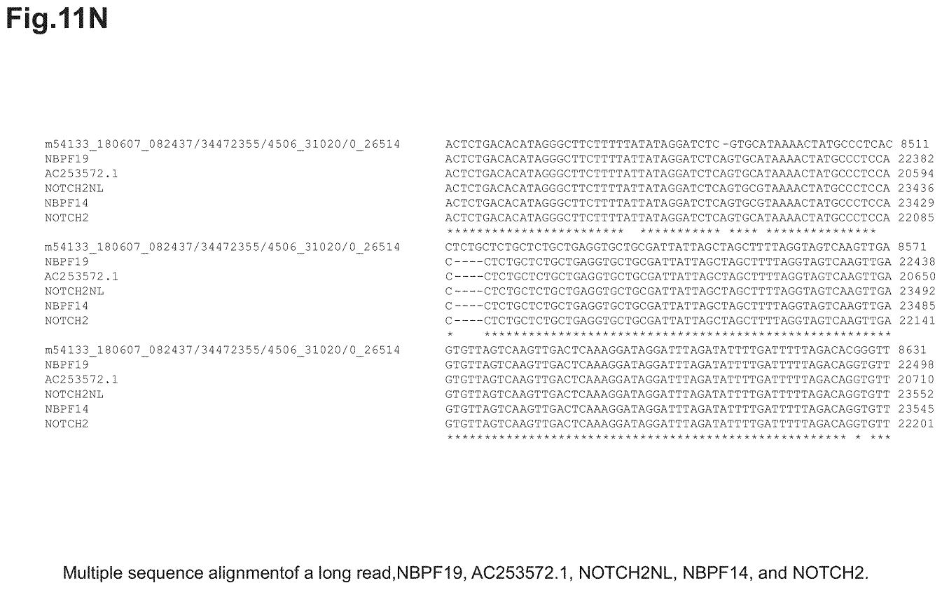

To conclusively determine the position of the repeat expansions, the present inventors conducted single-molecule, real-time (SMRT) sequencing of genomic DNA of patient II-5 in family F9193 (FIG. 10). The present inventors obtained 2,053,214 SMRT subreads with a mean subread length of 6,842 bp. The present inventors aligned these subreads to hg38 using minimap2, and then searched for those originating from the NBPF19 region. Even in the presence of highly identical sequences, the alignment of the subreads containing expanded CGG repeats to NBPF19 (FIG. 10) was clearly supported by the NBPF19-specific insertion of an Alu sequence (FIG. 11).

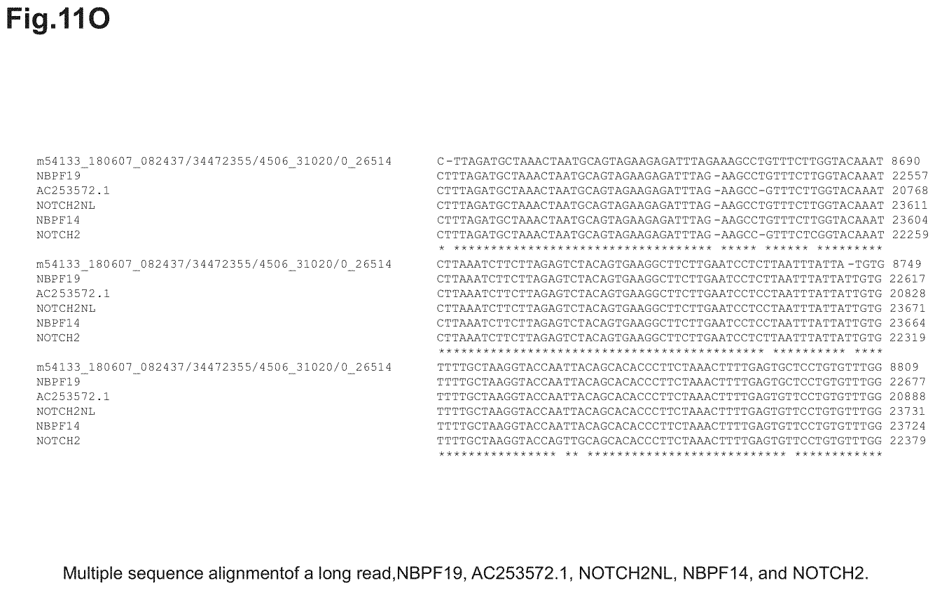

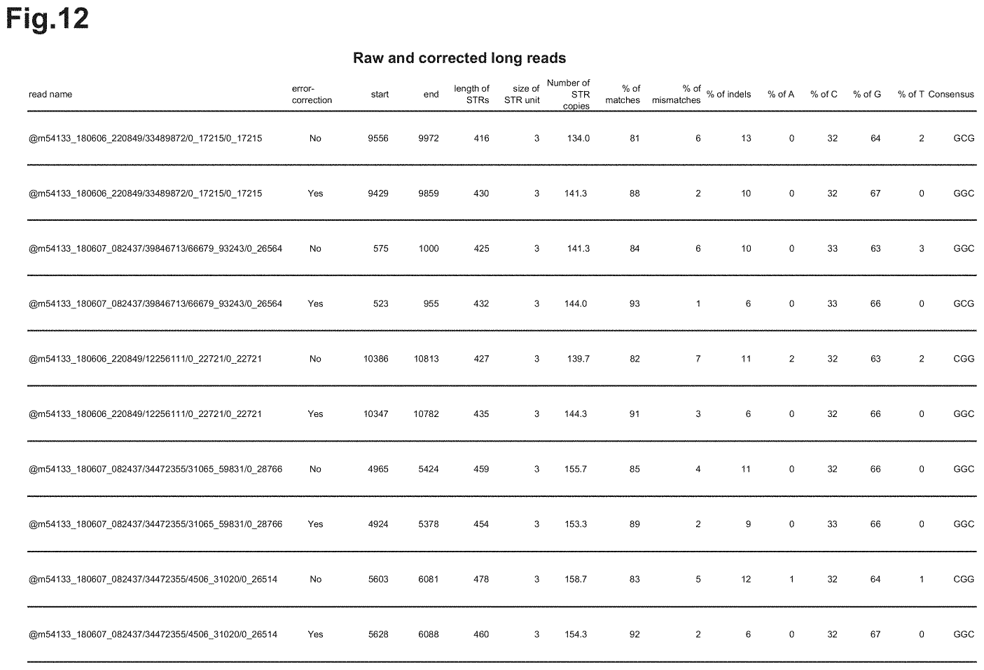

Error correction of the five subreads was made using Canu (version 1.7). Although the error correction improved estimation of the sizes of expanded CGG repeats compared to those of raw subreads (FIG. 12), the five expanded CGG repeats in the error-corrected subreads were slightly different in length; namely, 430, 432, 435, 454, and 460 bp, which may reflect a slight divergence of expanded CGG repeats in somatic cells or may be introduced by the long-read sequencing errors.

### Example 3: Repeat-Primed PCR Analysis and Southern Blot Analysis of Repeat Expansions in NBPF19

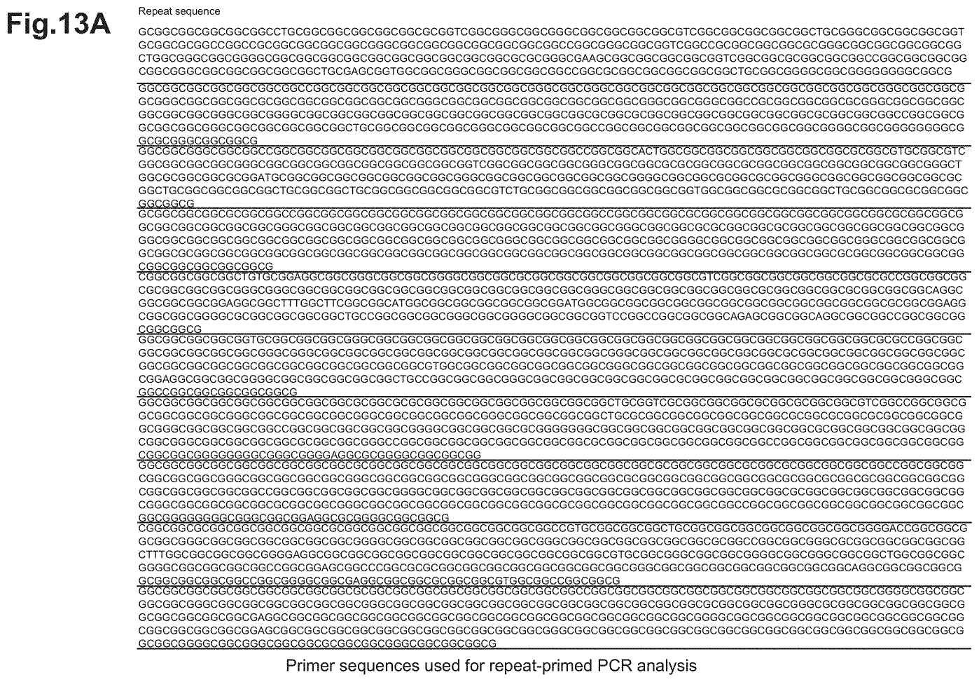

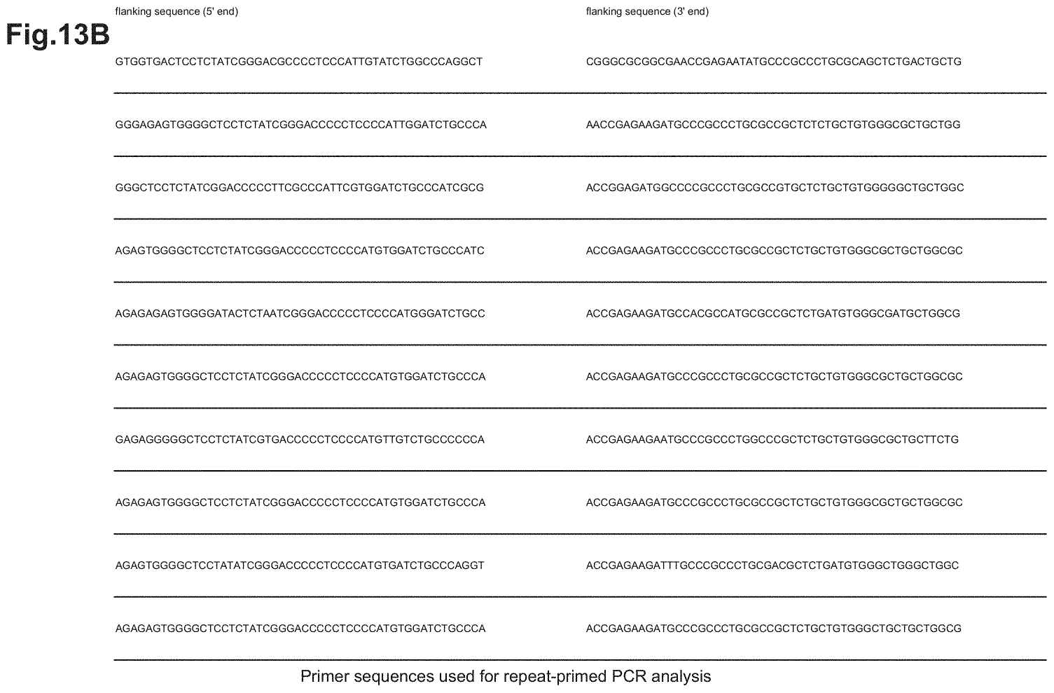

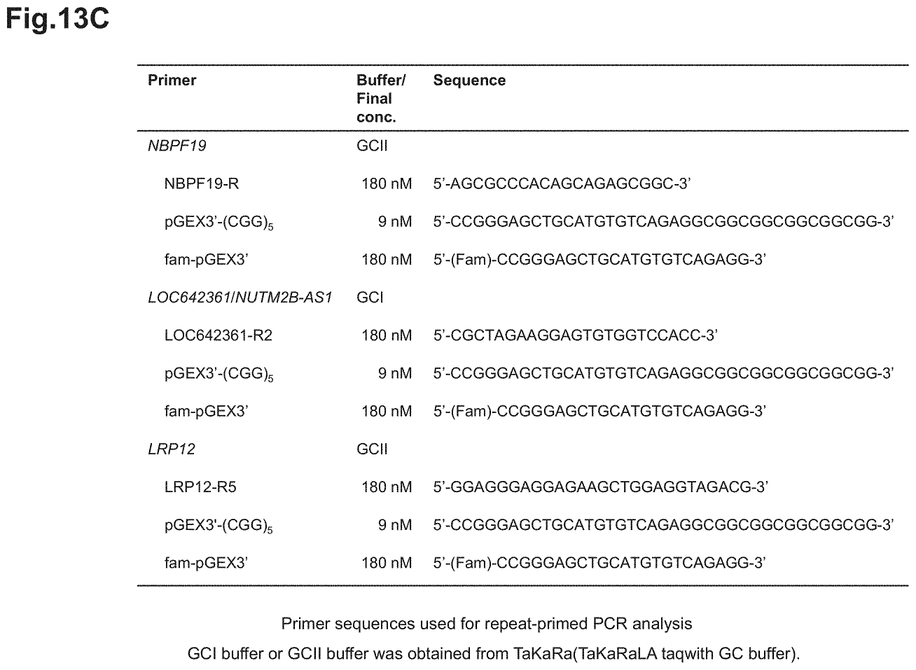

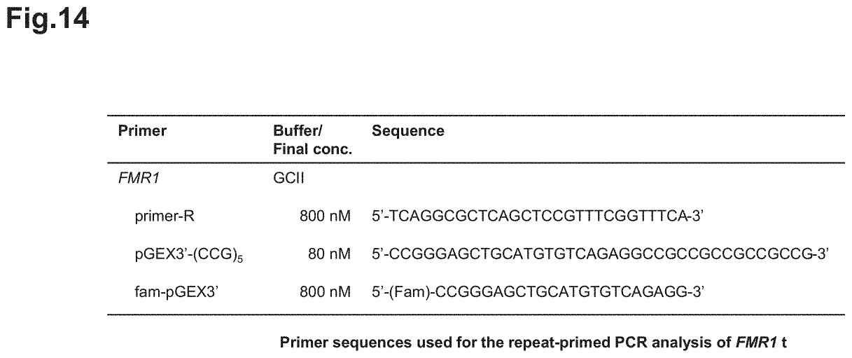

The present inventors then designed the primer set for repeat-primed PCR analysis targeting the expanded CGG repeats in the 5′ UTR of NBPF19 (FIG. 10) based on the NBPF19-specific sequence (FIG. 11 and FIG. 13). The repeat-primed PCR analysis (FIG. 10) indeed demonstrated repeat expansion mutations in 26 of the 28 Japanese index patients with NIID (12 probands of the 12 NIID families, 12 of the 14 patients with sporadic NIID, and both of the two NIID patients with unavailable family histories, FIG. 10 and FIG. 6). None of the 1,000 Japanese controls showed repeat expansions. In the three families with multiple affected family members, all the 11 affected individuals had the repeat expansions, whereas three asymptomatic individuals with normal nerve conduction study findings in family F6321, three asymptomatic individuals aged >60 years with normal MRI findings in families F9193 and F11393, and two married-in healthy individuals did not (FIG. 10). Additionally, the repeat expansion mutations were also identified in two Malaysian males of Chinese origin. Patient 1 presented with tremor, ataxia, peripheral neuropathy, urinary incontinence, and cognitive decline with the age at onset of 53 years, and patient 2 with unusual resting and action upper limb tremor, gait ataxia, and urinary incontinence with the onset in the middle age). Characteristic MRI findings (MCP sign and T2 hyperintensity signals in the white matter) suggested the diagnosis of FXTAS, but they did not have CGG repeat expansion mutations in FMR1 as examined by repeat-primed PCR analysis (FIG. 14).

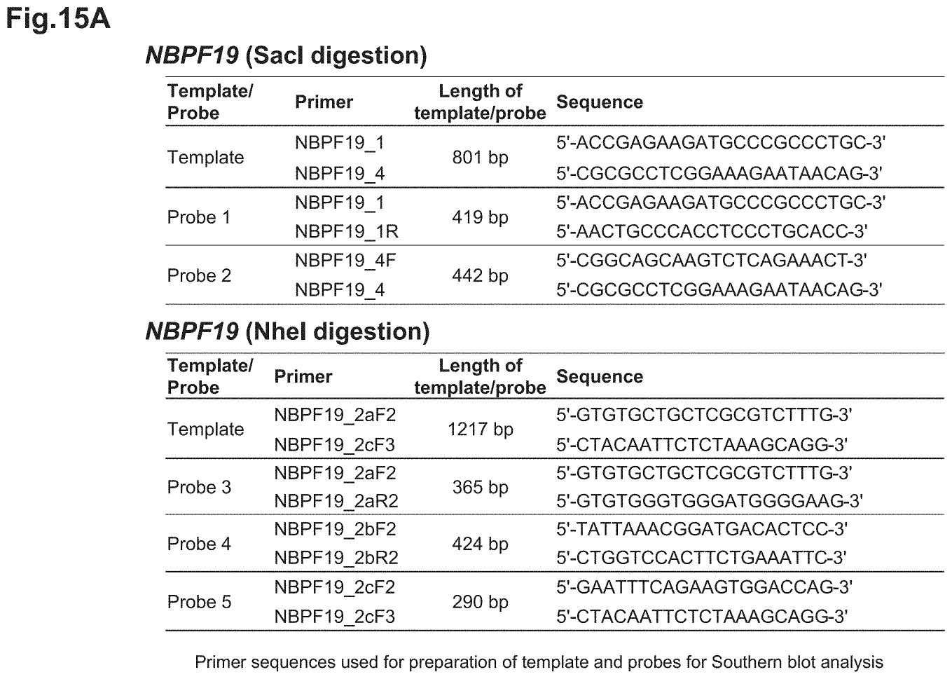

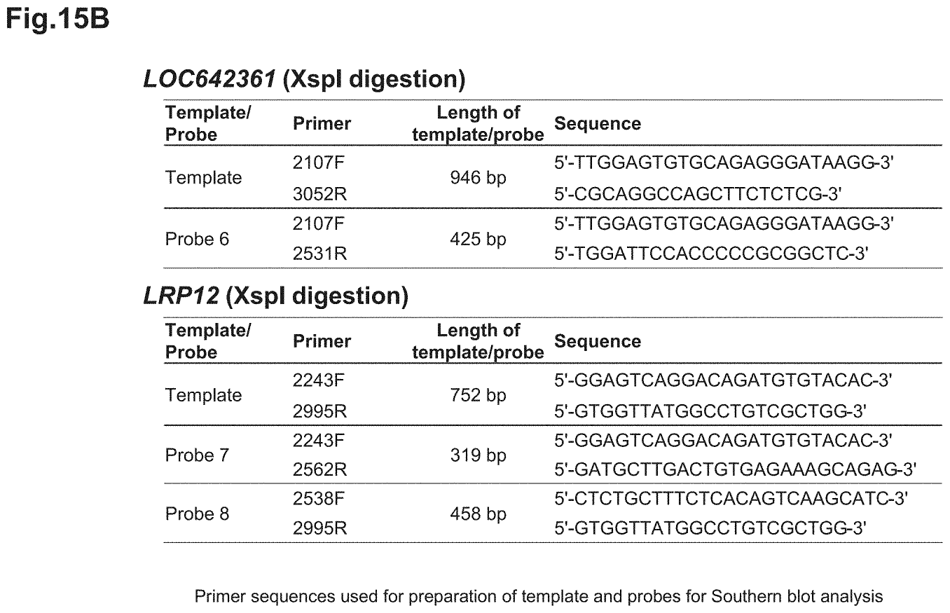

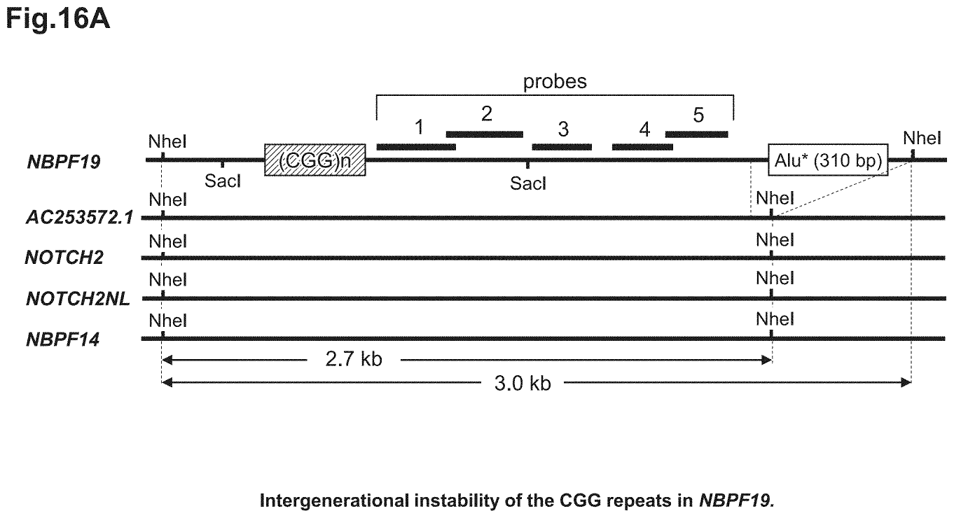

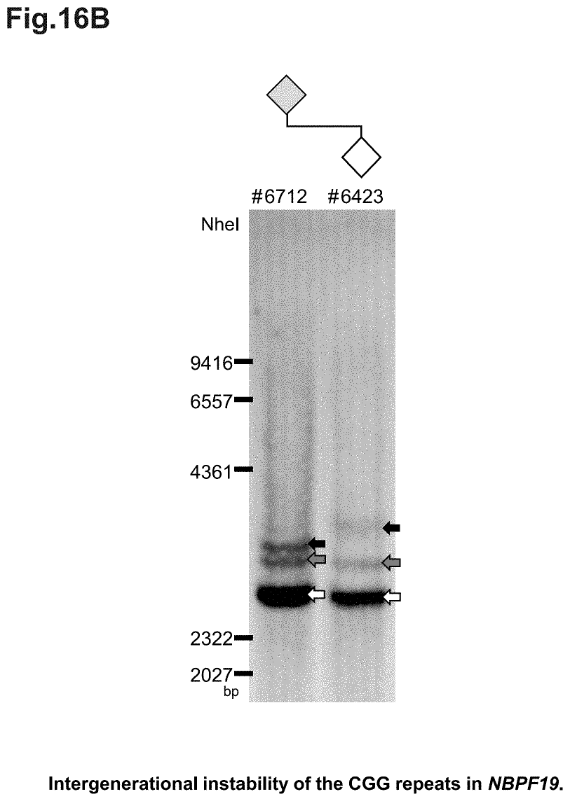

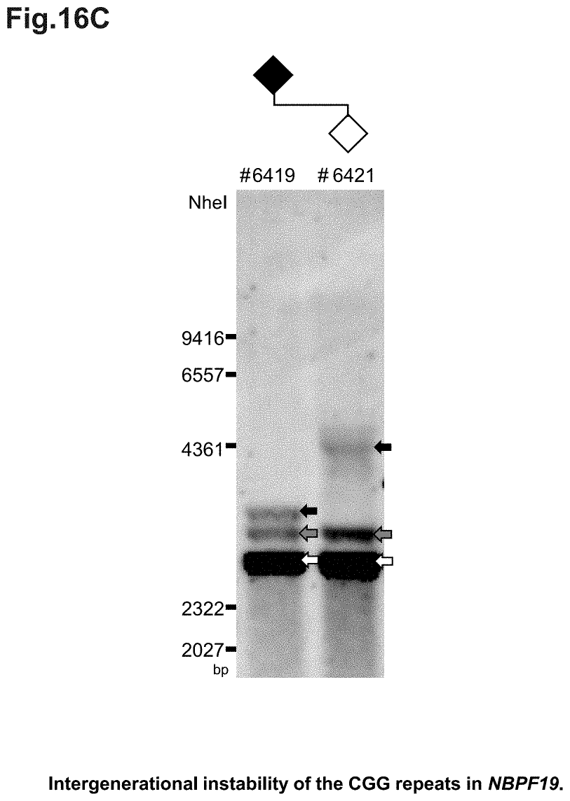

The present inventors further confirmed the CGG repeat expansions in NIID patients by Southern blot analysis. The probes were designed to target the sequences flanking the CGG repeat in NBPF19 (FIG. 15). Although the expanded alleles were clearly shown, strong signals reflecting the wild-type alleles of NBPF19 and fragments of the same sizes derived from the other four paralogous genes were detected owing to the highly identical sequences (FIG. 10 and FIG. 5). Southern blot analysis of 28 patients with NIID and seven unaffected individuals revealed that all the patients had expanded alleles whereas the unaffected individuals did not. The lengths of the CGG repeat expansion were estimated to range from 270 to 550 bp, corresponding to approximately 90-180 repeat units. Intergenerational instability of expanded repeats was observed by Southern blot analysis of the two parent-offspring pairs (FIG. 16). Since the two offsprings were presymptomatic carriers, the present inventors were unable to address the presence of genetic anticipation phenomenon as a result of intergenerational instability of expanded repeats.

### Example 4: Distribution of Number of CGG Repeat Units and Repeat Configurations in Controls

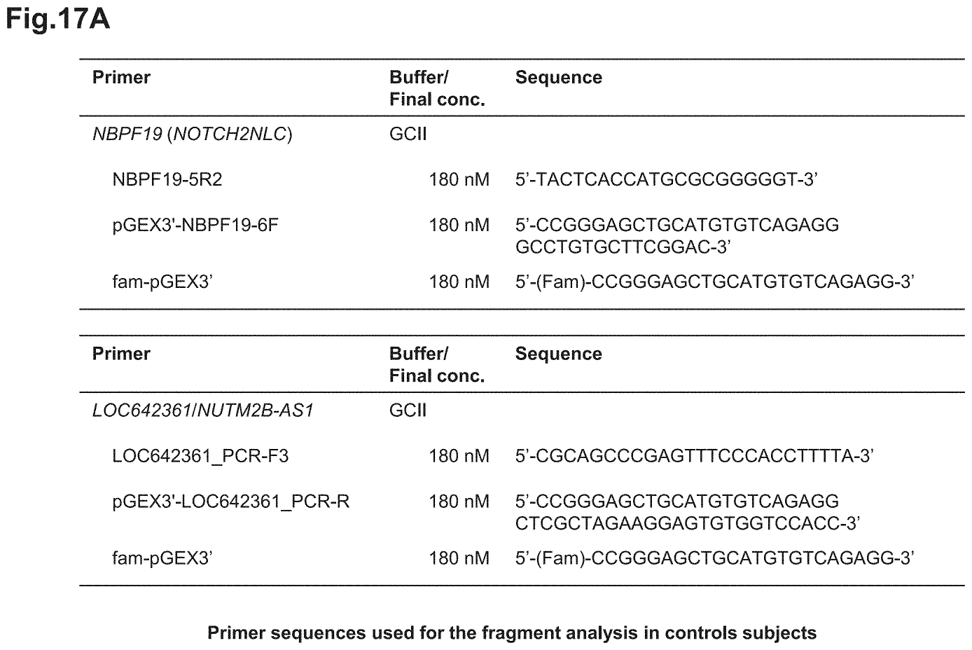

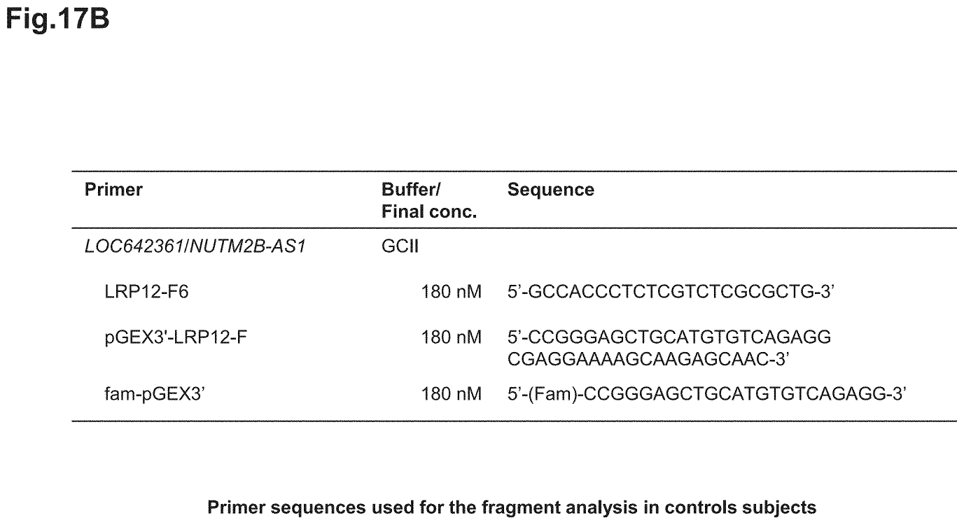

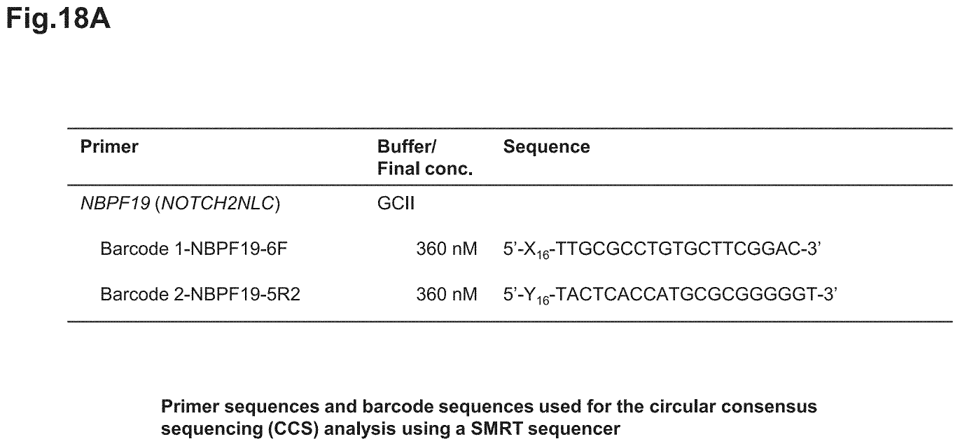

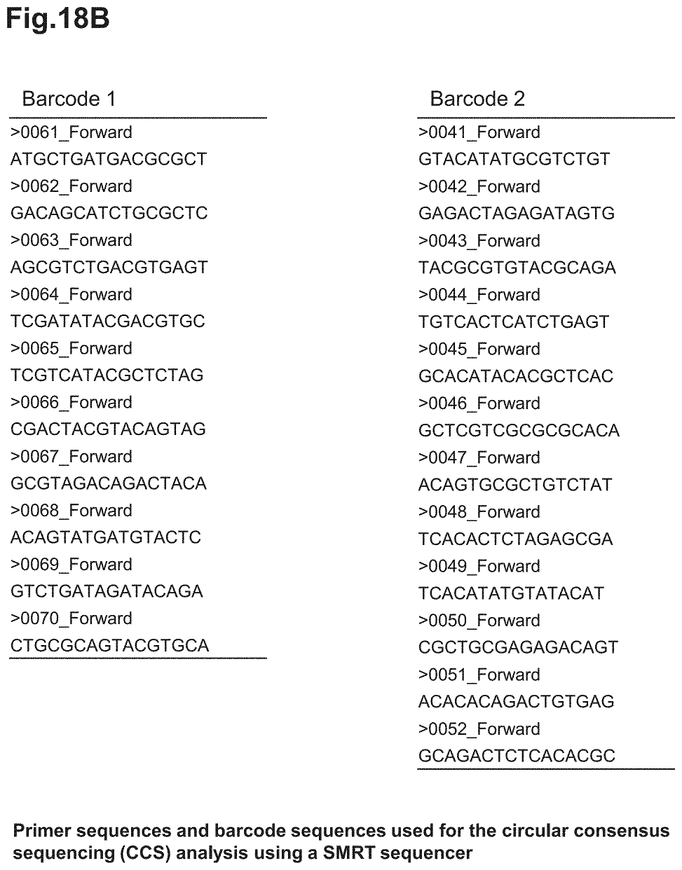

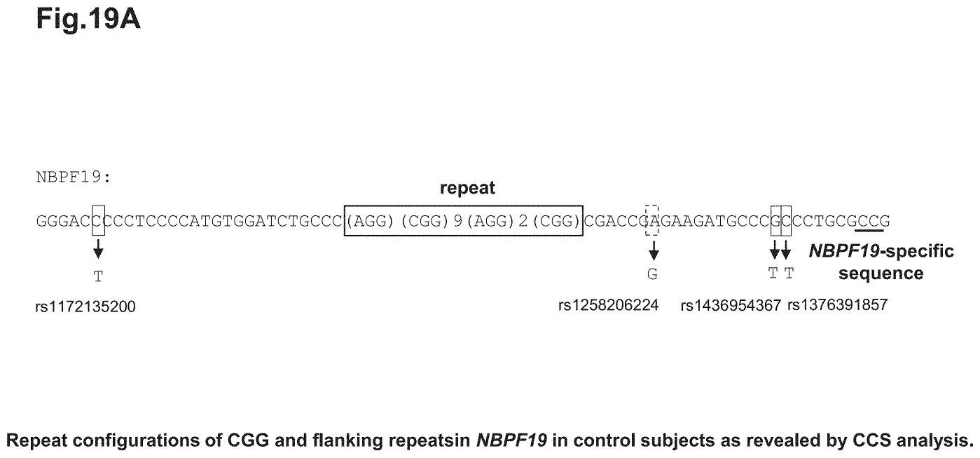

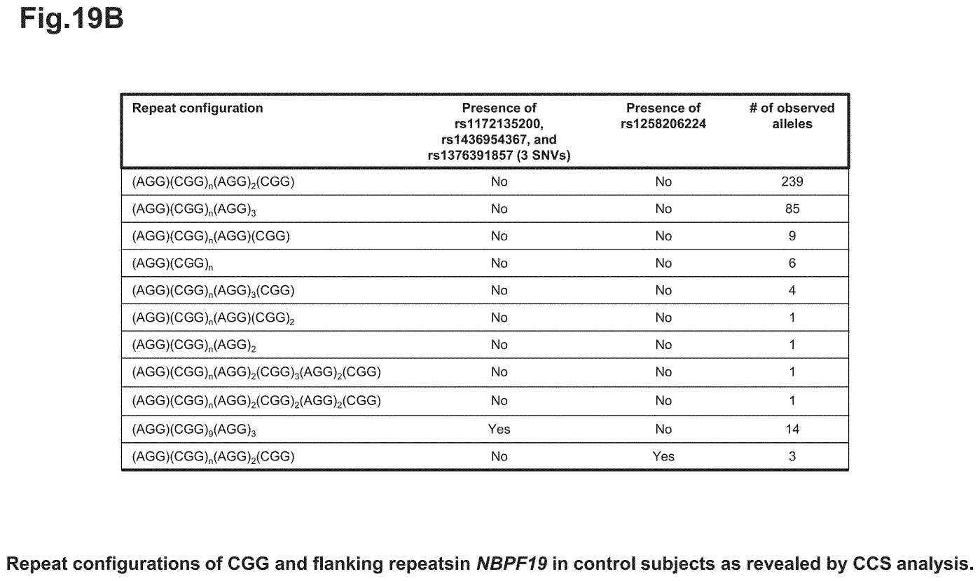

Since the CGG repeats and the flanking sequences of NBPF19 show enormously high identities among the paralogous genes, AC253572.1, NOTCH2, NOTCH2NL, and NBPF14 (FIG. 5, FIG. 7), the present inventors devised an NBPF19-specific primer pair (FIG. 17) to specifically amplify NBPF19 and subjected the PCR products to circular consensus sequencing (CCS) mode of a PacBio Sequel sequencer (Pacific Biosciences) to exactly determine the repeat configurations of CGG repeats in NBPF19 (FIG. 18). CCS analysis of the PCR products revealed polymorphic lengths of the repeat structure as well as 11 repeat configurations (FIG. 10) with the number of CGG repeat units ranging 7-39 in 182 control subjects. Interestingly, one allele carrying three single nucleotide variants (rs1172135200, rs1436954367, and rs1376391857) in the flanking sequences, all of which carried a configuration (AGG)(CGG)₉(AGG)₃, and another allele carrying rs1258206224 with a configuration of (AGG)(CGG)ₙ(AGG)₂(CGG) were observed in 14 and 3 control subjects, respectively (FIG. 19). No single nucleotide variants (SNVs) were observed in other alleles. Reanalysis of long reads spanning the expanded CGG repeats in a patient with NIID revealed a configuration of (AGG)(CGG)ₙ without these SNVs (FIG. 11).

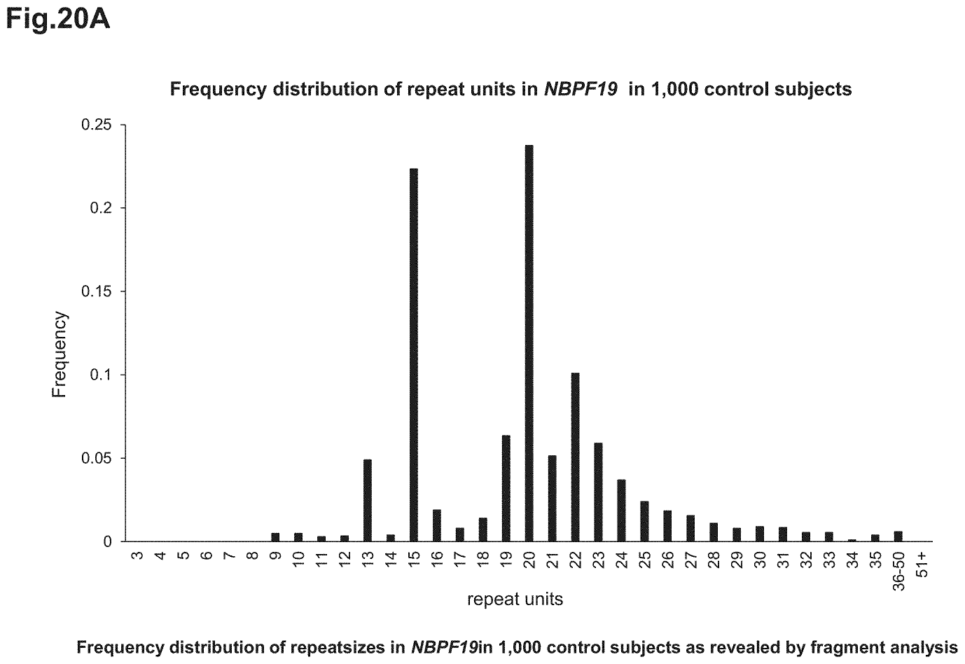

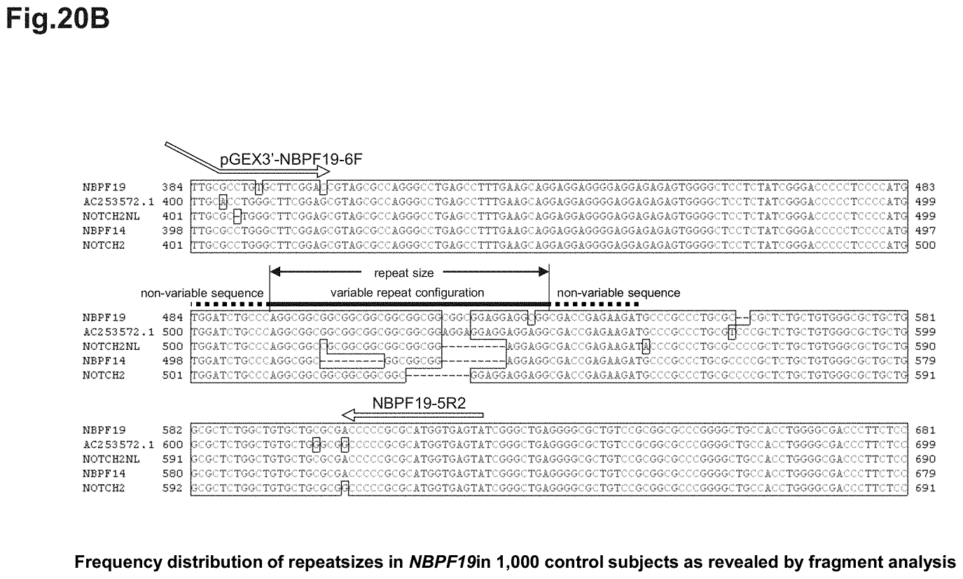

The present inventors furthermore conducted fragment analysis of the PCR products containing the CGG repeats in NBPF19 in 1,000 controls. Since the repeat configurations are variable as shown in FIG. 10, the sizes of the repeats were determined as the sizes of the repeat configurations between the flanking non-variable sequences. The repeat sizes in NBPF19 were 9-43 in 1,000 controls (FIG. 20).

### Example 5: Methylation Status of Expanded CGG Repeats in NBPF19 and Expression Levels of NBPF19 in Brains

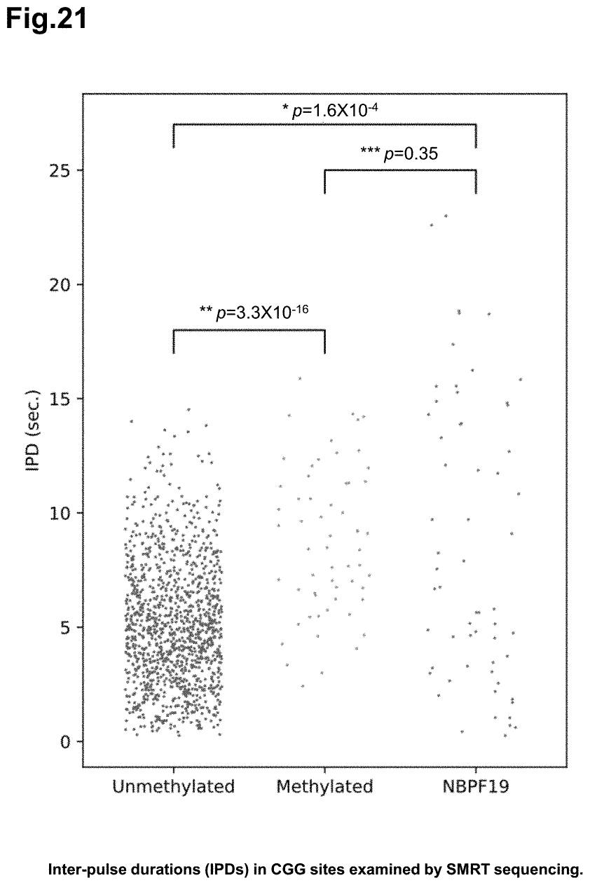

To investigate methylation status of expanded CGG repeats located in the 5′ UTR of NBPF19, the present inventors utilized inter-pulse duration (IPD) analysis of the SMRT sequencing reads obtained from a patient with NIID. Because methylated CpGs slow down the sequencing process and generally result in statistically longer IPDs, the present inventors investigated the distribution of IPDs employing the method the present inventors recently devised. The present inventors found that the IPDs of expanded CGG repeats in the 5′ UTR of NBPF19 was similar to those of hypermethylated CGG repeats as determined by bisulfite sequencing (<30% of bisulfite calls on CpG sites) (p=0.35, n=59, two-sided test) but was significantly dissimilar to those of hypomethylated CGG repeats (>70% of bisulfite calls on CpG sites) (p=1.6*10-4, n=1,220, one-sided test), showing that the expanded CGG repeats in the 5′ UTR of NBPF19 tended to be hypermethylated (FIG. 21).

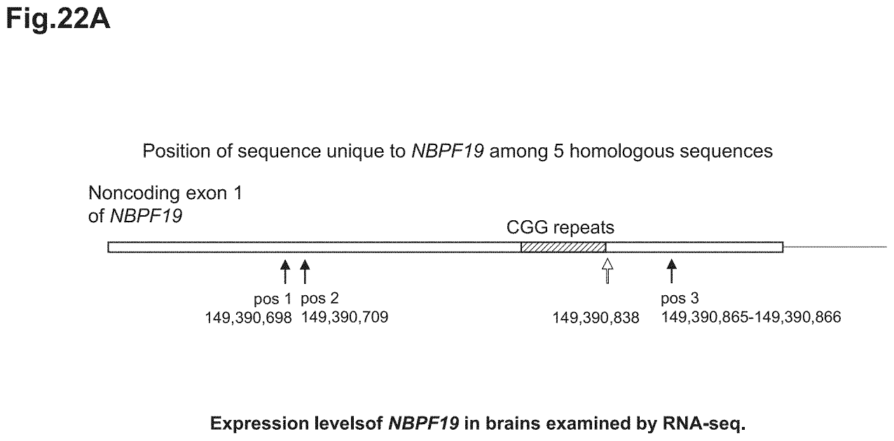

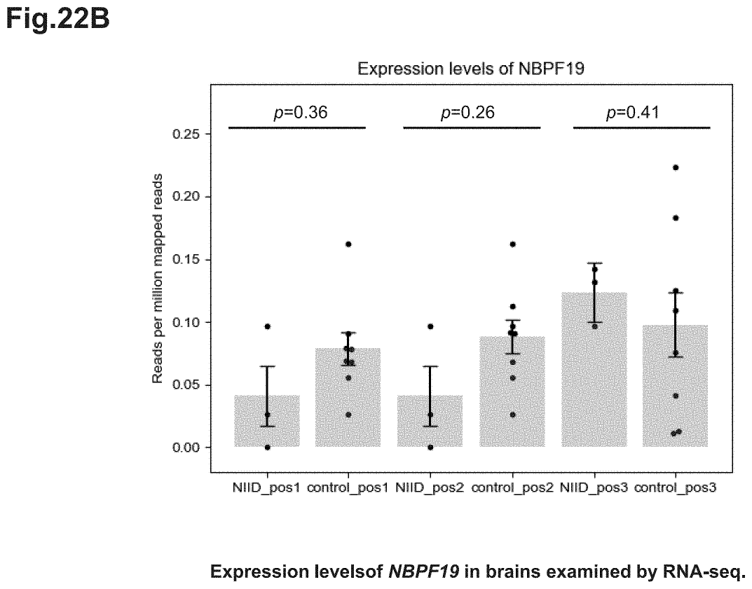

To examine whether the altered methylated status of NBPF19 is associated with transcriptional repression, the present inventors conducted RNA-seq analysis using RNAs extracted from brains of patients with NIID Analysis of the expression levels of transcripts of NBPF19 using NBPF19-specific sequences revealed no statistical difference between expression levels of patients with NIID (n=3) and those of controls (n=8) (FIG. 22).

### Example 6: Identification of CGG Repeat Expansions in LOC642361/NUTM2B-AS1 in OPML

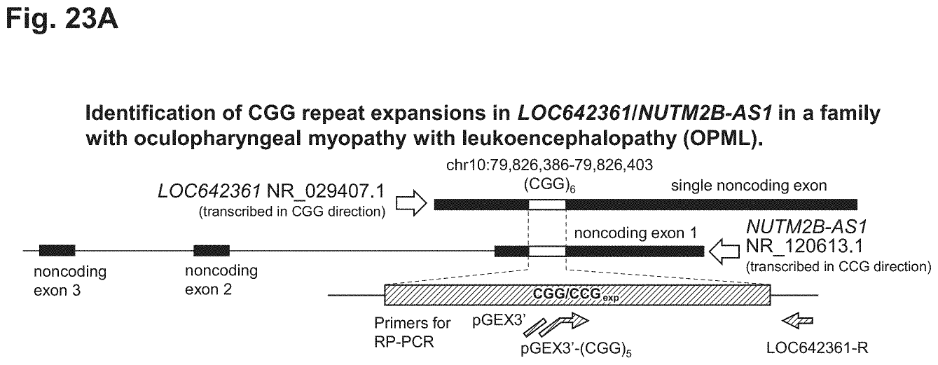

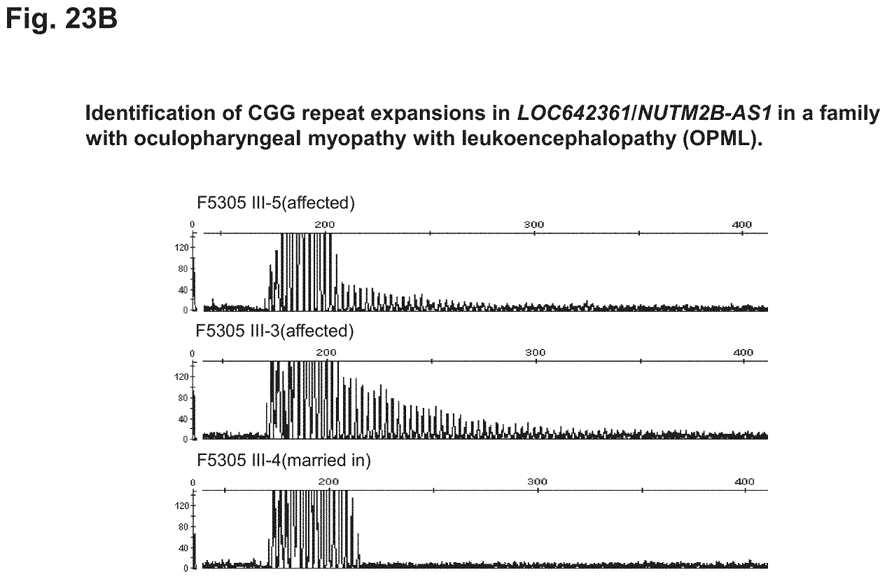

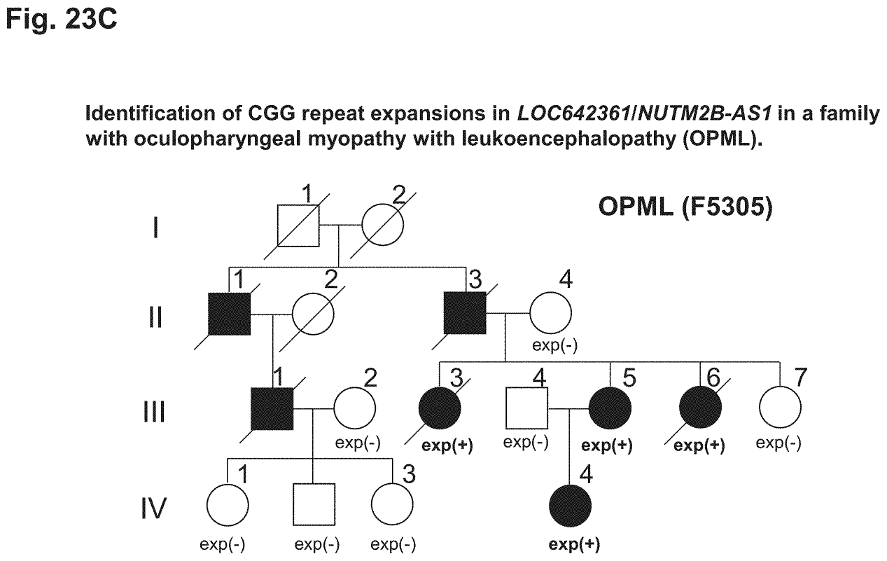

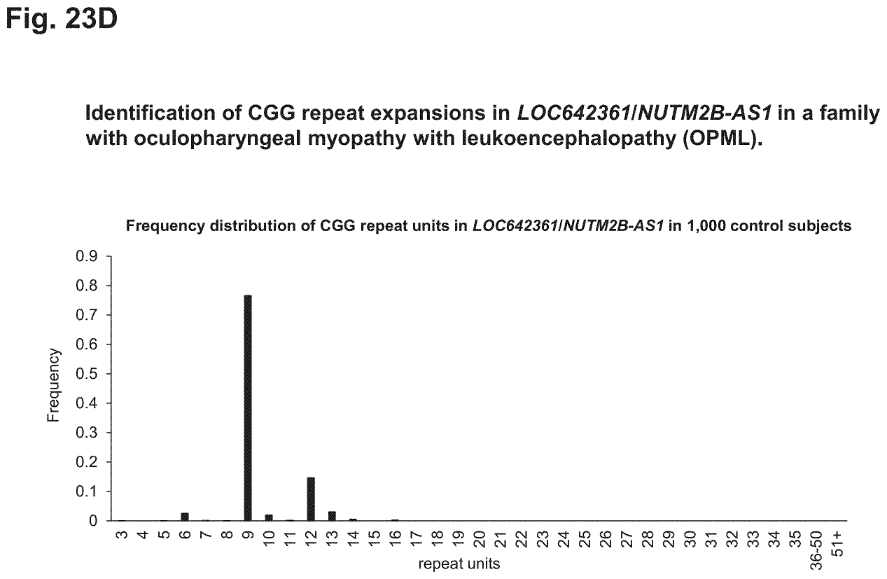

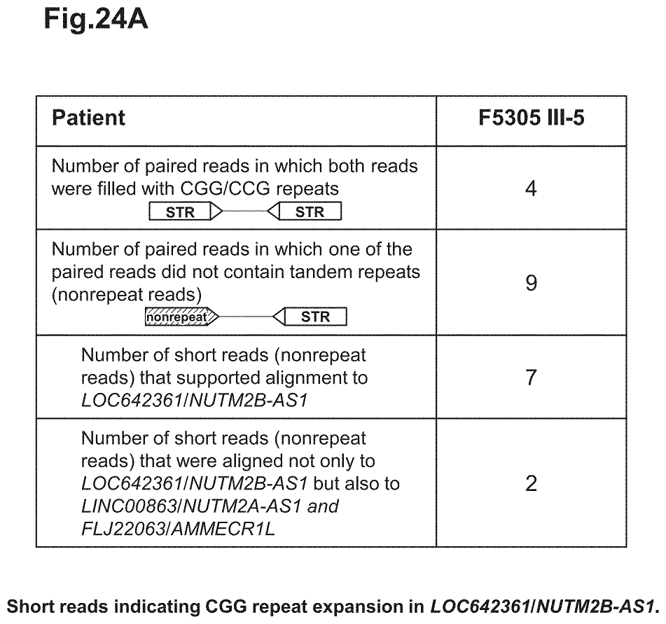

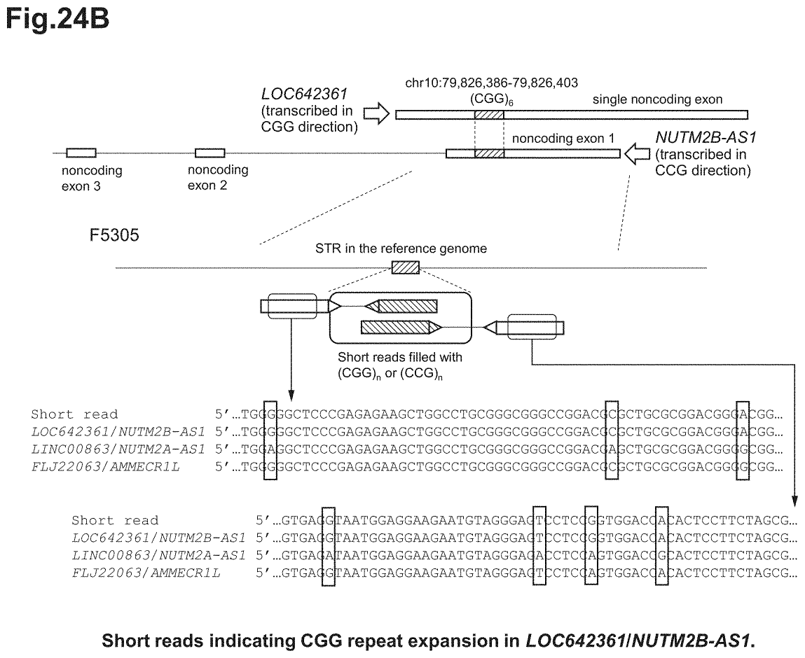

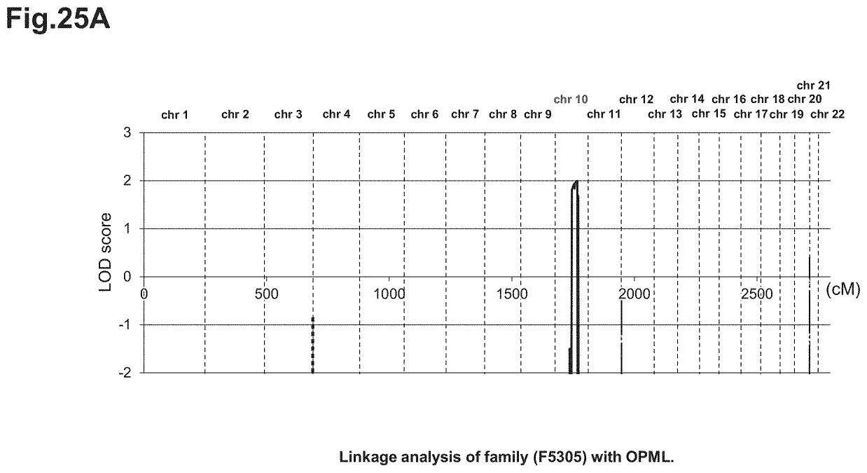

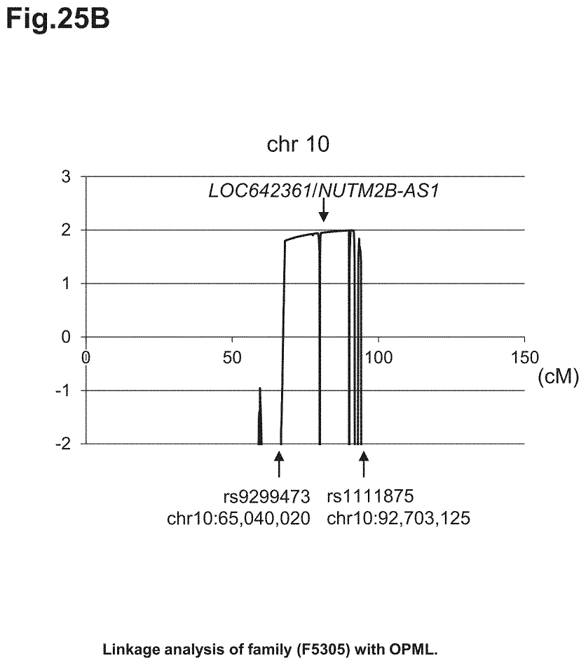

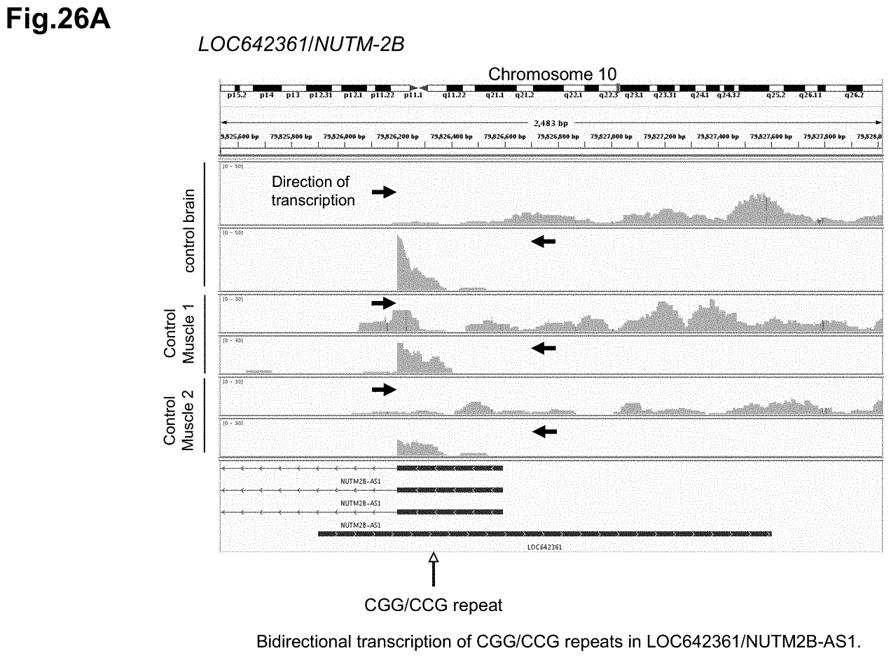

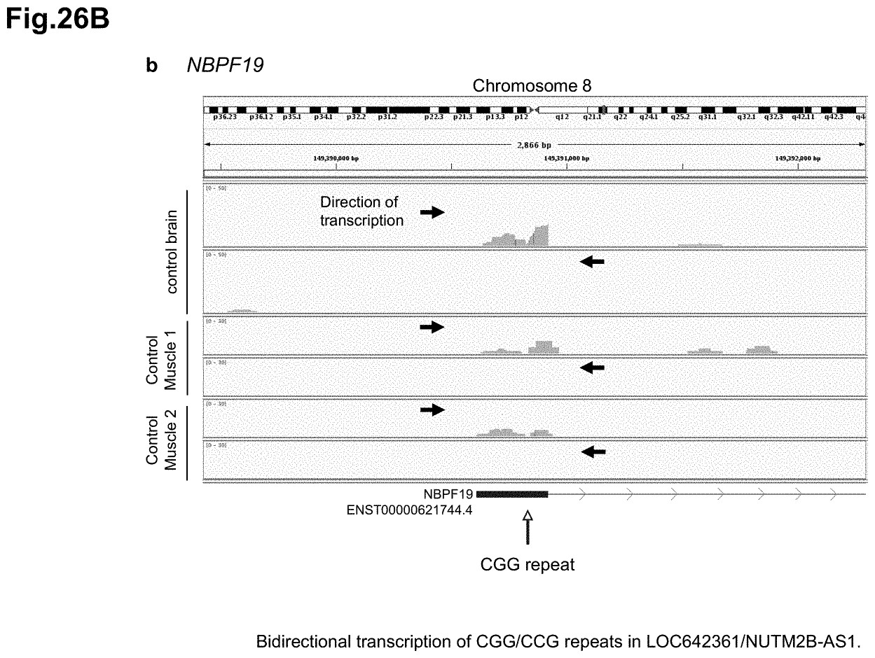

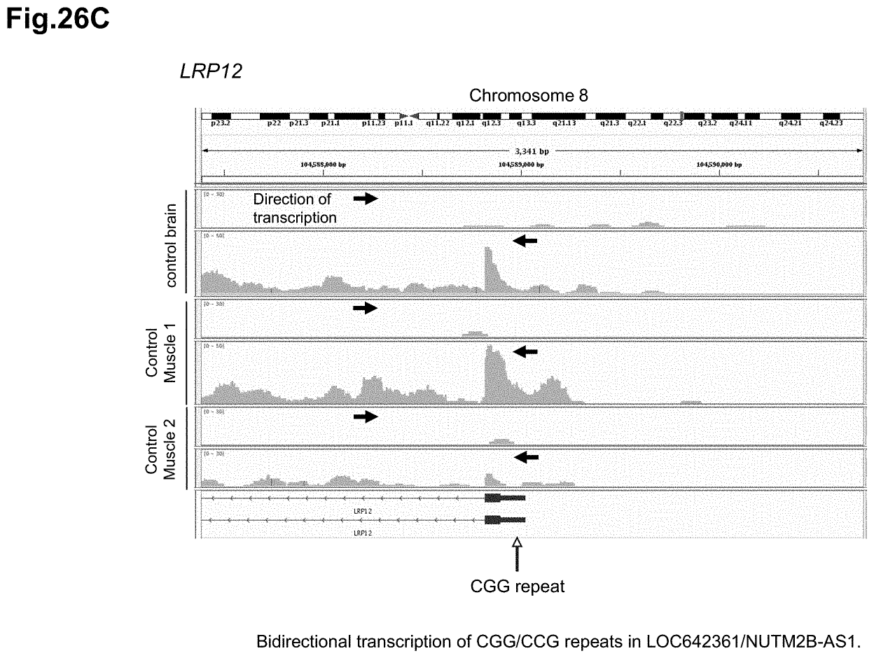

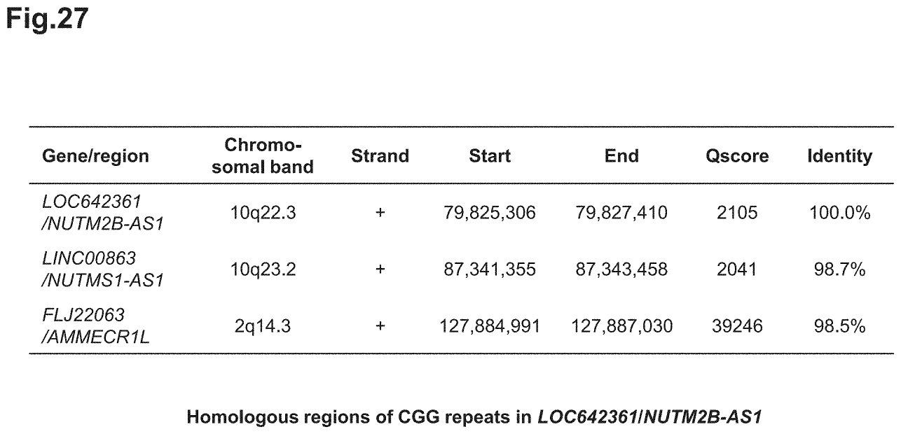

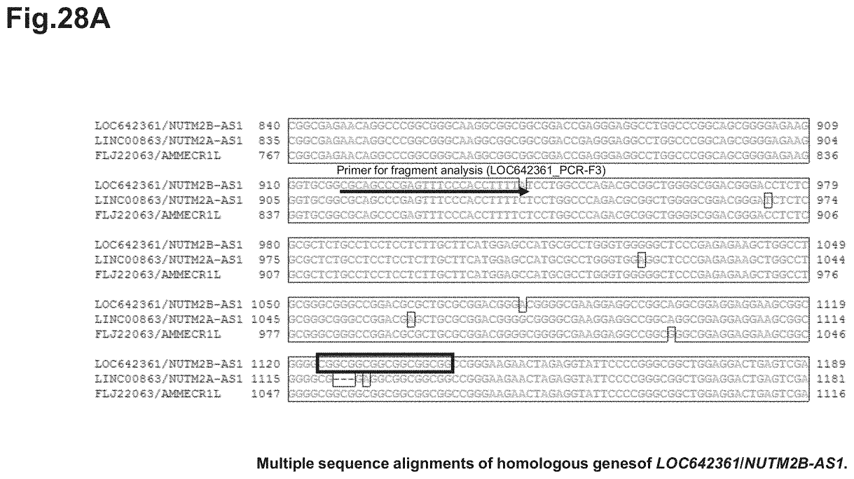

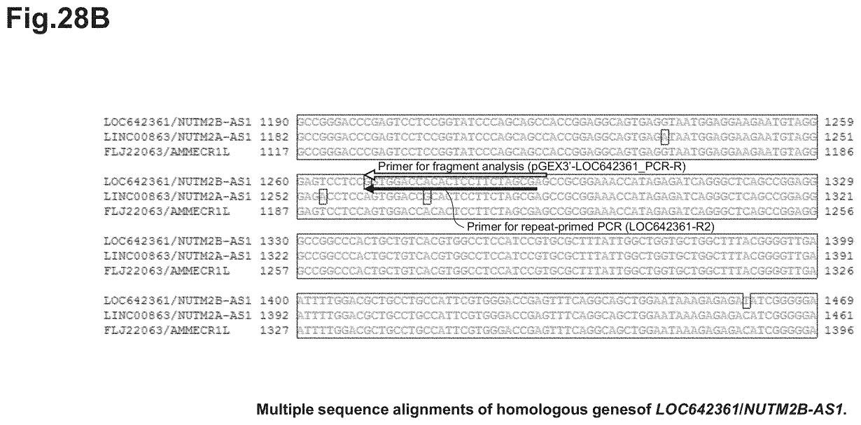

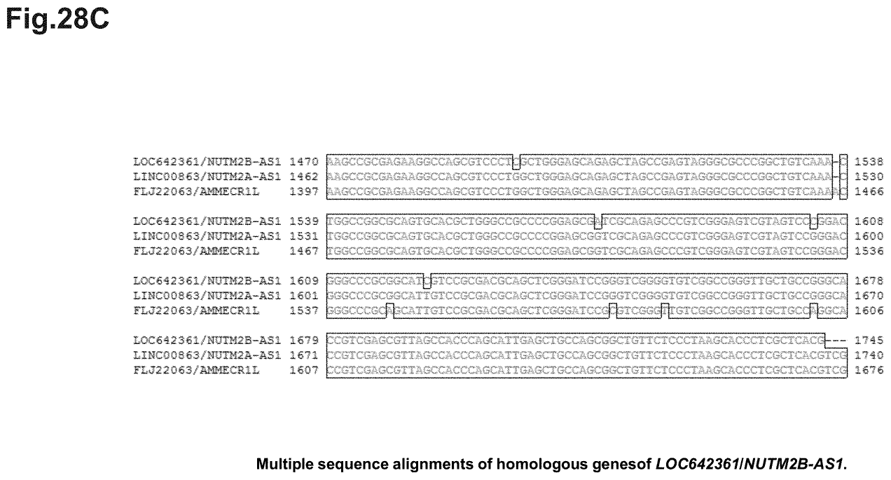

The characteristic MRI findings of NIID include an increased DWI signal intensity in the corticomedullary junction of cerebral white matter. Intriguingly, in a single family (F5305, FIG. 23) presenting with oculopharyngeal myopathy, diffuse limb weakness, and leukoencephalopathy, strikingly similar characteristic DWI findings in the frontal corticomedullary junctions were noted in the index patient (FIG. 1). Patients in the family showed ptosis, restricted eye movements, dysphagia, dysarthria, and diffuse limb muscle weakness with nonspecific myopathic changes in muscle biopsy specimens. MRI was performed in three patients, which revealed T2 hyperintensity signals in the white matter in two patients (III-5 and III-8) and brain atrophy in three patients III-6, and III-8 in F5305). Since this is a new disease entity that has not been previously described, the present inventors designated the disease as oculopharyngeal myopathy with leukoencephalopathy (OPML). Among the patients, two patients (III-3 and III-6) had severe gastrointestinal dysmotility and respiratory failure in addition to ptosis, and ocular, pharyngeal, and limb muscle weakness. Patient III-3 further showed mild ataxia, bladder disturbances, and dilated cardiomyopathy, and patient III-5 showed hand tremor suspected of cerebellar origin. Note that tremor and ataxia are the common clinical characteristics of fragile X tremor/ataxia syndrome (FXTAS) and neuronal intranuclear inclusion disease (MID), and gastrointestinal dysmotility is also occasionally observed in patients with NIID. After CGG repeat expansion mutations in NBPF19 were excluded by repeat-primed PCR analysis, the present inventors similarly directly searched for expanded CGG repeats in the whole-genome sequence data of the patient III-5 using TRhist (FIG. 2) and identified short reads filled with CGG repeats (FIG. 8). The CGG repeat expansion was located in bidirectionally transcribed long noncoding RNAs, LOC642361 (NR_029407.1, transcribed in the CGG direction) and NUTM2B-AS1 (NR_120613.1, transcribed in the CCG direction, FIG. 23 and FIG. 24) on 10q22.3, where parametric linkage analysis showed a single peak with a maximum multipoint LOD score of 1.94 (FIG. 25). Bidirectional transcription was confirmed by stranded RNA-sequence data of a control brain and muscles (FIG. 26). Because the flanking sequences of the CGG repeats in LOC642361/NUTM2B-AS1 have homologous sequences in LINC00863/NUTM2A-AS1 (10q23.2) and FJL22063/AMMECR1L (2q14.3, FIG. 27), the LOC642361/NUTM2B-AS1-specific primers for repeat-primed PCR analysis were designed on (FIG. 28 and FIG. 13). The repeat-primed PCR analysis targeting the CGG repeats confirmed that the four affected individuals in the family had the CGG repeat expansion mutations, whereas the seven unaffected individuals including three married-in healthy individuals did not (FIG. 23). None of the 1,000 controls showed the repeat expansion mutations as determined by repeat-primed PCR analysis. Fragment analysis using an LOC642361/NUTM2B-AS1-specific primer pair (FIG. 17) revealed that the CGG repeats ranged 3-16 in 1,000 controls (FIG. 23).

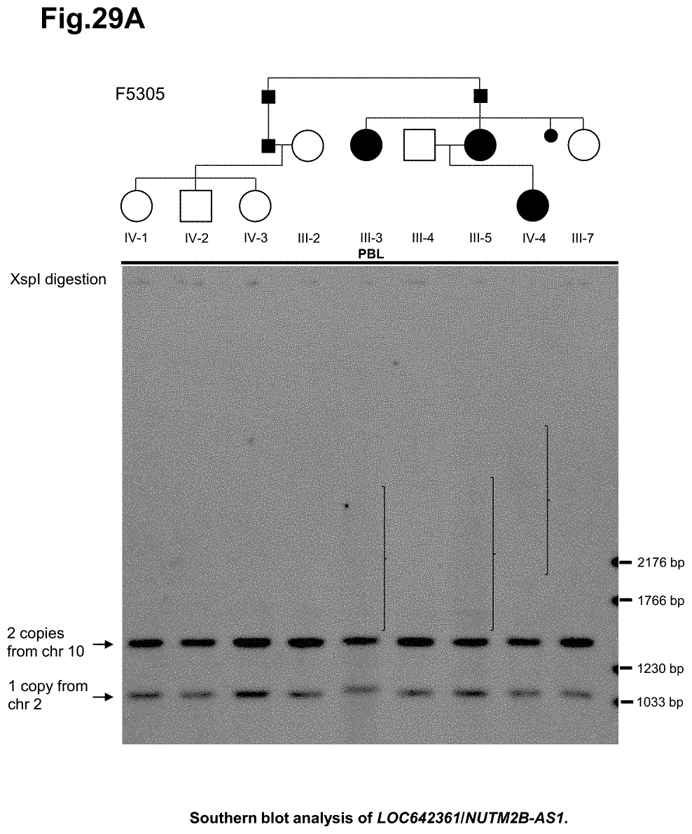

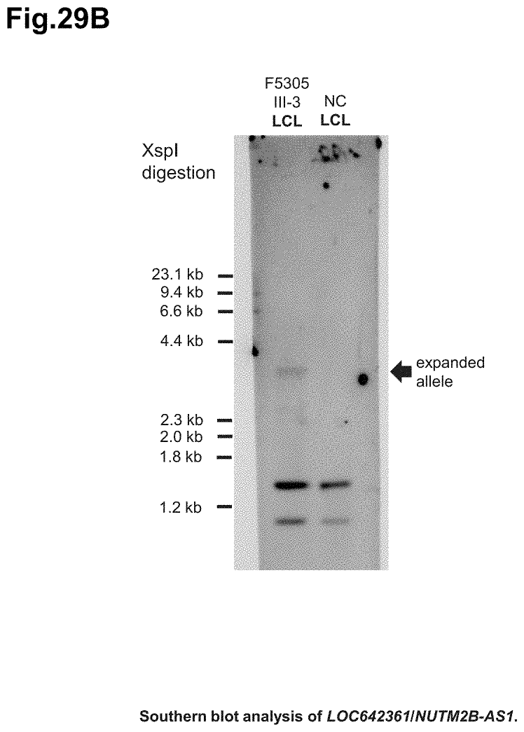

Southern blot analysis of the affected individuals (family F5305) revealed broad smearing patterns (FIG. 15), indicating strong somatic instability of the expanded CGG repeats in LOC642361/NUTM2B-AS1 in genomic DNAs from peripheral blood leukocytes (FIG. 29).

### Example 7: Identification of CGG Repeat Expansions in LRP12 in OPDM

Although cerebral white matter involvement or MCP sign is not observed, another disease, oculopharyngodistal myopathy (OPDM), shared characteristic distributions of muscle involvement including ptosis, external ophthalmoplegia, and dysphagia similar to those of the patients in the family with OPML. Thus, the present inventors further explored a possibility of CGG repeat expansions in families with OPDM. OPDM is an autosomal dominant disease characterized by ptosis, external ophthalmoplegia, and weakness of the masseter, facial, pharyngeal, and distal limb muscles (MIM164310). To date, the causes of OPDM have not been elucidated.

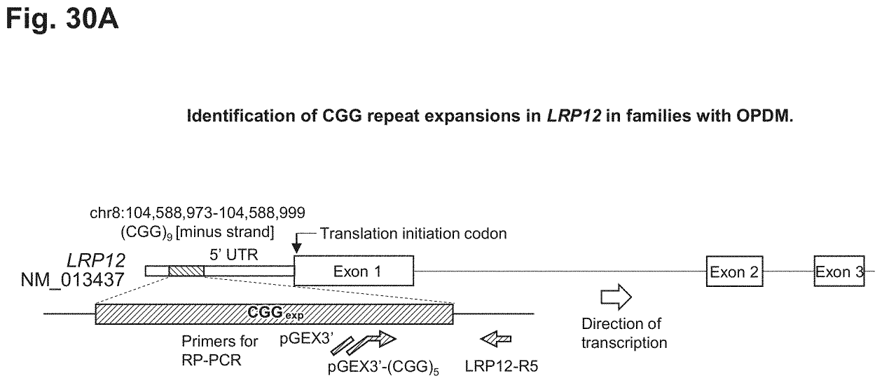

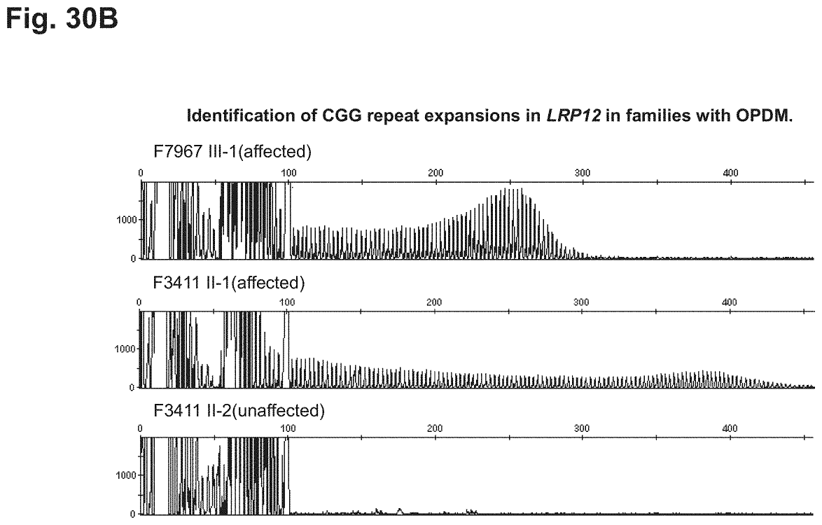

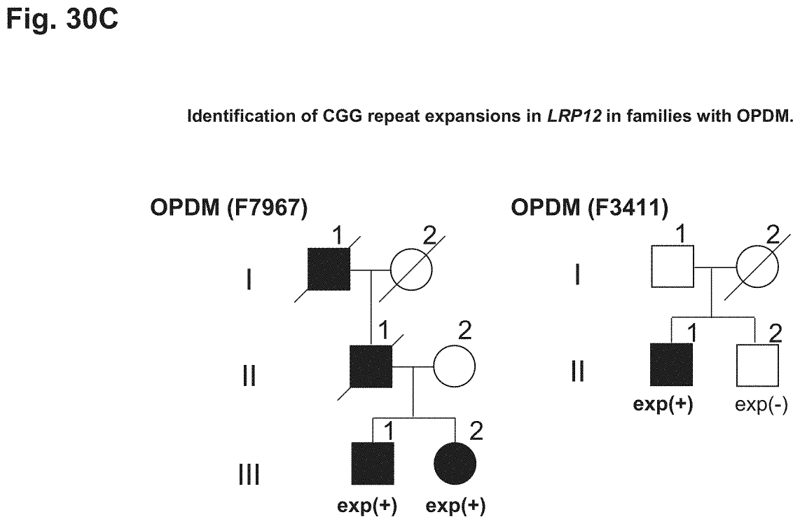

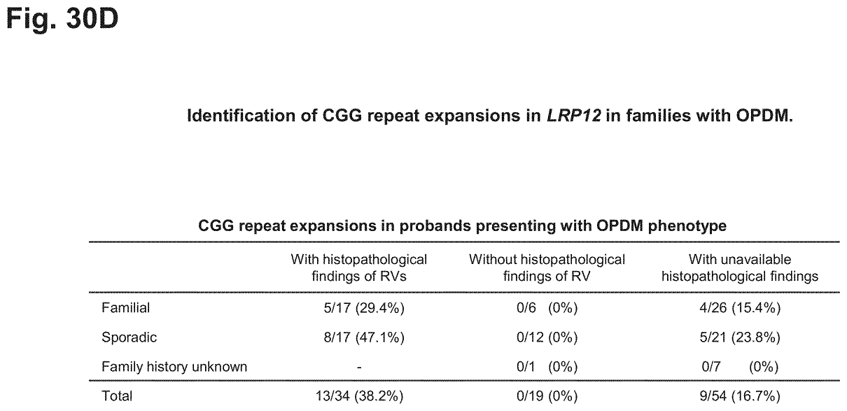

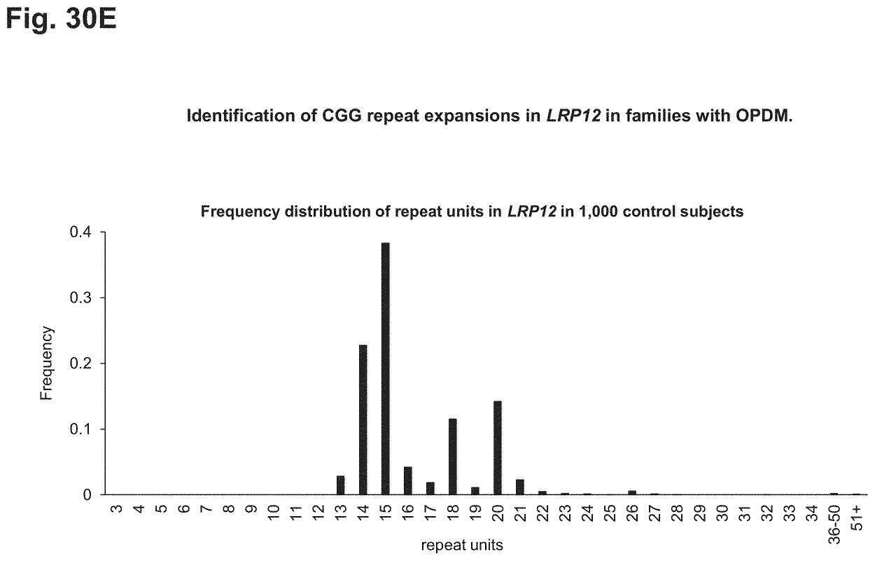

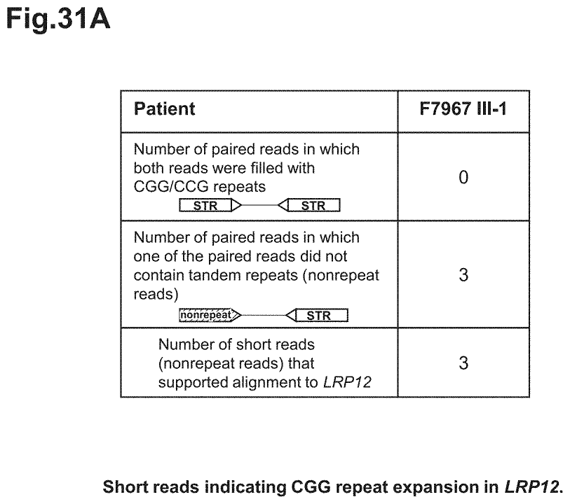

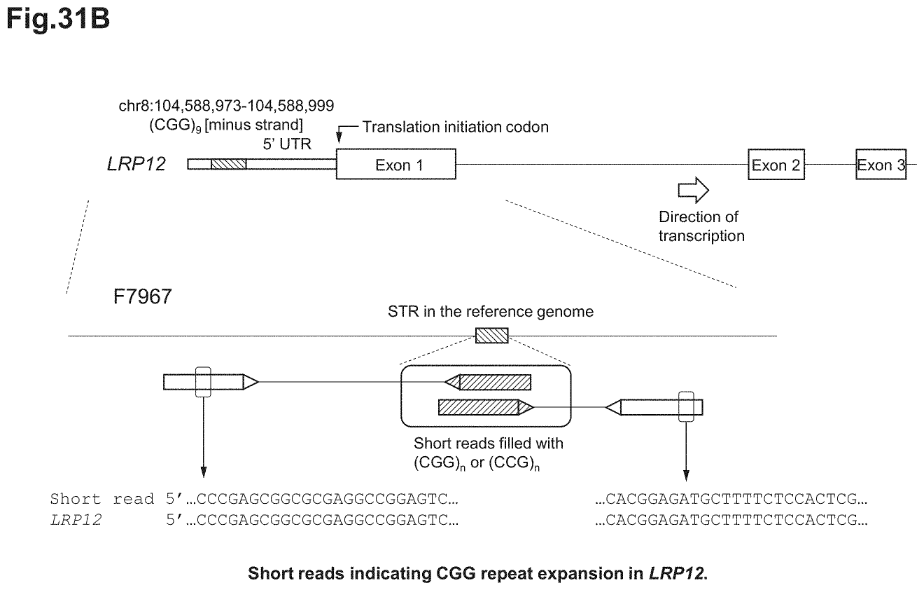

Of the index patients in the 17 families with OPDM and 17 sporadic patients with OPDM in whom biopsied muscle specimens confirmed the presence of myopathic changes with rimmed vacuoles, which is consistent with the diagnosis of OPDM, and GCG repeat expansions in PABPN1, the causative gene for oculopharyngeal muscular dystrophy (OPMD, MIM164300) or CGG repeat expansions in LOC642361/NUTM2B-AS1 were excluded, the present inventors performed whole-genome sequence analysis of patient III-1 of family F7967. Direct search for CGG repeats (FIG. 2) revealed CGG repeat expansions (FIG. 8) located in the 5′ UTR of LRP12, which encodes low density lipoprotein-related protein 12 (NM_013437, FIG. 30 and FIG. 31). Repeat-primed PCR analysis targeting the CGG repeats in LRP12 confirmed the presence of the repeat expansions in patient III-1 in the family F7967 as well as in 12 patients (four with familial OPDM and eight with sporadic OPDM, FIG. 30). The present inventors further screened CGG repeat expansions in the 54 patients exhibiting similar clinical presentations including ptosis, and extraocular and pharyngeal weakness (26 with family history, 21 without family history, and seven with unknown family history) in whom muscle biopsy specimens were unavailable. The repeat-primed PCR analysis targeting CGG repeats in LRP12 revealed nine patients (four familial and five sporadic) with CGG repeat expansions (FIG. 30). In addition, screening for repeat expansions in the other 19 patients with similar muscle involvement but without rimmed vacuoles in biopsied muscle specimens did not reveal CGG repeat expansions in LRP12.

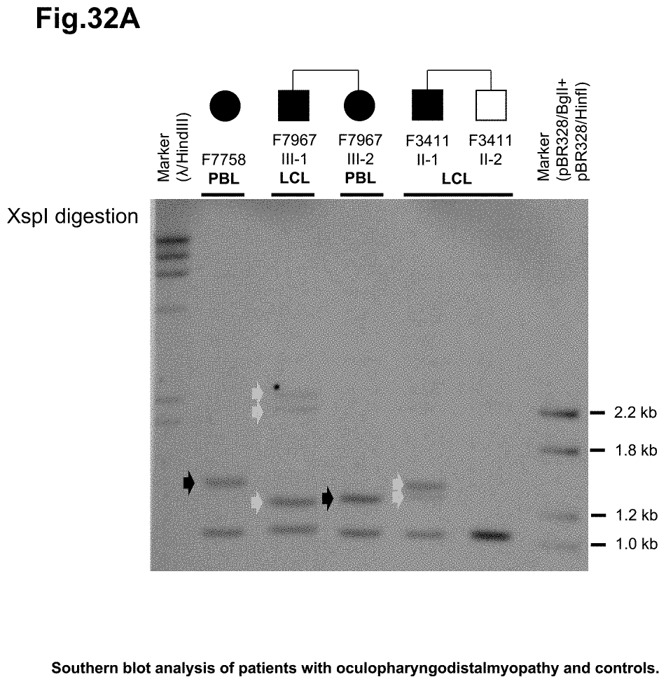

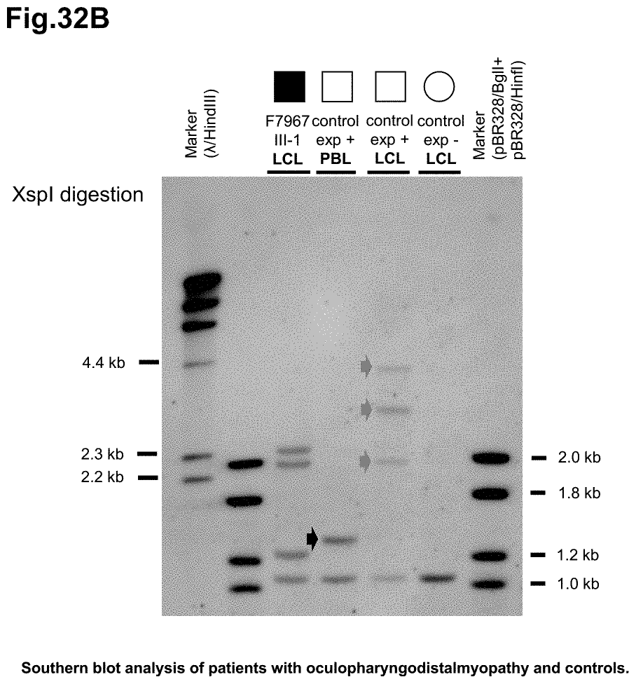

Southern blot analysis (FIG. 15) of four patients with OPDM revealed discrete bands corresponding to the expanded repeats of approximately 280 or 380 bp in genomic DNAs from peripheral blood leukocytes (FIG. 32), while multiple bands corresponding to expanded repeats were observed in genomic DNAs from lymphoblastoid cell lines, indicating somatic instability of the expanded repeats. Affected parent-offspring pairs with OPDM were unavailable.

To determine the distribution of repeat units in controls, the present inventors conducted fragment analysis of the PCR products. As (CGG)₉(CGT)(CGG)(CGT)₂ is registered in hg38, the sizes of the repeats were determined as the total number of repeat units including the repeat sequences flanking (CGG)ₙ. Fragment analysis (FIG. 17) revealed that the number of repeat units in LRP12 ranged 13-45 in 998 controls (FIG. 30), whereas only two of the 1,000 control individuals (0.2%) showed repeat expansions by the repeat-primed PCR analysis, which was further confirmed by Southern blot analysis (FIG. 32).

OPMD, a disease with similar muscle involvement, is caused by short expansions of GCG repeats (affected individuals, 7-14 GCG repeat units; normal individuals, 6 repeat units) encoding a polyalanine stretch in polyadenylate-binding protein 2 (PABP2) encoded by PABPN1. It is intriguing to note that the same repeat motif is expanded in OPMD and OPDM, although the locations of the mutation are different between oculopharyngeal muscular dystrophy (OPMD) (coding region) and OPDM (5′ UTR).

### (Methods)

### (Patients and Controls)

All Japanese index patients were diagnosed as having NIID on the basis of characteristic MRI findings [T2-hyperintensity areas in the middle cerebellar peduncles (MCP sign) and high-intensity signals in DWI in the corticomedullary junction] and/or the presence of ubiquitin-positive intranuclear inclusions in the skin or brain tissues4 (FIG. 6). In multiplex families, those who had cognitive decline and decreased or absent tendon reflexes were considered affected in family members aged >60 years in addition to the index patients with characteristic MRI and/or histopathological findings. Because neuropathy is frequently observed in NIID5, family members with decreased or absent tendon reflexes and decreased motor conduction velocities in nerve conduction study (<49 m/s in the median nerve) were also considered affected. Genomic DNAs of 36 patients with NIID and eight unaffected family members from Japan (FIG. 6), and two patients with NIID from Malaysia were investigated in the study. For confidentiality reason, parts of the pedigree charts were modified not including some individuals with unknown disease status and masking the gender of individuals in the younger generation.

All patients in the Japanese family with OPML showed ptosis, and ocular, pharyngeal, and limb muscle weakness (distal predominant or diffuse weakness). Family members aged over 40 without weakness in ocular or pharyngeal muscles were considered unaffected, because age at onset of the disease is in the range from teenage to 40 years. Genomic DNAs of four affected individuals and seven unaffected individuals in family F5305 were investigated in the study. Other family members were considered to have an unknown disease status.

OPDM was mainly diagnosed clinically. The patients showed characteristic clinical features including ptosis, and ocular, pharyngeal, and distal limb muscle weakness. The present inventors considered that patients in whom muscle biopsy specimens showed myopathic changes with rimmed vacuoles (RVs) were histopathologically supported to have the disease. Genomic DNAs of patients collected in Japan, including 34 with histopathological findings of RVs, 19 without histopathological findings of RVs, and 54 with characteristic clinical features but without histopathological examinations, were investigated in the present inventor's study. In families F7967 and F3411 in which the index patients showed histopathological findings of RVs, genomic DNAs of additional affected and unaffected family members were also investigated in the present inventor's study.

CGG repeat expansion mutations in the 5′ UTR of FMR1 have been excluded in all the probands of NIID (FIG. 14). GCG repeat expansions encoding polyalanine stretches in PABPN1 have been excluded 33 in all the probands with OPML and OPDM.

All the participants gave their informed consent. The present inventor's study was approved by the institutional review boards of the University of Tokyo and the present inventors compiled with all relevant ethical regulations. Genomic DNAs were extracted from peripheral blood leukocytes, lymphoblastoid cell lines, or brains using standard procedures. Control subjects (n=1,000) were collected in Japan.

### (SNV Genotyping)

SNV genotyping using Genome-Wide Human SNP array 6.0 (Affymetrix) was conducted in accordance with the manufacturer's instructions. SNVs were called and extracted using Genotyping Console 3.0.2 (Affymetrix). Only SNVs with p values of >0.05 in the Hardy-Weinberg test in the control samples, call rates of >0.98, and minor allele frequencies of >0.05 were used for further analysis.

### (Genome-Wide Linkage Study)

A genome-wide linkage study of family F5305 (FIG. 30) was performed using the pipeline software SNP-HiTLink and Allegro version 2 with intermarker distances from 80 kb to 120 kb using an autosomal dominant model with complete penetrance. The disease allele frequency was set to 10⁻⁶.

### (Whole-Genome Sequence Analysis and Search for Repeat Sequences)

Whole-genome sequence analysis of patients or controls was performed using HiSeq2500 [Illumina, 150 bp paired end (three patients with NIID, one patient with OPML, one patient with OPDM, and seven controls) or 126 bp paired end (three patients with NIID and a control subject)] in accordance with the manufacturer's instructions using a PCR-free library preparation protocol. Short-read sequences harboring repeat sequences were counted using the TRhist program. Only the reads completely filled with repeat motifs of 3-6 bases without mismatches were counted. Repeat motifs were not included in the tables when less than 10 reads were observed in all the 10 subjects (150 bp) and four subjects (126 bp).

Nonrepeat reads paired with short reads filled with CGG repeats were selected using TRhist. After quality-trimming using sickle (https://github.com/najoshi/sickle), trimmed nonrepeat reads were aligned to hg38 using BLAT. The present inventor annotated transcript/genes using UCSC annotations of RefSeq RNAs (https://genome.ucsc.edu/) or Gencode v29 (https://www.gencodegenes.org/).

(SMRT Sequencing Analysis of a Patient with NIID)

Whole-genome sequence analysis was performed using a Pacific Biosciences Sequel sequencer. Long reads were aligned to the reference genome (hg38) using minimap2 (version 2.10). Multiple sequence alignment analysis of the long reads at the NBPF19 locus including CGG repeat expansions and the five paralogous sequences of the NBPF19, NBPF14, NOTCH2NL, NOTCH2, and AC253572.1 regions obtained from hg38 were performed using ClustalW (version 2.1). The long reads showing CGG repeat expansions in NBPF19 were further polished using Canu (version 1.7) and assembled using racon (version 1.3.1). From the long reads, the present inventors identified CGG repeat expansions in the 5′ UTR of NBPF19 using Tandem Repeat Finder (version 4.0.9).

### (Repeat-Primed PCR Analysis)

Repeat-primed PCR analysis was performed using the primers shown in FIG. 13 and LA taq with GC buffer (TaKaRa). The present inventors used deaza-dGTP in place of dGTP, and slow-down PCR protocol was utilized; initial denaturation at 95° C. for 5 min, followed by 50 cycles of 95° C. for 30 s, 98° C. for 10 s, 62° C. for 30 s, and 72° C. for 2 min. The ramp rate to 95° C. and 72° C. was set to 2.5° C./s and that to 62° C. was set to 1.5° C./s. Fragment analysis was performed using an ABI PRISM 3130x1 or 3730 sequencer (Life Technologies) and data were analyzed using GeneMapper software (version 4.1, Life Technologies).

### (Southern Blot Analysis)

Southern blot analysis was performed to detect CGG repeat expansions in NBPF19, LOC642361/NUTM2B-AS1, and LRP12. The probes were designed to target the flanking regions of the CGG repeats in the 5′ UTR of NBPF19, the noncoding exon in LOC642361/NUTM2B-AS1, and the 5′ UTR of LRP12. Genomic fragments were subcloned into plasmids (pTA2, Toyobo) using primers shown in FIG. 15, and probes were prepared by digoxigenin (DIG) labeling PCR using DIG-dUTP and dTTP at a ratio of 0.7 to 1.3. To increase signal intensity, several probes (Probes 1-5 or Probes 7 and 8) were mixed for hybridization for NBPF19 or LRP12, respectively. The primer pairs used for DIG-labeling are shown in FIG. 15.

Ten μg of Genomic DNAs Extracted from Peripheral Blood Leukocytes or Lymphoblastoid Cell Lines was Digested with SacI and/or NheI (NBPF19) or XspI (LOC642361/NUTM2B-AS1 and LRP12) and electrophoresed in 0.8%-1.2% agarose gels followed by capillary blotting onto positively charged nylon membranes (Sigma-Aldrich) and cross-linking by exposure to ultraviolet light. After prehybridization, the probes were hybridized overnight at 42° C. (LOC642361/NUTM2B-AS1 and LRP12) or 48° C. (NBPF19) in DIG Easy Hyb (Sigma-Aldrich). The membrane was finally washed with 0.1×-0.5× saline sodium citrate (SSC) and 0.1% sodium dodecyl sulfate (SDS) in 68° C. twice for 15 min each. The detection process was performed using Fab fragments of an anti-DIG antibody conjugated to alkaline phosphatase (Sigma-Aldrich), CDP-star (Sigma-Aldrich), and LAS3000 mini (Fujifilm)

### (Analysis of Repeat Sizes in Controls)

The present inventors conducted fragment analysis to determine distribution of sizes of CGG repeats in NBPF19, LOC642361/NUTM2B-AS1, and LRP12 in 1,000 controls (FIG. 17). In the analysis of NBPF19 and LOC642361/NUTM2B-AS1, the present inventors used NBPF19- and LOC642361/NUTM2B-AS1-specific primers to avoid non-specific amplification of genes due to highly homologous sequences (FIG. 17).

To determine the repeat configurations of CGG repeats in NBPF19, the present inventors conducted circular consensus sequencing (CCS) analysis using a PacBio Sequel sequencer (Pacific Biosciences) for pooled barcoded PCR products containing the CGG repeats in NBPF19 (FIG. 18) that were prepared from 194 control subjects. “By strand” CCS reads were generated using SMRT Link (v.6.0.0.47841) Minimum number of passes were set to be 20 to obtain accurate CCS reads. After discarding 12 subjects with less than 50 CCS reads, the present inventors were able to determine number of CGG repeat units, repeat configurations, and flanking sequences in the 182 control subjects. In this analysis, copy number variations involving this locus were not taken into consideration.

### (Methylation Analysis Using SMRT Sequencing Reads)

To investigate the CpG methylation status of expanded CGG repeats in the 5′ UTR of NBPF19, the present inventors utilized kinetic metric called inter-pulse duration (IPD) from SMRT sequencing reads. The present inventors first created a reference IPD set for the hypomethylated CGGs and hypermethylated CGGs using whole-genome bisulfite sequencing data and SMRT sequencing data obtained from the same control individual. CGG repeats in the hg38 reference sequence were identified by aligning synthetic (CGG)ₙ sequence (n=7; 21 bp) to the reference by Bowtie 2 (version 2.1.0) allowing no mismatches. After removing regions without enough PacBio reads for calculating IPD statistics according to SMRT Pipe (version 0.51.0) provided by Pacific Biosciences, the present inventors obtained 401 CGG repeat sites. Then, the present inventors associated each CpG site with methylation status obtained by whole genome bisulfite sequencing data. The present inventors had, however, a smaller number of bisulfite-treated short reads available on CGG repeats than on other unique regions presumably due to ambiguous short read alignment to CGG repeats or high GC content. Since methylation statuses of neighboring CpG sites are likely to be correlated, the present inventors assumed that CpG sites in a single CGG repeat had an identical methylation status; namely, if <30% (>70%, respectively) of bisulfite calls on CpG sites within the repeat support methylation, then the entire region was defined to be hypomethylated (hypermethylated) as a whole. The analysis revealed 303 hypomethylated CGG repeat regions with 1,220 CpGs and 14 hypermethylated regions with 59 CpGs. The present inventors observed a significant difference in IPD statistics at cytosine of CGG between the hypermethylated and hypomethylated CpG sites (p=3.3*10⁻¹⁶) using Mann-Whitney U test (one-sided), demonstrating that IPD is informative in inferring CpG methylation statues of CGG repeat (FIG. 21).

The present inventors next examined whether the CGG repeats in the 5′ UTR of NBPF19 in a patient were similar to hypomethylated CGG repeat or hypermethylated CGG repeat in terms of IPD statistics of CpG sites, and the present inventors examined the null hypothesis of independence of IPD statistics using Mann-Whitney U test.

(RNA-Seq Analysis in Brains of Patients with NIID and Control Subjects)

To determine the expression levels of NBPF19 in patients with NIID, three autopsied brains of patients with NIID as well as eight control brains (occipital lobe) were subjected to unstranded RNA-seq. Short reads were aligned to hg38 using STAR (version 2.5.3a) and the numbers of reads aligned to NBPF19-specific sequences among the five homologous sequences were visually investigated. Statistical analysis was performed using Wilcoxon's rank sum test (two-sided).

To examine transcriptional directions, data on stranded RNA-seq of normal subjects (brain, n=1; muscle, n=2) were aligned to hg38 using STAR (version 2.5.3a). After reads with mapping quality of less than five were discarded using SAMtools (version 1.6), aligned reads and coverages were visualized using the Integrative Genomics Viewer (version 2.4.4).

### (Haplotype Analysis)

Disease-relevant haplotypes in three families with OPDM (F3411, F7758, and F7967) were reconstructed using SNP genotypes. In addition, employing linked-read analysis (10X GemCode Technology), the haplotypes of the patient II-1 in family F3411, the index patient in family F7758, and the patient III-1 in family F7967 were determined using longranger (version 2.1.6) and loupe (version 2.1.1). The present inventors used the reference genome hg19 in this analysis.

(Summary of Clinical Presentation of the Index Patient (III 3) in Family F5305 with Oculopharyngeal Myopathy with Leukoencephalopathy (OPML)

The pedigree chart of this family (F5305) is shown in FIG. 23. There are seven affected individuals consistent with autosomal dominant inheritance.

The index patient (III 3, FIG. 23 noticed nasal voice a t the age of 15. The progression of her symptom was as follows: at 27 years old (y/o), she began noticing easy fatigability of her extremities; at 30 y/o, ptosis; and at 32 y/o, mild dysphagia. She underwent repeated blepharoplasties at ages 34, 45, and 56. She was examined at another hospital a t 35 y/o, where ptosis, dysarthria, dysphagia, and weakness of facial and neck muscles were observed, however, the limb muscles were minimally involved. Needle electromyography revealed motor units with short duration and low voltage, which were considered as myogenic changes. Muscle biopsy revealed no abnormal findings. Motor nerve conduction studies were normal.

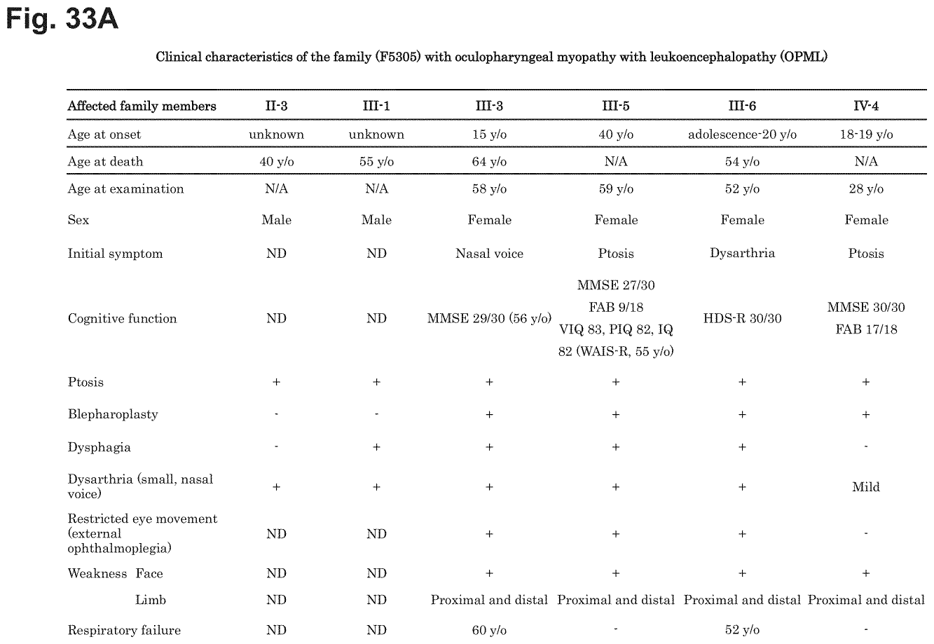

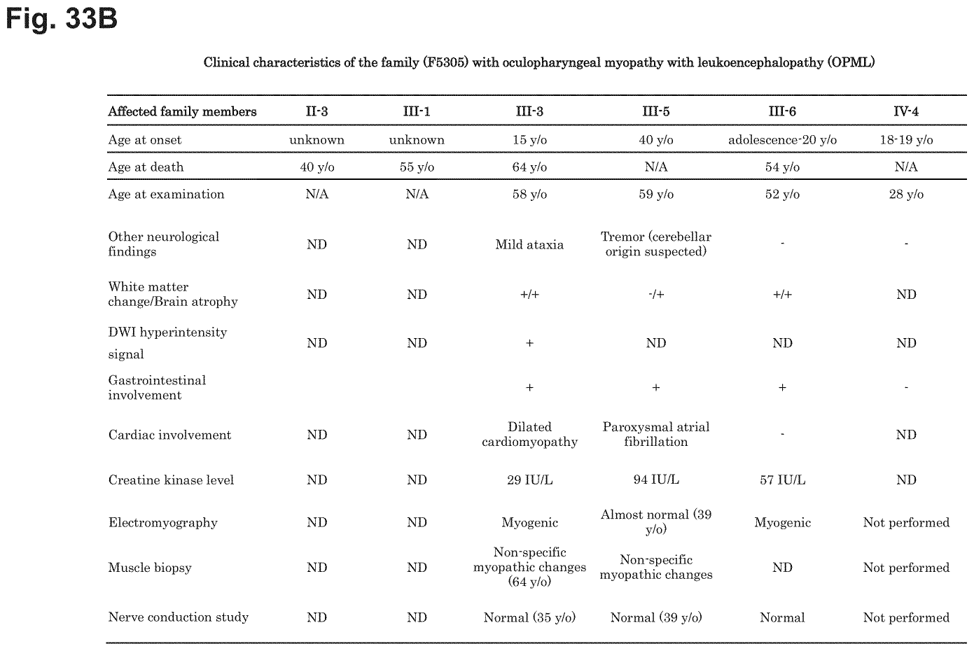

Her symptoms gradually progressed. Detailed examination s at 58 y/o at the Department of Neurology, The University of Tokyo Hospital revealed ptosis, near lycomplete external ophthalmoplegia, dysarthria with nasal voice, and dysphagia. She also had facial, neck, and diffuse limb muscle weakness accompanied with diffuse muscular atrophy and generalized are flexia. She had dysuria requiring abdominal pressure to assist urination. Although tube feeding was tried because of dysphagia and repeated aspiration pneumonia, tube enteral feeding was not adequate due to severe gastrointestinal dysmotility. Weakness of respiratory muscles led to hypercapnia. On laboratory examination, serum creatine kinase levels were below the lower limit (29IU/L) L), while serum lactate and pyruvate levels were normal. Echocardiography revealed diffuse hypokinesis of the left ventricle (ejection fraction of 44%). Magnetic resonance imaging of the head revealed T2 hyperintensity signals in the white matter accompanied with hyperintensity signals on diffusion weighted images in the corticomedullary junction (FIG. 1). Clinical presentation of other family members are summarized in FIG. 33.

Although autosomal dominant mitochondrial diseases exhibiting chronic progressive external ophthalmoplegia were initially considered FIG. 23) from the pedigree chart, no rearrangement s or deletions of mitochondrial DNA were identified by Southern blot hybridization analysis of genomic DNA extracted from the abdominal muscle specimen. Causative mutations in the nuclear genes responsible for autosomal dominant mitochondrial diseases POLG, SLC25A4, C10ORF2, POLG2, RRM2B, DNA2, OPA1, and AFG3L2 were not identified by whole genome sequence analysis. Oculopharyngeal muscular dystrophy was excluded by the analysis of the CGG repeat in PABPN1. Although oculopharyngodistal myopathy (OPDM) was another differential diagnosis, patients with OPDM usually showed muscular weakness with predominance in distal limbs and rimmed vacuoles in muscle biopsy specimens 1, while the patients in this family did not show such findings. Involvement of the gastrointestinal tract 2 or the heart 3 was only infrequently observed in patients with OPDM. Taken together with myopathy of the oculopharyngeal type, diffuse muscular weakness, characteristic brain MRI findings (leukoencephalopathy), and the gastrointestinal involvement, the present inventors considered the characteristic clinical presentation in this family constitute a novel clinical entity and designate the disease as OPML.

### SEQUENCE LISTING

★

1. A method for determining a neuromuscular disease accompanied with a repeat expansion of CGG in a nucleic acid in a subject comprising detecting a repeat expansion of CGG or a complementary sequence thereof in a nucleic acid sample from the subject.

2. The method of claim 1, wherein the neuromuscular disease is selected from the group consisting of neuronal intranuclear inclusion disease, oculopharyngodistal myopathy, and oculopharyngeal myopathy with leukoencephalopathy.

3. The method of claim 1, wherein the nucleic acid sample is a chromosome DNA.

4. The method of claim 1, wherein the repeat expansion of CGG is in an intron of a gene from the subject.

5. The method of claim 4,
wherein the neuromuscular disease is neuronal intranuclear inclusion disease, and
wherein the repeat expansion of CGG is in 5′ untranslated region of NBPF19 gene.

6. The method of claim 5, wherein the repeat expansion is greater than 70 repeats.

7. The method of claim 4,
wherein the neuromuscular disease is oculopharyngodistal myopathy, and
wherein the repeat expansion of CGG is in 5′ untranslated region of LRP12 gene.

8. The method of claim 7, wherein the repeat expansion is greater than 70 repeats.

9. The method of claim 4,
wherein the neuromuscular disease is oculopharyngeal myopathy with leukoencephalopathy, and
wherein the repeat expansion of CGG is in 5′ untranslated region of LOC642361 gene and/or NUTM2B-A S1 gene.

10. The method of claim 9, wherein the repeat expansion is greater than 70 repeats.

11. A kit for determining a neuromuscular disease accompanied with a repeat expansion of CGG in a nucleic acid in a subject comprising a nucleic acid reagent configured to detect a repeat expansion of CGG or a complementary sequence thereof in a nucleic acid sample from the subject.

12. The kit of claim 11, wherein the neuromuscular disease is selected from the group consisting of neuronal intranuclear inclusion disease, oculopharyngodistal myopathy, and oculopharyngeal myopathy with leukoencephalopathy.

13. The kit of claim 11, wherein the nucleic acid sample is a chromosome DNA.

14. The kit of claim 11, wherein the nucleic acid reagent comprises a PCR primer configured to detect the repeat expansion of CGG or the complementary sequence thereof.

15. The kit of claim 14, wherein the PCR primer comprises a complementary sequence of CGG or a complementary sequence thereof.

16. The kit of claim 11, wherein the nucleic acid reagent comprises a probe configured to target a sequence flanking the repeat expansion of CGG or a complementary sequence thereof.

17. The kit of claim 11, wherein the repeat expansion of CGG is in an intron of a gene from the subject.

18. The kit of claim 17,
wherein the neuromuscular disease is neuronal intranuclear inclusion disease, and
wherein the repeat expansion of CGG is in 5′ untranslated region of NBPF19 gene.

19. The kit of claim 18 wherein the repeat expansion is greater than 70 repeats.

20. The kit of claim 17,
wherein the neuromuscular disease is oculopharyngodistal myopathy, and
wherein the repeat expansion of CGG is in 5′ untranslated region of LRP12 gene.

21. The kit of claim 20, wherein the repeat expansion is greater than 70 repeats.

22. The kit of claim 17,
wherein the neuromuscular disease is oculopharyngeal myopathy with leukoencephalopathy, and
wherein the repeat expansion of CGG is in 5′ untranslated region of LOC642361 gene and/or NUTM2B-A S1 gene.

23. The kit of claim 22, wherein the repeat expansion is greater than 70 repeats.
